(19) United States
(12) Patent Application Publication
(10) Pub. No.: US 2024/0207264 A1
(43) Pub. Date: Jun. 27, 2024

(54) PHARMACEUTICAL COMPOSITIONS COMPRISING INHIBITORS OF THE ANDROGEN RECEPTOR AND USES THEREOF

(71) Applicant: ESSA PHARMA INC., Vancouver (CA)

(72) Inventors: Han-Jie ZHOU, Foster City, CA (US); Peter VIRSIK, Portola Valley, CA (US)

(21) Appl. No.: 18/555,599

(22) PCT Filed: Apr. 15, 2022
(86) PCT No.: PCT/US2022/025016

§ 371 Date: Oct. 16, 2023

Related U.S. Application Data
(60) Provisional application No. 63/176,044, filed on Apr. 16, 2021.

(51) Int. Cl. A61K 31/505; A61K 9/20; A61P 35/00; C07D 239/42

(52) U.S. Cl. CPC A61K 31/505; A61K 9/2013; A61K 9/2018; A61K 9/2027; A61K 9/2054; A61P 35/00; C07D 239/42

(57) ABSTRACT

The present disclosure generally relates to pharmaceutical compositions comprising N-(4-((4-(2-(3-chloro-4-(2-chloroethoxy)-5-cyanophenyl) propan-2-yl)phenoxy)methyl)pyrimidin-2-yl)methanesulfonamideN-(4-((4-(2-(3-chloro-4-(2-chloroethoxy)-5-cyanophenyl) propan-2-yl)phenoxy)methyl)pyrimidin-2-yl)methanesulfonamide or a pharmaceutically acceptable salt, solvate, stereoisomer, or prodrug thereof. In particular, the present disclosure relates to solid dispersion pharmaceutical compositions useful for treatment of various cancers, for example breast cancer and prostate cancer.

### CROSS-REFERENCE TO RELATED APPLICATIONS

This application claims the benefit of and priority to U.S. Provisional Application No. 63/176,044 filed Apr. 16, 2021, which is hereby incorporated by reference in its entirety.

### FIELD OF THE INVENTION

The present disclosure generally relates to pharmaceutical compositions comprising an androgen receptor (AR) N-terminal domain inhibitor (NTD) Compound A. In particular, the present disclosure relates to pharmaceutical compositions in the form of solid dispersion composition useful for treatment of various cancers, such as prostate cancer.

### BACKGROUND OF THE INVENTION

Androgens mediate their effects through the androgen receptor (AR). Androgens play a role in a wide range of developmental and physiological responses and are involved in male sexual differentiation, maintenance of spermatogenesis, and male gonadotropin regulation (R.

K. Ross, G. A. Coetzee, C. L. Pearce, J. K. Reichardt, P. Bretsky, L. N. Kolonel, B. E, Henderson, E. Lander, D. Altshuler & G. Daley, Eur Urol 35, 355-361 (1999); A. A. Thomson, Reproduction 121, 187-195 (2001); N. Tanji, K. Aoki & M. Yokovama Arch Androl 47, 1-7 (2001)). Several lines of evidence show that androgens are associated with the development of prostate carcinogenesis. Firstly, androgens induce prostatic carcinogenesis in rodent models (R. L. Noble, Cancer Res 37, 1929-1933 (1977); R. L. Noble, Oncology 34, 138-141 (1977)) and men receiving androgens in the form of anabolic steroids have a higher incidence of prostate cancer (J. T, Roberts & D. M. Essenhigh, Lancet 2, 742 (1986); J. A. Jackson, J. Waxman & A. M. Spiekerman, Arch Intern Med 149, 2365-2366 (1989); P. D. Guinan, W. Sadoughi, H. Alsheik, R. J. Ablin, D. Alrenga & I. M. Bush, Am J Surg 131, 599-600 (1976)). Secondly, prostate cancer does not develop if humans or dogs are castrated before puberty (J. D. Wilson & C. Roehrborn, J Clin Endocrinol Metab 84, 4324-4331 (1999); G. Wilding, Cancer Surv 14, 113-130 (1992)). Castration of adult males causes involution of the prostate and apoptosis of prostatic epithelium while eliciting no effect on other male external genitalia (E. M. Bruckheimer & N. Kyprianou, Cell Tissue Res 301, 153-162 (2000); J. T. Isaacs, Prostate 5, 545-557 (1984)). This dependency on androgens provides the underlying rationale for treating prostate cancer with chemical or surgical castration also known as androgen ablation therapy (ABT) or androgen deprivation therapy (ADT).

Androgen receptor (AR) is a transcription factor that plays dual roles in breast cancer cells: promoting or inhibiting proliferation depending on expression and activity of estrogen receptor-alpha. Currently there is no FDA-approved targeted therapy for triple-negative breast cancer (TNBC). AR plays a role in the proliferation of breast cancer cells by either promoting proliferation or inhibiting proliferation depending on the expression of estrogen receptor (ER) and human epidermal growth factor receptor 2 (HER2). AR expression is detected in up to 90% of all breast cancers and in up to approximately 35% of TNBC. AR-Vs have been detected in primary breast cancer specimens and in breast cancer cell lines. AR-V7 expression was detected in circulating-tumor cells of patients with metastatic breast cancer and was associated with bone metastases. Targeting AR is a potential therapeutic strategy for AR-positive TN BC.

Androgens also play a role in female diseases such as polycystic ovary syndrome as well as cancers, One example is ovarian cancer where elevated levels of androgens are associated with an increased risk of developing ovarian cancer (K. J. Helzlsouer, A. J. Alberg, G. B. Gordon, C. Longcope, T. L. Bush, S. C. Hoffman & G. W. Comstock, JAMA 274, 1926-1930 (1995); R. J. Edmondson, J. M. Monaghan & B. R. Davies Br J. Cancer 86, 879-885 (2002)). The AR has been detected in a majority of ovarian cancers (H. A. Risch, J Natl Cancer Inst 90, 1774-1786 (1998); B. R. Rao & B. J. Slotman, Endocr Rev 12, 14-26 (1991); G. M. Clinton & W. Hua, Crit Rev Oncol Hematol 25, 1-9 (1997)), whereas estrogen receptor-alpha (ERa) and the progesterone receptor are detected in less than 50% of ovarian tumors.

The only effective treatment available for advanced prostate cancer is the withdrawal of androgens which are essential for the survival of prostate luminal cells. Androgen ablation therapy causes a temporary reduction in tumor burden concomitant with a decrease in serum prostate-specific antigen (PSA). Unfortunately, prostate cancer can eventually grow again in the absence of testicular androgens (castration-resistant disease) (Huber et al 1987 Scand J Urol Nephrol. 104, 33-39). Castration-resistant prostate cancer that is still driven by AR is biochemically characterized before the onset of symptoms by a rising titre of serum PSA (Miller et al 1992 J. Urol. 147, 956-961). Once the disease becomes castration-resistant most patients succumb to their disease within two years.

The AR has distinct functional domains that include the carboxy-terminal ligand-binding domain (LBD), a DNA-binding domain (DBD) comprising two zinc finger motifs, and an N-terminus domain (NTD) that contains two transcriptional activation units (tau1 and tau5) within activation function-1 (AF-1). Binding of androgen (ligand) to the LBD of the AR results in its activation such that the receptor can effectively bind to its specific DNA consensus site, termed the androgen response element (ARE), on the promoter and enhancer regions of “normally” androgen regulated genes, such as PSA, to initiate transcription. The AR can be activated in the absence of androgen by stimulation of the cAMP-dependent protein kinase (PKA) pathway, with interleukin-6 (IL-6) and by various growth factors (Culig et al 1994 Cancer Res. 54, 5474-5478; Nazareth et al 1996 J. Biol. Chem. 271, 19900-19907; Sadar 1999 J. Biol. Chem. 274, 7777-7783; Ueda et al 2002 A J. Biol. Chem. 277, 7076-7085; and Ueda et al 2002 B J. Biol. Chem. 277, 38087-38094). The mechanism of ligand-independent transformation of the AR has been shown to involve: 1) increased nuclear AR protein suggesting nuclear translocation; 2) increased AR/ARE complex formation; and 3) the AR-NTD (Sadar 1999 J. Biol. Chem. 274, 7777-7783; Ueda et al 2002 A J. Biol. Chem. 277, 7076-7085; and Ueda et al 2002 B J. Biol. Chem. 277, 38087-38094). The AR can be activated in the absence of testicular androgens by alternative signal transduction pathways in castration-resistant disease, which is consistent with the finding that nuclear AR protein is present in secondary prostate cancer tumors (Kim et al 2002 Am. J. Pathol. 160, 219-226; and van der Kwast et al 1991 Inter. J. Cancer 48, 189-193).

Clinically available inhibitors of the AR include nonsteroidal antiandrogens such as bicalutamide (Casodex™), nilutamide (Anandron®, Nilandron®), flutamide (Eulexin®), enzalutamide (Xtandi®), apalutamide (Erleada®), and darolutamide (Nubeqa®). There is also a class of steroidal antiandrogens, such as cyproterone acetate and spironolactone. Both steroidal and non-steroidal antiandrogens target the LBD of the AR and predominantly fail presumably due to poor affinity and mutations that lead to activation of the AR by these same antiandrogens (Taplin, M. E., Bubley, C. J., Kom Y. J., Small E. J., Uptonm M., Rajeshkumarm B., Balkm S. P., Cancer Res., 59, 2511-2515 (1999)), and constitutively active AR splice variants. Antiandrogens have no effect on the constitutively active AR splice variants that lack the ligand-binding domain (LBD) and are associated with castration-recurrent prostate cancer (Dehm S M, Schmidt L J, Heemers H V, Vessella R L, Tindall D., Cancer Res 68, 5469-77, 2008; Guo Z, Yang X, Sun F, Jiang R, Linn D E, Chen H, Chen H, Kong X. Melamed J, Tepper C G, Kung 1H J, Brodie A M, Edwards J, Qiu Y., Cancer Res. 69, 2305-13, 2009; Hu et al 2009 Cancer Res. 69, 16-22; Sun et al 2010 J Clin Invest. 2010 120, 2715-30) and resistant to abiraterone and enzalutamide (Antonarakis et al., N Engl J Med. 2014, 371, 1028-38; Scher et al JAMA Oncol. 2016 doi: 10.1001). Conventional therapy has concentrated on androgen-dependent activation of the AR through its C-terminal domain.

Other relevant AR antagonists previously reported (see, WO 2010/000066, WO 2011/082487; WO 2011/082488; WO 2012/145330; WO 2015/031984; WO 2016/058080; and WO 2016/058082) that bind to full-length AR and/or truncated AR splice variants that are currently being developed include: AR degraders such as niclosamide (Liu C et al 2014), galeterone (Njar et al 2015; Yu Z at al 2014), and ARV-330/Androgen receptor PROTAC (Neklesa et al 2016 J Clin Oncol 34 suppl 2S; abstr 267); AR DBD inhibitor VPC-14449 (Dalal K et al 2014 J Biol Chem. 289(38):26417-29; Li H et al 2014 J Med Chem. 57(15):6458-67); antiandrogens apalutamide (Clegg N J et al 2012), ODM-201 (Moilanen A M et al 2015), ODM-204 (Kallio et al J Clin Oncol 2016 vol. 34 no. 2_suppl 230), TAS3681 (Minamiguchi et al 2015 J Clin Oncol 33, suppl 7; abstr 266); and AR NTD inhibitors 3E10-AR441bsAb (Goicochea N L et al 2015), and sintokamide (Sadar et al 2008; Banuelos et al 2016).

The AR-NTD is also a target for drug development (e.g. WO 2000/001813; Myung et al. J. Clin. Invest 2013, 123, 2948), since the NTD contains Activation-Function-1 (AF-1) which is the essential region required for AR transcriptional activity (Jenster et al 1991. Mol Endocrinol. 5, 1396-404). The AR-NTD importantly plays a role in activation of the AR in the absence of androgens (Sadar, M. D. 1999 J. Biol. Chem. 274, 7777-7783; Sadar M D et al 1999 Encocr Relat Cancer. 6, 487-502; Ueda et al 2002 J. Biol. Chem., 277, 7076-7085; Ueda 2002 J. Biol. Chem. 277, 38087-38094; Blaszczyk et al 2004 Clin Cancer Res. 10, 1860-9; Dehm et al 2006 J Biol Chem. 28, 27882-93; Gregory et al 2004 J Biol Chem. 279, 7119-30). The AR-NTD is important in hormonal progression of prostate cancer as shown by application of decoy molecules (Quayle et al 2007. Proc Natl Acad Sci USA. 104,1331-1336).

While the crystal structure has been resolved for the AR C-terminus LBD, this has not been the case for the NTD due to its high flexibility and intrinsic disorder in solution (Reid et al 2002 J. Biol. Chem. 277, 20079-20086) thereby hampering virtual docking drug discovery approaches. Compounds that modulate AR, potentially through interaction with NTD domain, include the bisphenol compounds disclosed in published PCT Nos: WO 2010/000066, WO 2011/082487; WO 2011/082488: WO 2012/145330; WO 2012/139039; WO 2012/145328; WO 2013/028572; WO 2013/028791; WO 2014/1179867; WO 2015/031984; WO 2016/058080; WO 2016/058082; WO 2016/112455; WO 2016/141458; WO 2017/177307; WO 2017/210771; WO 2018/045450; and WO 2020/081999, and which are hereby incorporated by reference in their entireties.

Transcriptionally active androgen receptor plays a major role in CRPC in spite of reduced blood levels of androgen (Karantanos, T. et al Oncogene 2013, 32, 5501-5511; Harris, W. P. et al Nature Clinical Practice Urology, 2009, 6, 76-85). AR mechanisms of resistance to ADT include: overexpression of AR (Visakorpi, T. et al Nature Genetics 1995, 9, 401-406; Koivisto, P. et al Scandinavian Journal of Clinical and Laboratory Investigation Supplementum 1996, 226, 57-63); gain-of-function mutations in the AR LBD (Culig Z. et al Molecular Endocrinology 1993, 7, 1541-1550); intratumoral androgen synthesis (Cai, C. et al Cancer Research 2011, 71, 6503-6513); altered expression and function of AR coactivators (Ueda, T. et al The Journal of Biological Chemistry 2002, 277, 38087-38094; Xu J. et al Nature Reviews Cancer 2009, 9, 615-630); aberrant post-translational modifications of AR (Gioeli D. et al Molecular and Cellular Endocrinology 2012, 352, 70-78; van der Steen T. et al International Journal of Molecular Sciences 2013, 14, 14833-14859); and expression of AR splice variants (AR-Vs) which lack the ligand-binding domain (LED) (Karantanos, T. et al Oncogene 2013, 32, 5501-5511; Andersen R. J. et al Cancer Cell 2010, 17, 535-546; Myung J. K. et al The Journal of Clinical Investigation 2013, 123, 2948-2960; Sun S. et al The Journal of Clinical Investigation 2010, 120, 2715-2730). Anti-androgens such as bicalutamide and enzalutamide target AR LBD, but have no effect on truncated constitutively active AR-Vs such as AR-V7 (Li Y. et al Cancer Research 2013, 73, 483-489). Expression of AR-V7 is associated with resistance to current hormone therapies (Li Y. et al Cancer Research 2013, 73, 483-489; Antonarakis E. S. et al The New England Journal of Medicine 2014, 371, 1028-1038).

While significant advances have been made in this field, there remains a need for unproved treatment for AR-mediated disorders including prostate cancer.

### SUMMARY OF THE INVENTION

The present disclosure relates to pharmaceutical compositions comprising Compound A or a pharmaceutically acceptable salt, solvate, stereoisomer or prodrug thereof, wherein the composition is a solid dispersion.

In some embodiments, the solid dispersion is formed by solvent evaporation, hot-melt extrusion or spray drying dispersion.

In some embodiments, the solid dispersion comprises one or more polymers selected from the group consisting of polyethylene glycol (PEG), polyvinyl pyrrolidone (PVP), polyethyleneoxide (PEO), poly(vinyl pyrrolidone-co-vinyl acetate) (PVP-VA), polymethacrylate, polyoxyethylene alkyl ether, polyoxyethylene-polyoxypropylene block copolymer, polyoxyethylene castor oil, polycaprolactam, polylactic acid, polyglycolic acid, poly(lactic-glycolic)acid, lipid, cellulose, pullulan, dextran, dextran acetate, dextran propionate, dextran succinate, dextran acetate propionate, dextran acetate succinate, dextran propionate succinate, dextran acetate propionate succinate, maltodextrin, hyaluronic acid, polysialic acid, chondroitin sulfate, heparin, fucoidan, pentosan polysulfate, spirulan, hydroxymethyl ethylcellulose, hydroxypropyl methylcellulose (HPMC), methyl cellulose, ethyl cellulose, hydroxyethyl cellulose, hydroxypropyl cellulose, carboxymethyl cellulose, carboxymethyl ethylcellulose (CMEC), sodium carboxymethyl cellulose, cellulose acetate succinate (CAS), methyl cellulose acetate succinate (MCAS), hydroxypropyl methylcellulose acetate succinate (HPMCAS), hydroxypropyl methylcellulose propionate succinate, hydroxypropyl methylcellulose propionate phthalate, cellulose acetate phthalate (CAP), hydroxypropyl methyl cellulose phthalate (HPMCP), hydroxypropyl methylcellulose acetate phthalate (HPMCAP), cellulose acetate trimellitate (CAT), hydroxypropyl methylcellulose acetate trimellitate (HPMCAT), hydroxypropyl methylcellulose propionate trimellitate, methyl cellulose acetate phthalate, hydroxypropyl cellulose acetate phthalate, cellulose acetate terephthalate, cellulose acetate isophthalate, cellulose acetate, cellulose butyrate, cellulose acetate butyrate, starch derivatives such as cyclodextrins (CDs), dextran polymer derivative, poly(methacrylic acid-co-methyl methacrylate) 1:1, poly(methacrylic acid-co-methyl methacrylate) 1:2, poly(methacrylic acid-co-ethyl acrylate) 1:1, and a graft copolymers comprised of polyethylene glycol, polyvinyl caprolactam and polyvinyl acetate, or any combinations thereof. In some embodiments, the solid dispersion comprises one or more polymers selected from the group consisting of polyethylene glycol (PEG), polyvinyl caprolactam, polyvinyl acetate, polyvinyl pyrrolidone (PVP), poly(vinyl pyrrolidone-co-vinyl acetate) (PVP-VA), hydroxypropyl methylcellulose (HPMC), hydroxypropyl methylcellulose acetate succinate (HPMCAS), poly(methacrylic acid-co-methyl methacrylate) 1:1, and poly(methacrylic acid-co-methyl methacrylate) 1:2. In one embodiment, the solid dispersion comprises HPMCAS-H.

In some embodiments, the solid dispersion composition comprises Compound A or a pharmaceutically acceptable salt, solvate, stereoisomer or prodrug thereof in an amount ranging from about 10% to about 80% by weight of the composition. In some embodiments, the composition comprises Compound A or a pharmaceutically acceptable salt, solvate, stereoisomer or prodrug thereof in an amount ranging from about 15% to about 45% by weight of the composition. In some embodiments, the weight ratio of the polymer and Compound A or a pharmaceutically acceptable salt, solvate, stereoisomer or prodrug thereof is about 80:20, about 70:30, about 65:35, about 60:40, about 55:45, about 50:50, or about 25:75.

In some embodiments, the solid dispersion has a D₅₀ particle size in the range of about 30 microns to about 60 microns. In some embodiments, the solid dispersion has a D₅₀ particle size in the range of about 10 microns to about 100 microns. In some, embodiments, the solid dispersion has a Doe particle size in the range of about 40 microns to about 130 microns. In some embodiments, the solid dispersion has a D₉₀ particle size in the range of about 70 microns to about 100 microns. In some embodiments, the solid dispersion has a bulk density in the range of about 0.1 g/mL to about 0.6 g/mL In some embodiments, the solid dispersion has a tap density in the range of about 0.2 g/mL to about 0.7 g/mL.

In some embodiments, the solid dispersion comprises less than about 1 wt % water. In some embodiments, the solid dispersion comprises less than about 0.5 wt % water.

In some embodiments, the solid dispersion exhibits a glass transition temperature (Tg) in the range of about 60° C. to about 180° C. as measured by differential scanning calorimeter. In some embodiments, the solid dispersion exhibits a glass transition temperature (Tg) in the range of about 60° C. to about 90° C. as measured by differential scanning calorimeter. In some embodiments, the solid dispersion exhibits a glass transition temperature (Tg) in the range of about 70° C. to about 80° C. as measured by differential scanning calorimeter.

In some embodiments, the solid dispersion exhibits an X-ray powder diffraction (XRPD) pattern substantially similar to any one of the patterns shown in FIGS. 5, 10, and 12. In some embodiments, the solid dispersion exhibits an XRPD pattern substantially similar to a pattern labeled as SDD-A, SDD-B, SDD-C, SDD-D, or SDD-F in FIG. 5 or a pattern labeled as SDD-H, SDD-I, SDD-J, SDD-N, SDD-O, SDD-O. SDD-P, SDD-Q, or SDD-R in FIG. 12. In some embodiments, the solid dispersion exhibits an XRPD pattern substantially similar to a pattern labeled as SDD-H, SDD-1, SDD-J, SDD-N, SDD-O, SDD-O, SDD-P, SDD-Q, or SDD-R in FIG. 12.

In some embodiments, the solid dispersion exhibits a dissolution profile in intestinal buffer (IB) media substantially similar to any one of the profiles shown in FIGS. 8, 9, 13, and 14. In some embodiments, the solid dispersion exhibits a dissolution profile in intestinal buffer (TB) media substantially similar to the profile labeled as SDD-H, SDD-J, SDD-N, SDD-O, SDD-P, or SDD-Q in FIG. 13. In some embodiments, the solid dispersion exhibits a dissolution profile in intestinal buffer (1B) media substantially similar to any one of the profiles shown in FIG. 14.

In some embodiments, the solid dispersion exhibits a modulated differential scanning calorimetry (mDSC) thermogram substantially similar to the thermogram labeled as SDD-A, SDD-B, SDD-C, SDD-D, or SDD-E in FIG. 6.

In some embodiments of the solid dispersion composition, Compound A or a pharmaceutically acceptable salt, solvate, stereoisomer or prodrug thereof is in a crystalline form. In some embodiments, Compound A or a pharmaceutically acceptable salt, solvate, stereoisomer or prodrug thereof is in art amorphous form.

In some embodiments of the solid dispersion composition, Compound A or a pharmaceutically acceptable salt, solvate, stereoisomer or prodrug thereof is in an amorphous form comprising less than 10% of crystalline form of Compound A or a pharmaceutically acceptable salt, solvate, stereoisomer or prodrug thereof. In some embodiments. Compound A or a pharmaceutically acceptable salt, solvate, stereoisomer or prodrug thereof is in an amorphous form comprising less than 5% of crystalline form of Compound A or a pharmaceutically acceptable salt, solvate, stereoisomer or prodrug thereof.

In some embodiments of the solid dispersion composition, the composition is formulated into a tablet. In some embodiments, the composition is filled inside a capsule.

In some embodiments of the solid dispersion composition in the form of a tablet or a capsule, each tablet or each capsule comprises Compound A or a pharmaceutically acceptable salt, solvate, stereoisomer or prodrug thereof in about 50 mg, about 100 mg, about 150 mg, about 200 mg, or about 250 mg. In some embodiments, each tablet or each capsule comprises Compound A or a pharmaceutically acceptable salt, solvate, stereoisomer or prodrug thereof in about 5 mg and about 1000 mg, or between about 10 mg and about 500 mg, or between about 20 mg and about 250 mg, or between about 30 mg and about 300 mg, or between about 50 mg and about 200 mg.

In some embodiments of the solid dispersion composition in a tablet form, the tablet has an average hardness of about 5 kP to about 35 kP. In some embodiments, the tablet has an average tensile strength of about 1 MPa to about 3 MPa. In some embodiments, the tablet has a friability of no more than 1.0% weight loss at 100 drops. In some embodiments, the tablet has an average disintegration time of less than about 300 seconds in an acidic media. In some embodiments, the tablet comprises a film coating.

In some embodiments of the solid dispersion composition, the composition comprises a pharmaceutically acceptable excipient selected from a filler, disintegrant, glidant, or lubricant.

The present disclosure also relates to an amorphous form of Compound A or a pharmaceutically acceptable salt, solvate, or solvate salt thereof; wherein the amorphous form exhibits an X-ray powder diffraction (XRPD) pattern substantially similar to any one of the patterns shown in FIGS. 5, 10, and 12.

In some embodiments, the amorphous form exhibits an XRPD pattern substantially similar to a pattern labeled as SDD-A, SDD-B, SDD-C, SDD-D, or SDD-E in FIG. 5 or a pattern labeled as SDD-H, SDD-I, SDD-J, SDD-N, SDD-O, SDD-O, SDD-P, SDD-Q. or SDD-R in FIG. 12. In some embodiments, the amorphous form exhibits an XRPD pattern substantially similar to a pattern labeled as SDD-H, SDD-1, SDD-J, SDD-N, SDD-O, SDD-O, SDD-P, SDD-Q, or SDD-R in FIG. 12.

In some embodiments, the amorphous form has a solubility is about 40 μg of Compound A/mL to about 50 μg of Compound A/mL in intestinal buffer (IB) media. In some embodiments, the amorphous form has a solubility is about 45 μg of Compound A/mL in intestinal buffer (IB) media.

In some embodiments, the amorphous form exhibits a glass transition temperature (Tg) in the range of about 60° C. to about 180° C. as measured by differential scanning calorimeter. In some embodiments, the amorphous form exhibits a glass transition temperature (Tg) in the range of about 60° C. to about 90° C. as measured by differential scanning calorimeter. In some embodiments, the amorphous form exhibits a glass transition temperature (Tg) in the range of about 60° C. to about 80° C. as measured by differential scanning calorimeter.

In some embodiments, the amorphous form has a purity in the range of about 80% to about 99%. In some embodiments, the amorphous form has a purity of about 95% or higher. In some embodiments, the amorphous form has a purity of about 99% or higher.

In some embodiments, the amorphous form comprises less than about 10% of crystalline form of Compound A or a pharmaceutically acceptable salt, solvate, stereoisomer or prodrug thereof. In some embodiments, the amorphous form comprises lee than about 5% of crystalline form of Compound A or a pharmaceutically acceptable salt, solvate, stereoisomer or prodrug thereof.

The present disclosure also relates to a pharmaceutical composition comprising an amorphous form of Compound A or a pharmaceutically acceptable salt, solvate, stereoisomer or prodrug thereof and a pharmaceutically acceptable excipient or carrier.

The present disclosure also relates to the amorphous form of Compound A or a pharmaceutically acceptable salt, solvate, or solvate salt thereof which is formulated into a solid dispersion composition. Various embodiments of the solid dispersion composition are as disclosed herein.

The present disclosure also relates to methods for modulating androgen receptor activity, comprising administering any one of the pharmaceutical compositions as disclosed herein to a subject in need thereof. The present disclosure also relates to methods for modulating androgen receptor activity, comprising administering any one of the amorphous forms of Compound A as disclosed herein to a subject in need thereof.

In some embodiments, the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

The present disclosure also relates to methods for treating cancer, comprising administering any one of the pharmaceutical compositions as disclosed herein to a subject in need thereof. The present disclosure also relates to methods for treating cancer, comprising administering any one of the amorphous forms of Compound A as disclosed herein to a subject in need thereof.

In some embodiments, the cancer is selected from prostate cancer, breast cancer, ovarian cancer, bladder cancer, pancreatic cancer, hepatocellular cancer, endometrial cancer, or salivary gland carcinoma. In some embodiments, the cancer is prostate cancer. In some embodiments, the prostate cancer is primary or localized prostate cancer, locally advanced prostate cancer, recurrent prostate cancer, advanced prostate cancer, metastatic prostate cancer, metastatic castration-resistant prostate cancer, and hormone-sensitive prostate cancer. In some embodiments, the prostate cancer is metastatic castration-resistant prostate cancer. In some embodiments, the prostate cancer expresses full-length androgen receptor or truncated androgen receptor splice variant. In some embodiments, the prostate cancer is resistant to enzalutamide monotherapy. In some embodiments, the cancer is breast cancer. In some embodiments, the breast cancer is triple negative breast cancer.

### BRIEF DESCRIPTION OF THE DRAWINGS

FIG. 1 shows X-ray powder diffraction (XRPD) spectrum of crystalline Form A of Compound A.

FIG. 2 shows thermogravimetric analysis (TGA)/differential scanning calorimetry (DSC) thermograms of crystalline Form A of Compound A.

FIG. 3A shows XRPD spectrum overlay of NaCl, amorphous form of Compound A, disordered Form A of Compound A, and disordered form of Compound A.

FIG. 3B shows a temperature modulated DSC thermogram of an amorphous form of Compound A.

FIG. 4 shows solubility of amorphous form of Compound A,

FIG. 5 shows XRPD spectrum overlay of SDD compositions A-E and Form A of Compound A.

FIG. 6 shows modulated DSC thermogram overlay of SDD compositions A-E.

FIG. 7 shows concentration of Compound A as determined by UV-Vis probes for SDD compositions A-F and crystalline Compound A in intestinal buffer (IB) portion of a gastric to IB transfer dissolution test.

FIG. 8A shows a scanning electron microscope image of the particles of SDD composition H. FIG. 8B shows a scanning electron microscope image of the particles of SDD composition I FIG. 8C shows a scanning electron microscope image of the particles of SDD composition J. FIG. 8D shows a scanning electron microscope image of the particles of SDD composition N.

FIG. 9A shows a scanning electron microscope image of the particles of SDD composition O. FIG. 9B shows a scanning electron microscope image of the particles of SDD composition P. FIG. 9C shows a scanning electron microscope image of the particles of SDD composition Q. FIG. 9D shows a scanning electron microscope image of the particles of SDD composition R.

FIG. 10 shows XRPD spectrum overlay of SDD compositions G-M after manufacturing and after being stored at 50° C./75% RH for 1 week.

FIG. 11 shows change in Tg of SDD compositions with HPMCAS-H, Eudragit® L100, or Soluplus® (graft copolymers of polyethyleneglycol, polyvinylcaprolactam, and polyvinylacetate) with % loading of Compound A.

FIG. 12 shows XRPD spectrum overlay of SDD compositions H-J and N-R after manufacturing.

FIG. 13 shows non-sink dissolution profiles of SDD compositions H, J, and N-R in intestinal buffer (IB).

FIG. 14 shows non-sink dissolution profiles of SDD compositions J, N, and O with or without external polymer.

FIG. 15A shows PK profile of Compound A in male SD rats. FIG. 15B shows PK profile of Compound A in male Beagle dogs. FIG. 15C shows PK profile of Compound A in male CD-1 mice.

FIG. 16A shows anti-tumor activity of Compound A in castrated SCID Beige male mice bearing VCaP tumors (n=5-6). FIG. 16B shows mean tumor volume in castrated nude mice bearing enzalutamide resistant patient-derived xenograft (PDX) model HID28 after the oral treatment with Compound A or enzalutamide for 28 days (n=6-7 per group).

FIGS. 17A-17C show change in serum prostate specific antigen (PSA) in subjects who received Compound A at 200 mg/day.

FIG. 18 shows change in serum prostate specific antigen (PSA) in one subject through the subject's history of prostate cancer treatments.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a androgen receptor modulator” refers to one or more androgen receptor modulators or at least one androgen receptor modulator. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present disclosure includes tautomers of any said compounds.

A “prodrug” refers to a derivative of a compound of the present disclosure that will be converted to the compound in vivo. In one embodiment of the present disclosure, a prodrug includes a compound of for example abiraterone having a free hydroxyl group (—OH) that is acetylated (—OCOMe) or acylated at one or more positions.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

The term “combination therapy” refers to a first therapy that includes Compound A in conjunction with a second therapy (e.g., therapy, surgery and/or an additional pharmaceutical agent) useful for treating, stabilizing, preventing, and/or delaying the disease or condition.

Administration in “conjunction with” another therapeutically active agent includes administration in the same or different composition(s) and/or combinations, either sequentially, simultaneously, or continuously, through the same or different routes. In some embodiments, the combination therapy optionally includes one or more pharmaceutically acceptable carriers or excipients, non-pharmaceutically active compounds, and/or inert substances.

The terms “pharmaceutical combination,” “therapeutic combination” or “combination” as used herein, refers to a single dosage form comprising at least two therapeutically active agents, or separate dosage forms comprising at least two therapeutically active agents together or separately for use in a combination therapy. For example, one therapeutically active agent may be formulated into one dosage form and the other therapeutically active agent may be formulated into a single or different dosage forms. For example, one therapeutically active agent may be formulated into a solid oral dosage form whereas the second therapeutically active agent may be formulated into a solution dosage form for parenteral administration, including as a kit, or from two kits.

A “fixed dosage form” as used herein means a dosage formulation in which one or more therapeutically active agents are combined in a single dosage formulation.

A “co-packaged form” as used herein means that the therapeutically active agents are taken together, more than one dosage forms wherein the therapeutically active agents are taken together, or more than one dosage forms wherein the therapeutically active agents are taken separately in two or more pharmaceutical compositions, i.e., such as two or more separate tablets, capsules, gel capsules, pellets, etc, but typically the separate compositions are as a single kit.

As used herein, the term “pharmaceutical composition” refers to a formulation comprising at least one therapeutically active agent and a pharmaceutically acceptable excipient or carrier, A non-limiting example of pharmaceutical compositions includes tablets, capsules, gel capsules, syrup, liquid, gel, suspension, solid dispersion, or combinations thereof.

As used herein, the term “dosage form” refers to one or more pharmaceutical compositions which provides a specific amount of a therapeutically active agent, such as a unit dose. In one embodiment, a dosage form can be provided in one or more pharmaceutical compositions. For example, if a subject is to be administered with 200 mg of a therapeutically active agent at a time (unit dose), a dosage form can comprise two tablets each containing 100 mg of the therapeutically active agent, wherein the two tablets are the same pharmaceutical composition.

As used herein, the term “solid dispersion” is a system in a solid state (as opposed to a liquid or gaseous state) comprising at least two components, wherein one component is dispersed more or less evenly throughout the other component or components (homogenous mix). Generally, a solid dispersion formulation of a therapeutically active agent(s) refers to a dispersion mixture of the therapeutically active agent(s) in an inert carrier. Inert carriers can be a crystalline carrier (such as sugars), a polymeric carrier (such as HPMCAS), or a mixture of surfactants and polymers. Typically, a solid dispersion of a therapeutically active agent increases the surface area of the therapeutically active agent and enhances drug solubility and/or dissolution rate.

As used herein, a “subject” can be a human, non-human primate, mammal, rat, mouse, cow, horse, pig, sheep, goat, dog, cat and the like. The subject can be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, such as prostate cancer, breast cancer, ovarian cancer, bladder cancer, pancreatic cancer, hepatocellular cancer, salivary gland carcinoma, or endometrial cancer, and diagnostic methods for acne, hirsutism, alopecia, benign prostatic hyperplasia, ovarian cysts, polycystic ovary disease, precocious puberty, spinal and bulbar muscular atrophy, or age-related macular degeneration and the clinical delineation of cancer, such as prostate cancer, breast cancer, ovarian cancer, bladder cancer, pancreatic cancer, hepatocellular cancer, salivary gland carcinoma, or endometrial cancer, diagnoses and the clinical delineation of acne, hirsutism, alopecia, benign prostatic hyperplasia, ovarian cysts, polycystic ovary disease, precocious puberty, spinal and bulbar muscular atrophy, or age-related macular degeneration are known to those of ordinary skill in the art.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

The following description includes information that may be useful in understanding the present invention. It is not an admission that any of the information provided herein is prior art or relevant to the presently claimed inventions, or that any publication specifically or implicitly referenced is prior art.

### Pharmaceutical Composition of the Disclosure

The present disclosure relates to pharmaceutical compositions comprising Compound A. In one embodiment, the pharmaceutical composition of the present disclosure is a solid dispersion. In one embodiment, the pharmaceutical composition of the present disclosure is useful for treating various diseases and conditions including, but not limited to, cancer. In one embodiment, the pharmaceutical composition of the present disclosure is useful for treating prostate cancer.

### Compound A

The present disclosure relates to pharmaceutical compositions comprising N-(4-((4-(2-(3-chloro-4-(2-chloroethoxy)-5-cyanophenyl)propan-2-yl)phenoxy)methyl)pyrimidin-2-yl)methanesulfonamideN-(4-((4-(2-(3-chloro-4-(2-chloroethoxy)-5-cyanophenyl)propan-2-yl)phenoxy)methyl)pyrimidin-2-yl)methanesulfonamide (Compound A), or a pharmaceutically acceptable salt, solvate, stereoisomer, or prodrug thereof. Compound A has the following structure:

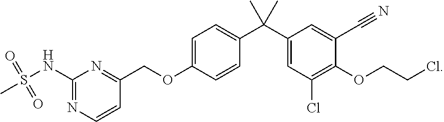 [SMILES: CC(C)(c1ccc(OCc2ccnc(NS(C)(=O)=O)n2)cc1)c1cc(Cl)c(OCCCl)c(C#N)c1]

Compound A is an androgen receptor modulator. Compound A binds to androgen receptor. Specifically, Compound A is an androgen receptor N-terminal domain inhibitor. Related androgen receptor modulators are disclosed in WO2020/081999, which is incorporated by reference in its entirety for all purposes.

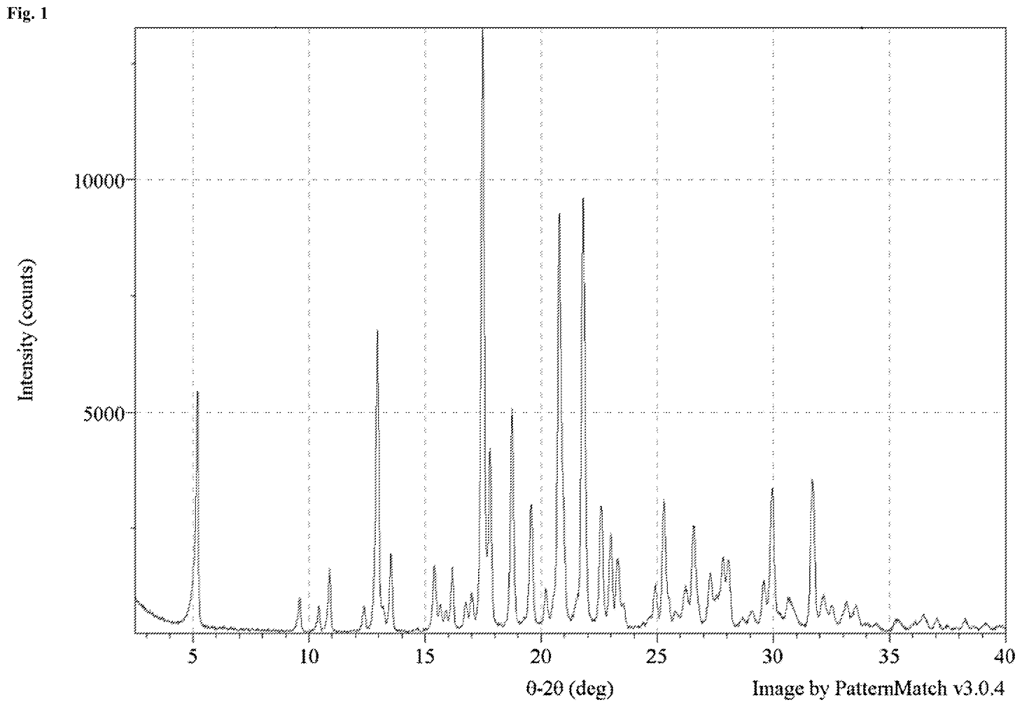

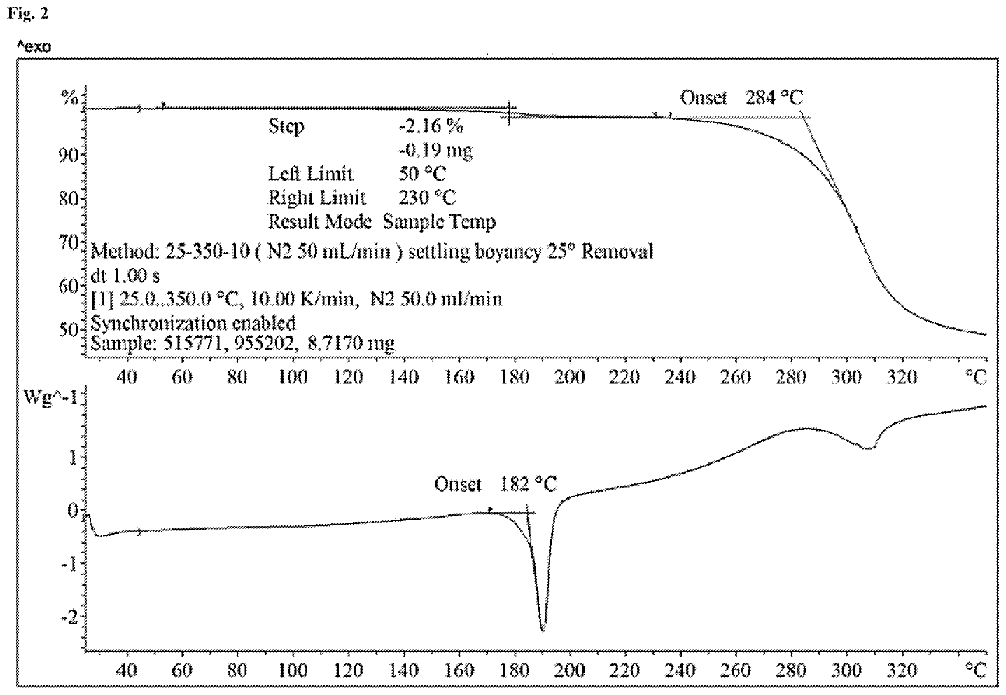

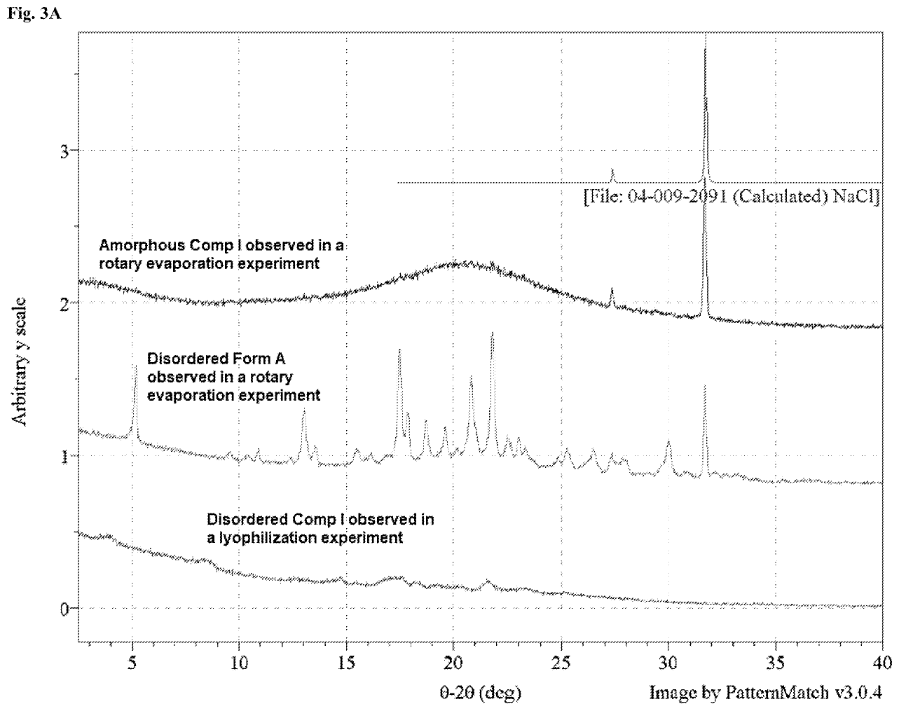

In one embodiment, Compound A or a pharmaceutically acceptable salt, solvate, stereoisomer, or prodrug thereof, in the pharmaceutical composition of the disclosure is in a crystalline form. In one embodiment, Compound A is crystalline Form A. In one embodiment, the crystalline Form A of Compound A exhibits an XRPD pattern that is substantially similar to FIG. 1. In one embodiment, the crystalline Form A of Compound exhibits an XRPD comprising peaks shown in Table 1A. In one embodiment, the crystalline Form A of Compound A exhibits an XRPD comprising peaks shown in Table 1B. In one embodiment, the crystalline Form A of Compound A exhibits a TGA thermogram substantially similar to FIG. 2 (top). In one embodiment, crystalline Form A of Compound A shows change in the slope of a TGA thermogram starting at about 284° C. (onset). In one embodiment, the crystalline Form A of Compound A exhibits a DSC thermogram that is substantially similar to FIG. 2 (bottom).

In one embodiment, Compound A or a pharmaceutically acceptable salt, solvate, stereoisomer, or prodrug thereof, is in an amorphous form.

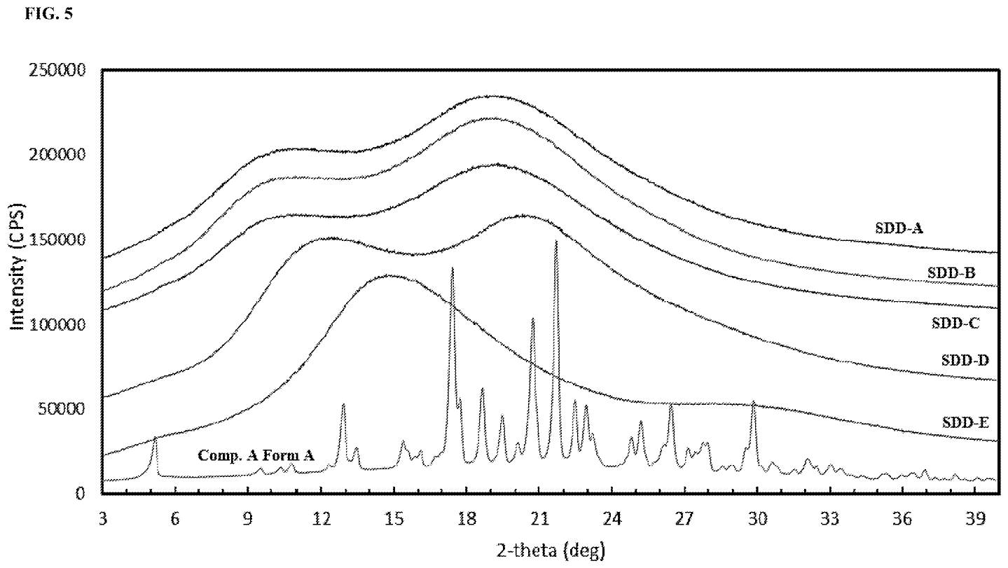

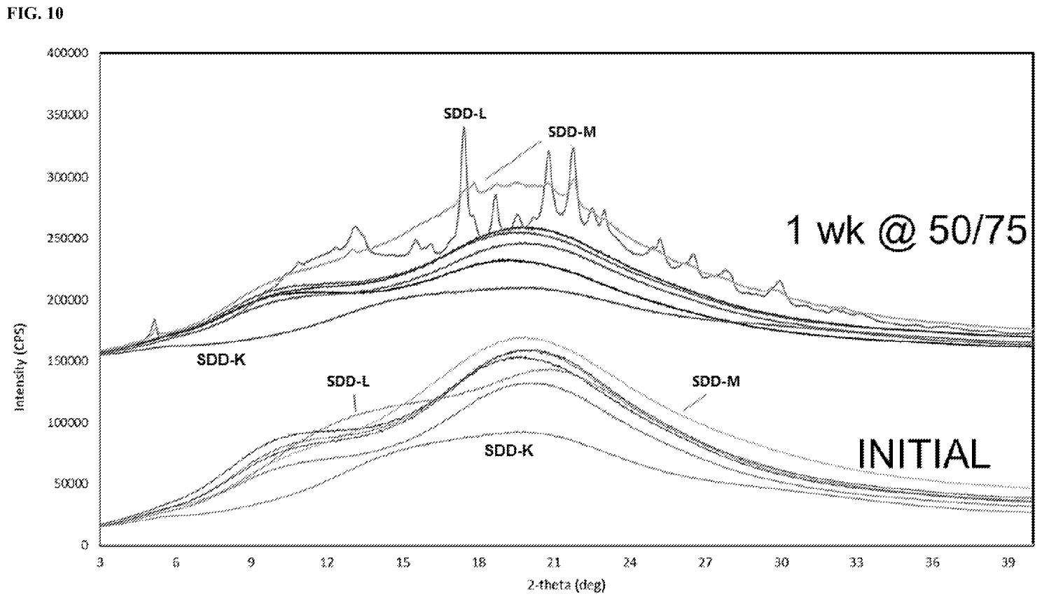

In one embodiment, the present disclosure relates to an amorphous form of Compound A or a pharmaceutically acceptable salt, solvate, or solvate salt thereof; wherein the amorphous form exhibits an X-ray powder diffraction (XRPD) pattern substantially similar to any one of the patterns shown in FIGS. 5, 10 (bottom, ones labeled as “initial”), and 12.

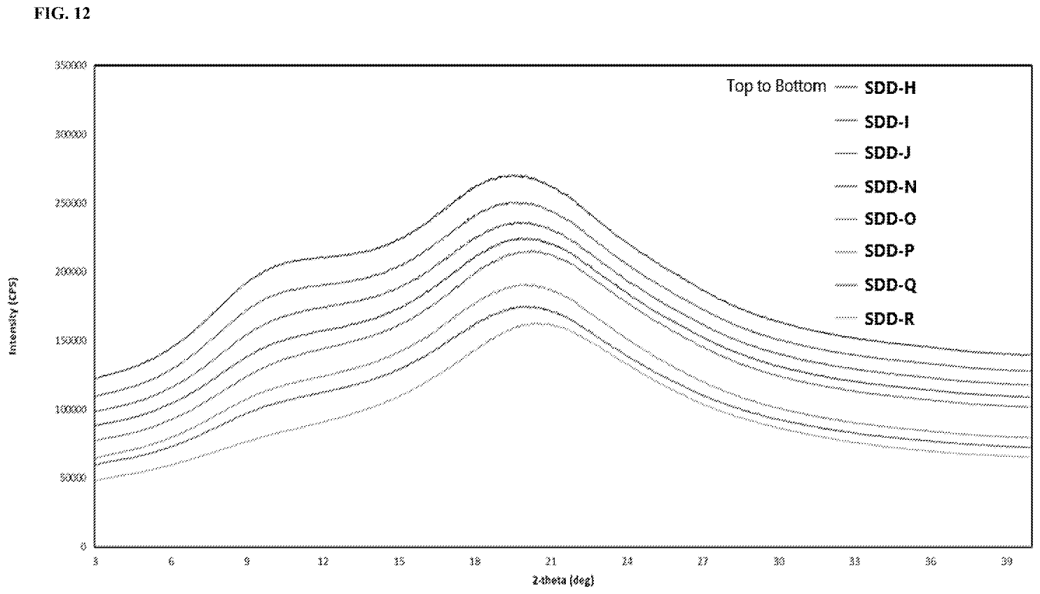

In some embodiments, the amorphous form exhibits an XRPD pattern substantially similar to a pattern labeled as SDD-A, SDD-B, SDD-C, SDD-D, or SDD-E in FIG. 5 or a pattern labeled as SDD-H, SDD-I, SDD-J, SDD-N, SDD-O, SDD-O, SDD-P, SDD-Q, or SDD-R in FIG. 12. In some embodiments, the amorphous form exhibits an XRPD pattern substantially similar to a pattern labeled as SDD-A, SDD-B. SDD-C, SDD-D, or SDD-F in FIG. 5. In some embodiments, the amorphous form exhibits an XRPD pattern substantially similar to a pattern labeled as SDD-H, SDD-I, SDD-J, SDD-N, SDD-O, SDD-O. SDD-P, SDD-Q, or SDD-R in FIG. 12.

In some embodiments, the amorphous form has a solubility is about 40 μg of Compound A/mL (μgA/mL) to about 50 μgA/mL in intestinal buffer (IB) media. In some embodiments, the amorphous form has a solubility is about 45 μgA/mL in intestinal buffer (IB) media. In some embodiments, the amorphous form has a solubility is about 35 μgA/mL, about 36 μgA/mL, about 37 μgA/mL, about 38 μgA/mL, about 39 μg A/mL, about 40 μgA/mL, about 41 μgA/mL, about 42 μgA/mL, about 43 μgA/mL, about 44 μgA/mL, about 45 μgA/mL, about 46 μgA/mL, about 47 μgA/mL, about 48 μgA/mL, about 49 μgA/mL, about 50 μgA/mL, about 51 μgA/mL, about 52 μgA/mL, about 53 μgA/mL, about 54 μgA/mL, or about 55 μgA/mL in TB media. In some embodiments, the solubility is measured in a non-sink dissolution test. In one embodiment, IB media has a pH of 6.5. In one embodiment, IB media is a 0.5% wt simulated intestinal fluid (SIF) in a pH 6.5 phosphate buffer saline (PBS).

In some embodiments, the amorphous form has a solubility is at least about 2.5 μg of Compound A/mL (μgA/mL) in pH 6.5 phosphate buffer saline (PBS). In some embodiments, the amorphous form has a solubility is greater than about 2.5 μg of Compound A/mL (μgA/mL) in pH 6.5 phosphate buffer saline (PBS).

In some embodiments, the amorphous form exhibits a glass transition temperature (Tg) in the range of about 60° C. to about 180° C. as measured by differential scanning calorimeter. In some embodiments, the amorphous form exhibits a glass transition temperature (Tg) in the range of about 60° C. to about 90° C. as measured by differential scanning calorimeter. In some embodiments, the amorphous form exhibits a glass transition temperature (Tg) in the range of about 60° C. to about 80° C. as measured by differential scanning calorimeter. In one embodiment, the amorphous form exhibits a glass transition temperature (Tg) of about 55° C. to about 180° C., about 60° C. to about 170° C., about 60° C. to about 160° C., about 60° C. to about 150° C., about 60° C. to about 140° C., about 60° C. to about 130° C., about 60° C. to about 120° C., about 60° C. to about 110° C., about 60° C. to about 100° C., about 60° C. to about 95° C., or about 60° (C to about 85° C., including all values and subranges therebetween. In one embodiment, the amorphous form exhibits a glass transition temperature (Tg) of about 55° C., about 56° C. t about 57° C. t about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C. as measured by differential scanning calorimeter, including all values therebetween. In one embodiment, the differential scanning calorimeter is modulated differential scanning calorimeter (mDSC). In some embodiments, Tg is determined under dry conditions (0% RH).

In some embodiments, the amorphous form has a purity in the range of about 80% to about 99.9%. In some embodiments, the amorphous form has a purity in the range of about 80% to about 99%. In some embodiments, the amorphous form has a purity of about 95% or higher. In some embodiments, the amorphous form has a purity of about 99% or higher.

In one embodiment, the amorphous form of Compound A or a pharmaceutically acceptable salt, solvate, or solvate salt thereof has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%.

In some embodiments, the amorphous form comprises less than about 50%, less than about 45%, less than about 40%, less than about 35%, less than about 30%, less than about 25%, less than about 20%, less than about 15%, less than about 10%, or less than about 5% by weight of a crystalline form of Compound A or a pharmaceutically acceptable salt, solvate, stereoisomer, or prodrug thereof. In some embodiments, the amorphous form comprises less than about 10% by weight of a crystalline form of Compound A or a pharmaceutically acceptable salt, solvate, stereoisomer, or prodrug thereof. In some embodiments, the amorphous form comprises less than about 5% by weight of a crystalline form of Compound A or a pharmaceutically acceptable salt, solvate, stereoisomer, or prodrug thereof.

In one embodiment of the pharmaceutical compositions comprising Compound A can further comprise one of more additional therapeutically active agents. In one embodiment, the one or more additional therapeutically active agent is useful for treating cancer, treating symptoms associated with cancer, or treating side effects caused by one or more therapeutically active agents. In one embodiment, the one of more additional therapeutically active agents is a metabolic inhibitor that may be beneficial to alter the dosing frequency or dosing amounts of Compound A and/or other therapeutically active agents present in the composition.

In one embodiment, the one or more additional therapeutically active agent is an androgen receptor ligand-binding domain inhibitor, a steroid, a CYP17 inhibitor, a CYP3A4 inhibitor, or an inhibitor of UGT enzymes.

### Androgen Receptor Ligand-Binding Domain Inhibitors

In one embodiment, the additional therapeutically active agent is an androgen receptor ligand-binding domain inhibitor.

In one embodiment, the androgen receptor ligand-binding domain inhibitor is enzalutamide, apalutamide, darolutamide, bicalutamide, nilutamide, flutamide, ODM-204, or TAS3681, or a pharmaceutically acceptable salt or solvate thereof. In one embodiment, the androgen receptor ligand-binding domain inhibitor is enzalutamide, apalutamide, or darolutamide,

### Steroids

In one embodiment, the additional therapeutically active agent is a steroid.

In one embodiment, steroid is aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluocortolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, mometasone, mometasone paramethasone, mometasone furoate monohydrate, prednicarbate, methylprednisolone, prednisolone, prednisone, tixocortol, tixocortol pivalate, triamcinolone, triamcinolone acetonide, or triamcinolone alcohol, or a pharmaceutically acceptable salt or solvate thereof. In one embodiment, steroid is prednisone, prednisolone, or methylprednisolone.

### CY17 Inhibitors

In one embodiment, the additional therapeutically active agent is a CYP17 inhibitor.

In one embodiment, the CYP17 inhibitor is galeterone, abiraterone, or abiraterone acetate, or a pharmaceutically acceptable salt, solvate, or prodrug thereof. In one embodiment, abiraterone prodrug is abiraterone acetate. In one embodiment, the CYP17 inhibitor is abiraterone or abiraterone acetate.

### CYP3A4 Inhibitors

In one embodiment, the additional therapeutically active agent is a CYP3A4 inhibitor.

In one embodiment, the CYP3A4 inhibitor is clarithromycin, telithromycin, erythromycin, nefazodone, atazanavir, darunavir, indinavir, lopinavir, nelfinavir, saquinavir, tipranavir, ritonavir, ketoconazole, itraconazole, fluconazole, verapamil, or cobicistat, or a pharmaceutically acceptable salt or solvate thereof.

### Inhibitors of UGT Enzymes

In one embodiment, the additional therapeutically active agent is an uridine 5′-diphospho-glucuronosyltransferase (UDP-glucurono-syltransferase, UGT) inhibitor.

In one embodiment, the inhibitor of UGT enzyme is atazanavir, indinavir, lopinavir, nelfinavir, ritonavir, saquinavir, silybin, ritonavir, quinidine, diclofenac, everolimus, gemfibrozil, androsterone, phenylbutazone, ketoconazole, nilotinib, deoxyschisandrin, hecogenin, niflumic acid, efavirenz, or amitriptyline, or a pharmaceutically acceptable salt or solvate thereof.

### Solid Dispersion Compositions

The present disclosure relates to pharmaceutical compositions comprising Compound A, wherein the composition is a solid dispersion. In one embodiment, the solid dispersion is formed by solvent evaporation (also known as solvent processing), hot-melt extrusion or spray drying methods. In one embodiment, the solid dispersion is a spray-dried dispersion (SDD).

In one embodiment, the solid dispersion is prepared by solvent evaporation where after the therapeutically active agent and the polymeric carrier are both dissolved in the same solvent or a solvent mixture, the solvent or the solvent mixture is rapidly removed by evaporation or by mixing with a non-solvent. Rapid removal of the solvent or the solvent mixture can be achieved using spray-drying, spray-coating (pan-coating, fluidized bed coating, etc.), and precipitation by rapid mixing of the polymer and drug solution with CO₂, water, or other non-solvent. In one embodiment, the solid dispersion is a supersaturated solid solution where the concentration of the therapeutically active agent in the polymeric carrier is above its equilibrium value.

In one embodiment of the solid dispersion has a single glass transition temperature. A single glass transition temperature for a solid dispersion indicates a high degree of homogeneity.

In one embodiment, the solid dispersion comprises one or more water-soluble polymer. In one embodiment, the solid dispersion comprises one or more cellulose derivatives. In one embodiment, the solid dispersion comprises one or more water-soluble cellulosic polymer. In one embodiment, the solid dispersion comprises one or more cellulosic or non-cellulosic polymer. In one embodiment, the solid dispersion comprises one or more polymer that are neutral or ionizable in aqueous solution. In one embodiment, the solid dispersion comprises one or more polymers that are ionizable and cellulosic. In one embodiment, the solid dispersion comprises one or more polymers that are ionizable cellulosic polymers. In one embodiment, the solid dispersion comprises one or more amphiphilic polymers. In one embodiment, the solid dispersion comprises one or more hydrophilic polymer. In one embodiment, the solid dispersion comprises one or more water-soluble hydrophilic polymer.

In one embodiment, the solid dispersion comprises one or more polymers or polymeric carriers selected from polyethylene glycol (PEG), polyvinyl pyrrolidone (PVP), polyethyleneoxide (PEO), poly(vinyl pyrrolidone-co-vinyl acetate) (PVP-VA), polymethacrylate, polyoxyethylene alkyl ether, polyoxyethylene-polyoxypropylene block copolymer, polyoxyethylene castor oil, polycaprolactam, polylactic acid, polyglycolic acid, poly(lactic-glycolic)acid, lipid, cellulose, pullulan, dextran, dextran acetate, dextran propionate, dextran succinate, dextran acetate propionate, dextran acetate succinate, dextran propionate succinate, dextran acetate propionate succinate, maltodextrin, hyaluronic acid, polysialic acid, chondroitin sulfate, heparin, fucoidan, pentosan polysulfate, spirulan, hydroxymethyl ethylcellulose, hydroxypropyl methylcellulose (HPMC), methyl cellulose, ethyl cellulose, hydroxyethyl cellulose, hydroxypropyl cellulose, carboxymethyl cellulose, carboxymethyl ethylcellulose (CMEC), sodium carboxymethyl cellulose, cellulose acetate succinate (CAS), methyl cellulose acetate succinate (MCAS), hydroxypropyl methylcellulose acetate succinate (HPMCAS), hydroxypropyl methylcellulose propionate succinate, hydroxypropyl methylcellulose propionate phthalate, cellulose acetate phthalate (CAP), hydroxypropyl methyl cellulose phthalate (HPMCP), hydroxypropyl methylcellulose acetate phthalate (HPMCAP), cellulose acetate trimellitate (CAT), hydroxypropyl methylcellulose acetate trimellitate (HPMCAT), hydroxypropyl methylcellulose propionate trimellitate, methyl cellulose acetate phthalate, hydroxypropyl cellulose acetate phthalate, cellulose acetate terephthalate, cellulose acetate isophthalate, cellulose acetate, cellulose butyrate, cellulose acetate butyrate, starch derivatives such as cyclodextrins (CDs), dextran polymer derivative, poly(methacrylic acid-co-methyl methacrylate) 1:1, poly(methacrylic acid-co-methyl methacrylate) 1:2, poly(methacrylic acid-co-ethyl acrylate) 1:1, or a graft copolymers comprised of polyethylene glycol, polyvinyl caprolactam and polyvinyl acetate, or any combinations thereof. In one embodiment, one or more polymers or polymeric carriers is PEG, polyvinyl pyrrolidone, polyethyleneoxide, poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic)acid, lipids, cellulose, pullulan, dextran, maltodextrin, hyaluronic acid, polysialic acid, chondroitin sulfate, heparin, fucoidan, pentosan polysulfate, spirulan, hydroxypropyl methyl cellulose, hydroxypropyl cellulose, 10 carboxymethyl ethylcellulose, hydroxypropyl methylcellulose acetate succinate (including grades L, M, H, LF and/or LG HPMCAS) cellulose acetate phthalate, cellulose acetate trimellitate, ethyl cellulose, cellulose acetate, cellulose butyrate, cellulose acetate butyrate, ethyl cellulose and starch derivatives such as cyclodextrins (CDs), or dextran polymer derivative. In one embodiment, one or more polymers or polymeric carriers is hydroxypropyl methylcellulose acetate succinate. In one embodiment, hydroxypropyl methylcellulose acetate succinate (HPMCAS) has a grade L, M, H, LF, MF, HF. LG, MG, and/or HG, In one embodiment, the polymer or polymeric carrier is HPMCAS-H.

In one embodiment, one or more polymers or polymeric carriers selected from the group consisting of polyethylene glycol (PEG), polyvinyl caprolactam, polyvinyl acetate, polyvinyl pyrrolidone (PVP), poly(vinyl pyrrolidone-co-vinyl acetate) (PVP-VA), hydroxypropyl methylcellulose (HPMC), hydroxypropyl methylcellulose acetate succinate (HPMCAS), poly(methacrylic acid-co-methyl methacrylate) 1:1, and poly(methacrylic acid-co-methyl methacrylate) 1:2.

In one embodiment, a neutral non-cellulosic polymer or polymeric carrier is selected from vinyl polymers and copolymers having substituents of hydroxyl, alkylacyloxy, and cyclicamido polyvinyl alcohols that have at least a portion of their repeat units in the unhydrolyzed (vinyl acetate) form; polyvinyl alcohol polyvinyl acetate copolymers; polyvinyl pyrrolidone; polyvinylpyrrolidone vinyl acetate; or polyethylene polyvinyl alcohol copolymers.

In one embodiment, an ionizable non-cellulosic polymer or polymeric carrier is selected from carboxylic acid-functionalized vinyl polymers, such as the carboxylic acid functionalized polymethacrylates and carboxylic acid functionalized polyacrylates such as the Eudragit® polymers; amine-functionalized polyacrylates and polymethacrylates; proteins; or carboxylic acid functionalized starches such as starch glycolate.

In one embodiment, an amphiphilic non-cellulosic polymer or polymeric carrier is acrylate and methacrylate copolymers. Commercial grades of such copolymers include the Eudragit® polymers, which are copolymers of methacrylates and acrylates; and graft copolymers of polyethyleneglycol, polyvinylcaprolactam, and polyvinylacetate, such as Solupius®.

In one embodiment, a neutral non-ionizable cellulosic polymer or polymeric carrier is selected from hydroxypropyl methyl cellulose acetate, hydroxypropyl methyl cellulose, hydroxypropyl cellulose, methyl cellulose, hydroxyethyl methyl cellulose, hydroxyethyl cellulose acetate, or hydroxyethyl ethyl cellulose.

In one embodiment, a neutral amphiphilic cellulosic polymer or polymeric carrier is selected from hydroxypropyl methyl cellulose or hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

In one embodiment, a cellulosic polymer or polymeric carrier is selected from hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropy cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, or ethyl picolinic acid cellulose acetate. In one embodiment, cellulosic polymers or polymeric carriers are selected from cellulose acetate phthalate (CAP), methyl cellulose acetate phthalate, ethyl cellulose acetate phthalate, hydroxypropyl cellulose acetate phthalate, hydroxylpropyl methyl cellulose phthalate (HPMCP), hydroxypropyl methyl cellulose acetate phthalate (HPMCAP), hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

In one embodiment, a cellulosic ionizable polymer or polymeric carrier is selected from hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, or hydroxyethyl cellulose acetate succinate.

In one embodiment of the pharmaceutical composition of the present disclosure, Compound A or a pharmaceutically acceptable salt, solvate, stereoisomer or prodrug thereof, is present in about 10% to about 90% by weight of the total composition (which can include one or more additional therapeutically active agents and/or other pharmaceutically acceptable excipients and/or coatings), including all values and subranges therebetween. In one embodiment of the solid dispersion, Compound A or a pharmaceutically acceptable salt, solvate, stereoisomer or prodrug thereof, is present in about 10%, about 15%, about 20%, about 25%, about 30%, about 35%, about 40%, about 45%, about 50%, about 55%, about 60%, about 65%, about 70%, about 75%, about 80%, about 85%, or about 90% by weight of the total composition, including all values therebetween. In one embodiment, Compound A or a pharmaceutically acceptable salt, solvate, stereoisomer or prodrug thereof, is present in about 10% to about 80%, about 10% to about 70%, about 10% to about 60%, about 10% to about 50%, about 10% to about 40%, about 10% to about 30%, about 10% to about 20%, about 15% to about 85%, about 15% to about 75%, about 15% to about 65%, about 15% to about 55%, about 15% to about 45%, about 15% to about 35%, about 15% to about 25%, about 20% to about 80%, about 20% to about 70%, about 20% to about 60%, about 20% to about 50%, about 20% to about 40%, about 20% to about 30%, about 25% to about 85%, about 25% to about 75%, about 25% to about 65%, about 25% to about 55%, about 25% to about 45%, about 25% to about 35%, about 30% to about 80%, about 30% to about 70%, about 30% to about 60%, about 30% to about 50%, about 30% to about 40%, about 35% to about 85%, about 35% to about 75%, about 35% to about 65%, about 35% to about 55%, or about 35% to about 45%, by weight of the total composition, including all values therebetween.

In one embodiment of the solid dispersion, the weight ratio of the polymer or the polymeric carrier and Compound A or a pharmaceutically acceptable salt, solvate, stereoisomer or prodrug thereof is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, or about 10:90, including all values therebetween.

In one embodiment, the solid dispersion has a D₅₀ particle size in the range of about 40 microns to about 130 microns, including all values therebetween. In one embodiment, the solid dispersion has a Dao particle size in the range of about 70 microns to about 100 microns, including all values therebetween. In one embodiment, the solid dispersion has a D₅₀ particle size of about 10 microns, about 15 microns, about 20 microns, about 25 microns, about 30 microns, about 35 microns, about 40 microns, about 45 microns, about 50 microns, about 55 microns, about 60 microns, about 65 microns, about 70 microns, about 75 microns, about 80 microns, about 85 microns, about 90 microns, about 95 microns, or about 100 microns, including all values therebetween.

In one embodiment, the solid dispersion has a D₉₀ particle size in the range of about 10 microns to about 100 microns, including all values therebetween. In one embodiment, the solid dispersion has a D₉₀ particle size in the range of about 30 microns to about 60 microns, including all values therebetween. In one embodiment, the solid dispersion has a D₉₀ particle size of about 40 microns, about 45 microns, about 50 microns, about 55 microns, about 60 microns, about 65 microns, about 70 microns, about 75 microns, about 80 microns, about 85 microns, about 90 microns, about 95 microns, about 100 microns, about 105 microns, about 110 microns, about 115 microns, about 120 microns, about 125 microns, or about 130 microns, including all values therebetween.

In one embodiment, the solid dispersion has a bulk density in the range of about 0.1 g/mL to about 0.6 g/mL, including all values therebetween. In one embodiment, the solid dispersion has a bulk density of about 0.1 g/mL, about 0.2 g/mL, about 0.3 g/mL, about 0.4 g/mL, about 0.5 g/mL, or about 0.6 g/mL, including all values therebetween.

In one embodiment, the solid dispersion has a tap density in the range of about 0.2 g/mL to about 0.7 g/mL, including all values therebetween. In one embodiment, the solid dispersion has a tap density of about 0.2 g/mL, about 0.3 g/mL, about 0.4 g/mL, about 0.5 g/mL, about 0.6 g/mL, or about 0.7 g/mL, including all values therebetween.

In one embodiment, the solid dispersion comprises any solvent in less than about 10% by weight. In one embodiment, the solid dispersion comprises any organic solvent in less than about 8% by weight. In one embodiment, the solid dispersion comprises any organic solvent in less than: about 8%, about 7.5%, about 7%, about 6.5%, about 6%, about 5.5%, about 5%, about 4.5%, about 4%, about 3.5%, about 3%, about 2.5%, about 2%, about 1.5%, about 1%, or about 0.5% by weight, including all values therebetween.

In one embodiment, the solid dispersion comprises dichloromethane in less than about 5% by weight. In one embodiment, the solid dispersion comprises dichloromethane in less than about 0.01% by weight. In one embodiment, the solid dispersion comprises dichloromethane in less than: about 5%, about 4.5%, about 4%, about 3.5%, about 3%, about 2.5%, about 2%, about 1.5%, about 1%, about 0.5%, about 0.4%, about 0.3%, about 0.2%, or about 0.1% by weight, including all values therebetween.

In one embodiment, the solid dispersion comprises water in less than about 1% by weight. In one embodiment, the solid dispersion comprises water in less than about 0.5% by weight. In one embodiment, the solid dispersion comprises water in less than: about 1%, about 0.9%, about 0.8%, about 0.7%, about 0.6%, or about 0.5% by weight, including all values therebetween.

In some embodiments, the solid dispersion exhibits a glass transition temperature (Tg) in the range of about 60° C. to about 180° C. as measured by differential scanning calorimeter, including all values therebetween. In some embodiments, the solid dispersion exhibits a glass transition temperature (Tg) in the range of about 60° C. to about 90° C. as measured by differential scanning calorimeter, including all values therebetween. In one embodiment, the solid dispersion exhibits a glass transition temperature (Tg) in the range of about 70° C. to about 80° C. as measured by differential scanning calorimeter, including all values therebetween. In one embodiment, the solid dispersion exhibits a glass transition temperature (Tg) of about 55° C. to about 180° C., about 60° C. to about 170° C., about 60° C. to about 160° C., about 60° C. to about 150° C., about 60° C. to about 140° C., about 60° C. to about 130° C., about 60° C. to about 120° C., about 60° C. to about 110° C., about 60° C. to about 100° C., about 60° C. to about 95° C., or about 60° C. to about 85° C., including all values and subranges therebetween. In one embodiment, the solid dispersion exhibits a glass transition temperature (Tg) of about 70° C., about 71° C., about 72° C., about 73° C., about 74° C., about 75° C., about 76° C., about 77° C., about 78° C., about 79° C., or about 80° C. as measured by differential scanning calorimeter, including all values therebetween. In one embodiment, the differential scanning calorimeter is modulated differential scanning calorimeter (mDSC). Tg is determined under dry conditions (0% RH).

In some embodiments, the solid dispersion exhibits a glass transition temperature (Tg) in the range of about 30° C. to about 100° C. as measured by differential scanning calorimeter under 50% RH, including all values therebetween. In some embodiments, the solid dispersion exhibits a glass transition temperature (Tg) in the range of about 40° C. to about 80° C. as measured by differential scanning calorimeter under 50% RH, including all values therebetween. In some embodiments, solid dispersion exhibits a glass transition temperature (Tg) in the range of about 50° C. to about 70° C. as measured by differential scanning calorimeter under 50% RH, including all values therebetween. In one embodiment, the differential scanning calorimeter is modulated differential scanning calorimeter (mDSC).

In some embodiments, the solid dispersion exhibits a glass transition temperature (Tg) in the range of about 30° C. to about 100° C. as measured by differential scanning calorimeter under 75% RH, including all values therebetween. In some embodiments, the solid dispersion exhibits a glass transition temperature (Tg) in the range of about 40° C. to about 80° C. as measured by differential scanning calorimeter under 75% RI, including all values therebetween. In some embodiments, solid dispersion exhibits a glass transition temperature (Tg) in the range of about 50° C. to about 70° C. as measured by differential scanning calorimeter under 75% RH, including all values therebetween. In one embodiment, the differential scanning calorimeter is modulated differential scanning calorimeter (mDSC).

In some embodiments, the solid dispersion exhibits a glass transition temperature (Tg) in the range of about 30° C. to about 100° C., as measured by differential scanning calorimeter after being stored at 25° C./60% RH for about one month or about two months. In some embodiments, the solid dispersion exhibits a glass transition temperature (Tg) in the range of about 50° C. to about 90° C., as measured by differential scanning calorimeter, after being stored at 25° C./60% RH for about one month or about two months. In some embodiments, the solid dispersion exhibits a glass transition temperature (Tg) in the range of about 60° C. to about 80° C., as measured by differential scanning calorimeter after being stored at 25° C./60% RH for about one month or about two months. In one embodiment, the differential scanning calorimeter is modulated differential scanning calorimeter (mDSC).

In some embodiments, the solid dispersion exhibits a glass transition temperature (Tg) in the range of about 30° C. to about 100° C., as measured by differential scanning calorimeter after being stored at 40° C./75% RH for about one month or about two months. In some embodiments, the solid dispersion exhibits a glass transition temperature (Tg) in the range of about 50° C. to about 90° C., as measured by differential scanning calorimeter, after being stored at 40° C./75%° RH for about one month or about two months. In some embodiments, the solid dispersion exhibits a glass transition temperature (Tg) in the range of about 60° C. to about 80° C., as measured by differential scanning calorimeter after being stored at 40° C./75% RH for about one month or about two months. In one embodiment, the differential scanning calorimeter is modulated differential scanning calorimeter (mDSC).

In some embodiments, the solid dispersion exhibits an X-ray powder diffraction (XRPD) pattern substantially similar to any one of the patterns shown in FIGS. 5, 10, and 12. In some embodiments, the solid dispersion exhibits an XRPD pattern substantially similar to a pattern labeled as SDD-A, SDD-B, SDD-C, SDD-D, or SDD-E in FIG. 5 or a pattern labeled as SDD-H, SDD-I, SDD-J, SDD-N, SDD-O, SDD-O, SDD-P, SDD-Q, or SDD-R in FIG. 12. In some embodiments, the solid dispersion exhibits an XRPD pattern substantially similar to a pattern labeled as SDD-A, SDD-B, SDD-C, SDD-D, or SDD-E in FIG. 5. In some embodiments, the solid dispersion exhibits an XRPD pattern substantially similar to a pattern labeled as SDD-H, SDD-I, SDD-J, SDD-N, SDD-O, SDD-O, SDD-P, SDD-Q, or SDD-R in FIG. 12.

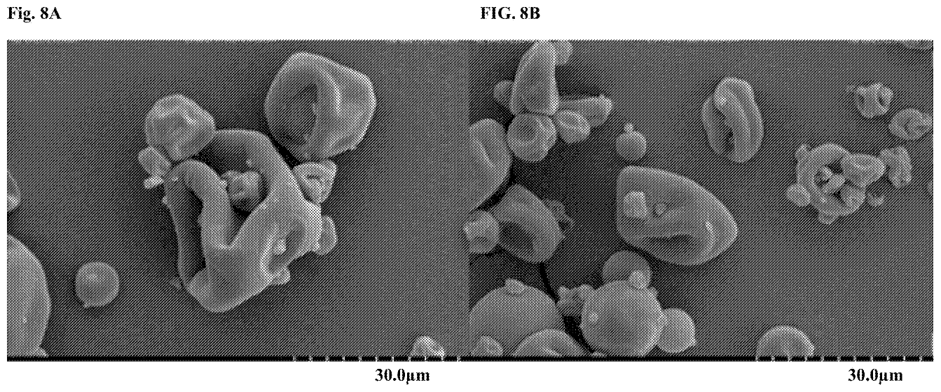

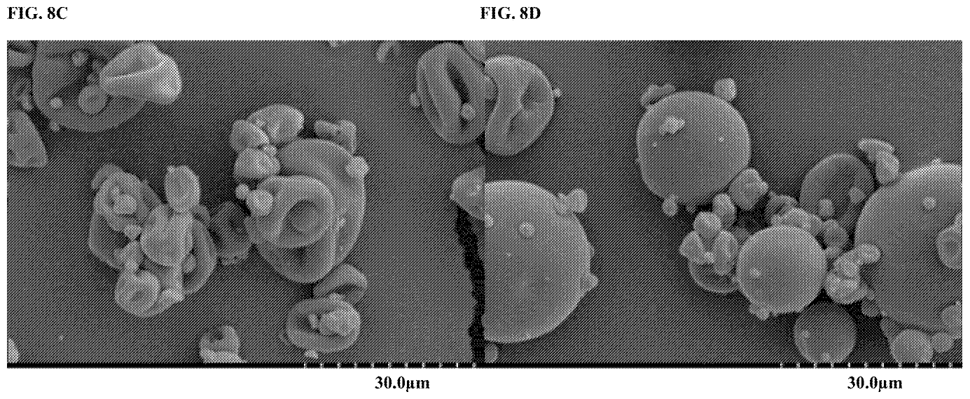

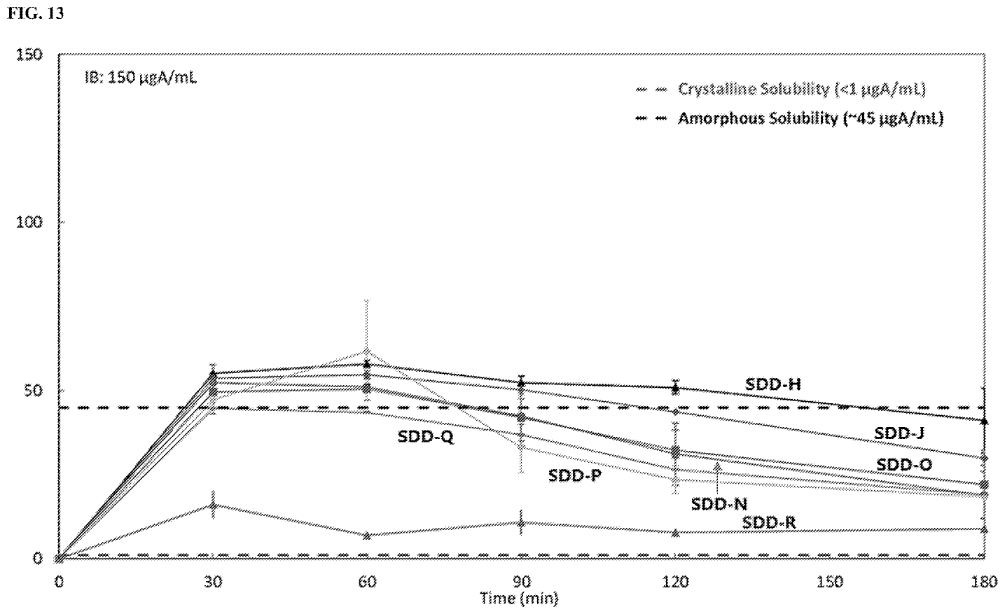

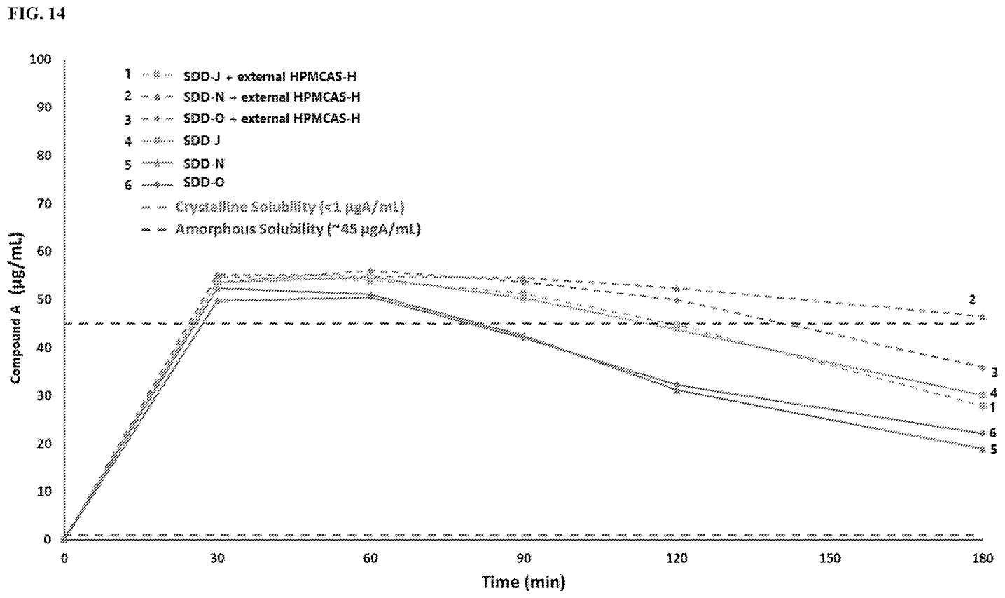

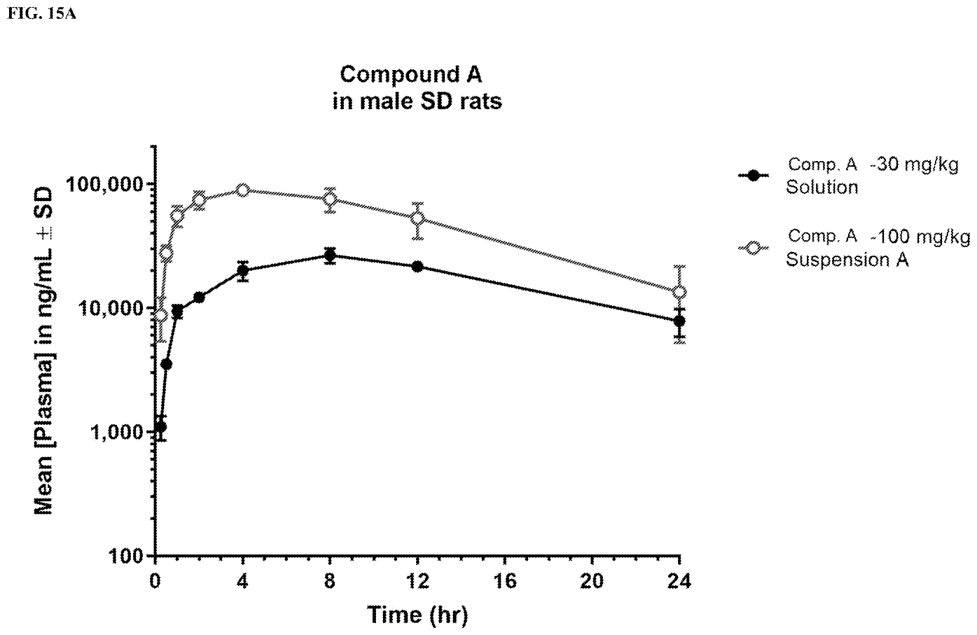

In some embodiments, the solid dispersion exhibits a dissolution profile in intestinal buffer (IB) media substantially similar to any one of the profiles shown in FIGS. 8, 9, 13, and 14. In some embodiments, the solid dispersion exhibits a dissolution profile in intestinal buffer (IB) media substantially similar to the profile labeled as SDD-H, SDD-J, SDD-N, SDD-O, SDD-P, or SDD-Q in FIG. 13. In some embodiments, the solid dispersion exhibits a dissolution profile in intestinal buffer (IB) media substantially similar to any one of the profiles shown in FIG. 14. In some embodiments, the dissolution profile is obtained by a non-sink dissolution test.

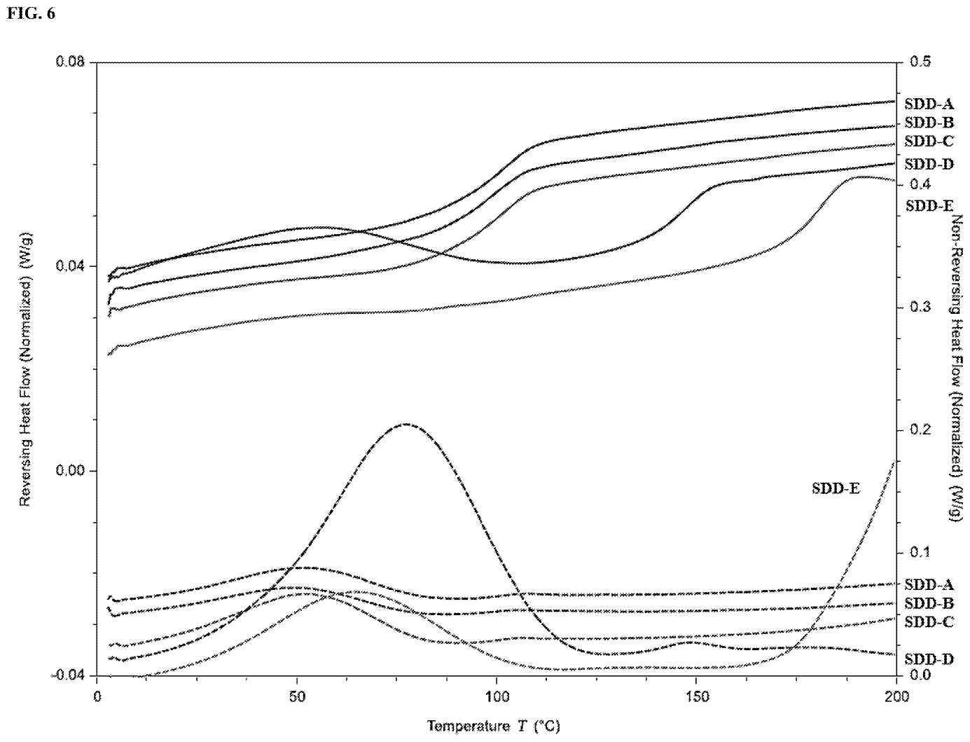

In some embodiments, the solid dispersion exhibits a modulated differential scanning calorimetry (mDSC) thermogram substantially similar to the thermogram labeled as SDD-A, SDD-B, SDD-C, SDD-D, or SDD-E in FIG. 6.

In some embodiments, the solid dispersion reaches a solubility of about 40 pug of Compound A/mL (μgA/mL) to about 50 μgA/mL in intestinal buffer (113) media within about 30 minutes. In some embodiments, the solid dispersion reaches a solubility of about 45 μgA/mL in intestinal buffer (IB) media within about 30 minutes. In some embodiments, the solid dispersion reaches a solubility of about 35 μgA/mL, about 36 μgA/mL, about 37 μgA/mL, about 38 μgA/mL, about 39 μgA/mL, about 40 μgA/mL, about 41 μgA/mL, about 42 μgA/mL, about 43 μgA/mL, about 44 μgA/mL, about 45 μgA/mL, about 46 μgA/mL, about 47 μgA/mL, about 48 μgA/mL, about 49 μgA/mL, about 50 μgA/mL, about 51 μg A/mL, about 52 μgA/mL, about 53 μgA/mL, about 54 μgA/mL, or about 55 μgA/mL in IB media within about 30 minutes. In some embodiments, the solubility is measured in a non-sink dissolution test. In one embodiment, IB media has a pH of 6.5. In one embodiment, IB media is a 0.5% wt simulated intestinal fluid (SIF) in a pH 6.5 phosphate buffer saline (PBS).

In some embodiments, the solid dispersion is physically stable when stored under 25° C./60% RH for about one month. In some embodiments, the solid dispersion is physically stable when stored under 25° C./60% RH for about two months. In some embodiments, the solid dispersion is physically stable when stored under 25° C./60% RH for about three months. In some embodiments, the solid dispersion is physically stable when stored under 25° C./60% RH for up to about three months. In some embodiments, the solid dispersion is physically stable when stored under 25° C./60% RH for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In some embodiment, the solid dispersion is a spray dried dispersion.

In some embodiments, the solid dispersion comprises less than 1 wt % water after being stored at 25° C./60% RH for about one month. In some embodiments, the solid dispersion comprises less than 1 wt % water after being stored at 25° C./60% RH for about two months. In some embodiments, the solid dispersion comprises less than 0.5 wt % water after being stored at 25° C./60% RH for about one month. In some embodiments, the solid dispersion comprises less than 0.5 wt % water after being stored at 25° C./60% RH for about two months. In some embodiments, the solid dispersion comprises less than 1 wt % water or less than 0.5 wt % water after being stored under 25° C./60% RH for at least about 1 month, at least about 2 months, or at least about 3 months. In some embodiments, the solid dispersion gains less than 1 wt % water after being stored at 25° C./60% RH for about one month or about two months. In some embodiments, the solid dispersion gains less than 0.5 wt % water after being stored at 25° C./60% RH for about one month or about two months. In some embodiments, the solid dispersion gains less than 0.3 wt % water after being stored at 25° C./60% RH for about one month or about two months. In some embodiments, the solid dispersion gains less than 1 wt %, less than 0.5 wt %, or less than 0.3% water after being stored under 25° C./60% RH for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In some embodiment, the solid dispersion is a spray dried dispersion.

In some embodiments, the solid dispersion is physically stable when stored under 40° C./75% RH for about one month. In some embodiments, the solid dispersion is physically stable when stored under 40° C./75% RH for about two months. In some embodiments, the solid dispersion is physically stable when stored under 40° C./75% RH for about three months. In some embodiments, the solid dispersion is physically stable when stored under 40° C./75% RH for up to about three months. In some embodiments, the solid dispersion is physically stable when stored under 40° C./75% RH for at least about 1 month, at least about 2 months, at least about 3 months, at least about 4 months, at least about 5 months, or at least about 6 months. In some embodiment, the solid dispersion is a spray dried dispersion.

In some embodiments, the solid dispersion comprises less than 1 wt % water after being stored at 40° C./75% RH for about one month. In some embodiments, the solid dispersion comprises less than 1 wt % water after being stored at 40° C./75% RH for about two months. In some embodiments, the solid dispersion comprises less than 0.5 wt % water after being stored at 40° C./75% RH for about one month. In some embodiments, the solid dispersion comprises less than 0.5 wt % water after being stored at 40° C./75% RH for about two months. In some embodiments, the solid dispersion comprises less than 1 wt % water or less than 0.5 wt % water after being stored under 40° C./75% RH for at least about 1 month, at least about 2 months, or at least about 3 months. In some embodiments, the solid dispersion gains less than 1 wt % water after being stored at 40° C./75% RH for about one month or about two months. In some embodiments, the solid dispersion gains less than 0.5 wt % water after being stored at 40° C./75% RH for about one month or about two months. In some embodiments, the solid dispersion gains less than 0.3 wt % water after being stored at 40° C./75% RH for about one month or about two months. In some embodiments, the solid dispersion gains less than 1 wt %, less than 0.5 wt %, or less than 0.3% water after being stored under 40° C./75% RH for at least about 1 month, at least about 2 months, at least about 3 months, at least about 4 months, at least about 5 months, or at least about 6 months. In some embodiment, the solid dispersion is a spray dried dispersion.

In some embodiments, the solid dispersion has a potency of about 90 mg of Compound A per a gram of the solid dispersion (mgA/g) to about 700 mgA/g, including all values therebetween. In some embodiment, the solid dispersion has a potency of about 100 mgA/g to about 500 mgA/g, including all values therebetween. In some embodiment, the solid dispersion has a potency of about 150 mgA/g to about 400 mgA/g, including all values therebetween. In some embodiment, the solid dispersion has a potency of about 200 mgA/g to about 375 mgA/g, including all values therebetween.

Appropriate form of the pharmaceutical compositions of the present disclosure can be determined according to any clinically-acceptable route of administration of the composition to the subject. The manner in which the composition is administered is dependent, in part, upon the cause and/or location. One skilled in the art will recognize the advantages of certain routes of administration. The method includes administering an effective amount of the agent or compound (or composition comprising the agent or compound) to achieve a desired biological response, e.g., an amount effective to alleviate, ameliorate, or prevent, in whole or in part, a symptom of a condition to be treated, e.g., oncology and neurology disorders. In various aspects, the route of administration is systemic, e.g., oral or by injection. The compositions of the disclosure are administered orally, nasally, transdermally, pulmonary, inhalationally, buccally, sublingually, intraperitoneally, subcutaneously, intramuscularly, intravenously, rectally, intrapleurally, intrathecally, intraportally, and parenterally. Alternatively, or in addition, the route of administration is local, e.g., topical, intra-tumor and peri-tumor. In some embodiments, the composition is administered orally.

In one embodiment of the pharmaceutical composition of the present disclosure, the solid dispersion is formulated into a tablet. In one embodiment, the tablet is in a fixed dosage form.

In one embodiment of the pharmaceutical composition of the present disclosure, a capsule is filled with the solid dispersion. In one embodiment, the capsule is in a fixed dosage form.

In one embodiment of the pharmaceutical composition of the present disclosure, each tablet or capsule comprises Compound A or a pharmaceutically acceptable salt, solvate, stereoisomer or prodrug thereof, in about 5 mg and about 1000 mg, or between about 10 mg and about 500 mg, or between about 20 mg and about 250 mg, or between about 30 mg and about 300 mg, or between about 50 mg and about 200 mg, including all values therebetween.

In one embodiment of the pharmaceutical composition of the present disclosure, each tablet or capsule comprises Compound A or a pharmaceutically acceptable salt, solvate, stereoisomer or prodrug thereof, in about 50 mg, about 100 mg, about 150 mg, about 200 mg, or about 250 mg, including all values therebetween. In one embodiment, an amount of Compound A or a pharmaceutically acceptable salt, solvate, stereoisomer or prodrug thereof, per one tablet or one capsule is about 5 mg, about 10 mg, about 15 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, or any values therebetween.

In one embodiment of the pharmaceutical composition of the present disclosure, an amount of Compound A or a pharmaceutically acceptable salt, solvate, stereoisomer or prodrug thereof, per a dosage form is between about 5 mg and about 1000 mg, or between about 10 mg and about 500 mg, or between about 20 mg and about 250 mg, or between about 30 mg and about 300 mg, or between about 50 mg and about 200 mg, or any values or subranges therebetween. In one embodiment, an amount of Compound A or a pharmaceutically acceptable salt, solvate, stereoisomer or prodrug thereof, per a dosage form is about 5 mg, about 10 mg, about 15 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, or any values therebetween. In one embodiment, an amount of Compound A or a pharmaceutically acceptable salt, solvate, stereoisomer or prodrug thereof, per a dosage form is about 50 mg, about 100 mg, or about 200 mg, or any values therebetween.

In one embodiment of the pharmaceutical compositions of the present disclosure, a daily dosage amount of Compound A or a pharmaceutically acceptable salt, solvate, stereoisomer or prodrug thereof, is between about 50 mg and about 1500 mg, or between about 100 mg and about 1000 mg, or between about 200 mg and about 800 mg, or between about 300 mg and about 600 mg, or any values or subranges therebetween. In one embodiment, the daily dosage amount of Compound A or a pharmaceutically acceptable salt, solvate, stereoisomer or prodrug thereof, is about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 trig, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, or about 1500 mg, or any values therebetween. In one embodiment, the daily dose of Compound A or a pharmaceutically acceptable salt, solvate, stereoisomer or prodrug thereof, is administered once a day, or divided into twice-a-day or three times a day dose. In one embodiment, the daily dose of Compound A or a pharmaceutically acceptable salt, solvate, stereoisomer or prodrug thereof, is provided in one tablet or one capsule, or the daily dose is divided into two, three, four, five, or six tablets or capsules.

The pharmaceutical composition of the present disclosure can further comprise a pharmaceutically acceptable carrier or excipient.

In certain embodiments, a pharmaceutical composition of the present disclosure is prepared for oral administration. In certain of such embodiments, a, pharmaceutical composition is formulated by combining one or more agents and pharmaceutically acceptable carriers. Certain of such carriers enable pharmaceutical compositions to be formulated as tablets, pills, dragees, capsules, gel capsules, liquids, gels, syrups, slurries, suspensions and the like, for oral ingestion by a subject. In some embodiments, the pharmaceutical composition comprises intragranular excipients and extragranular excipients. In some embodiments, extragranular excipients are blended with the solid dispersion prior to being blended with the extragranular excipients.

In some embodiments, the pharmaceutical composition comprises intragranular excipients selected from a filler, a disintegrant, a glidant, and/or a lubricant. In some embodiments, the pharmaceutical composition comprises extragranular excipients selected from a filler, a disintegrant, a glidant, and/or a lubricant.

In one embodiment, the composition can comprise one or more additional therapeutically active agents. In one embodiment, Compound A or a pharmaceutically acceptable salt, solvate, stereoisomer or prodrug thereof, and the additional therapeutically active agent is provided in at least two dosage forms or at least two pharmaceutical compositions. In one embodiment, the at least two dosage forms or the at least two pharmaceutical compositions are co-packaged together into a single kit. In one embodiment, Compound A or a pharmaceutically acceptable salt, solvate, stereoisomer or prodrug thereof, is provided in one dosage form or one pharmaceutical composition and the additional therapeutically active agent is provided in another dosage form or another pharmaceutical composition. In one embodiment, a single kit comprises Compound A or a pharmaceutically acceptable salt, solvate, stereoisomer or prodrug thereof, in one dosage form or a pharmaceutical composition and the additional therapeutically active agent in another dosage form or a pharmaceutical composition. In one embodiment, a single kit comprises Compound A or a pharmaceutically acceptable salt, solvate, stereoisomer or prodrug thereof, formulated into one or more tablets or capsules and the additional therapeutically active agent in different tablets or capsules. In one embodiment, a single kit comprises Compound A or a pharmaceutically acceptable salt, solvate, stereoisomer or prodrug thereof, formulated into one or more tablets and the additional therapeutically active agent in different tablets.

In one embodiment, the tablet is an immediate release tablet. In some embodiment, the tablet has a film coating.

In some embodiments, the tablet has an average hardness of about 10 kP to about 40 kP, including all subranges and values therebetween. In some embodiments, the tablet has an average hardness of about 15 kP to about 35 kP, or about 15 kP to about 30 kP, including all subranges and values therebetween. In some embodiments, the tablet has an average hardness of about 10 kP, about. 11 kP, about 12 kP, about 13 kP, about 14 kP, about 15 kP, about 16 kP, about 17 kP, about 18 kP, about 19 kP, about 20 kP, about 21 kP, about 22 kP, about 23 kP, about 24 kP, about 25 kP, about 26 kP, about 27 kP, about 28 kP, about 29 kP, about 30 kP, about 3l kP, about 32 kP, about 33 kP, about 34 kP, about 35 kP, about 36 kP, about 37 kP, about 38 kP, about 39 kP, about or 40 kP.

In some embodiments, the tablet has art average tensile strength of about 1 MPa to about 5 MPa, including all subranges and values therebetween. In some embodiments, the tablet has an average tensile strength of about 1 MPa, about 1.5 MPa, about 2 MPa, about 2.5 MPa, or about 3 MPa.

In some embodiments, the tablet has a friability of no more than 1.0% weight loss at 100 drops. In some embodiments, the tablet has a friability of no more than about 0.5% weight loss at 100 drops. In some embodiments, the tablet has a friability of no more than about 0.2% weight loss at 100 drops. In some embodiments, the tablet has a friability of no more than about 0.1% weight loss at 100 drops.

In some embodiments, the tablet has a friability of no more than 1.0% weight loss at 300 drops. In some embodiments, the tablet has a friability of no more than about 0.5% weight loss at 300 drops. In some embodiments, the tablet has a friability of no more than about 0.4% weight loss at 300 drops. In some embodiments, the tablet has a friability of no more than about 0.2% weight loss at 300 drops.

In some embodiments, the tablet has a friability of no more than 1.0% weight loss at 500 drops. In some embodiments, the tablet has a friability of no more than about 0.6% weight loss at 500 drops. In some embodiments, the tablet has a friability of no more than about 0.4% weight loss at 500 drops. In some embodiments, the tablet has a friability of no more than about 0.3% weight loss at 500 drops.

In some embodiments, the tablet friability is measured on uncoated tablets (e.g., tablets without film coating or release modifying coating).

In some embodiments, the tablet has an average disintegration time of less than about 300 seconds in an acidic media. In some embodiments, the tablet has an average disintegration time of less than about 250 seconds in an acidic media. In some embodiments, the tablet has an average disintegration time of less than about 200 seconds in an acidic media. In some embodiments, the tablet has an average disintegration time of less than about 160 seconds in an acidic media. In some embodiments, the tablet has an average disintegration time of less than about 100 seconds in an acidic media. In some embodiments, the tablet has an average disintegration time of less than about 60 seconds in an acidic media. In some embodiments, the disintegration test is performed in a USP<701> style basket rack assembly using 0.01N HCl at about 37° C. as the disintegration media.

In one embodiment, a single kit comprises a single dose of Compound A or a pharmaceutically acceptable salt, solvate, stereoisomer or prodrug thereof, and the additional therapeutically active agent. In one embodiment, a single kit comprises a daily dose of Compound A or a pharmaceutically acceptable salt, solvate, stereoisomer or prodrug thereof, and the additional therapeutically active agent. In one embodiment, a daily dose may comprise one or more single doses of Compound A and/or the second therapeutically active agent to be taken at one, two, three, or four different times of the day. In one embodiment, Compound A or a pharmaceutically acceptable salt, solvate, stereoisomer or prodrug thereof, and the additional therapeutically active agent has the same dosing frequency (e.g., once a day, twice a day, once a week), In one embodiment, Compound A or a pharmaceutically acceptable salt, solvate, stereoisomer or prodrug thereof, and the additional therapeutically active agent has the same dosing frequency but taken at different times of the day. In one embodiment, Compound A or a pharmaceutically acceptable salt, solvate, stereoisomer or prodrug thereof, and the additional therapeutically active agent has the same dosing frequency and taken at the same time of the day. In one embodiment, Compound A or a pharmaceutically acceptable salt, solvate, stereoisomer or prodrug thereof, and the additional therapeutically active agent has a different dosing frequency (e.g., Compound A is taken once a day and the additional therapeutically active agent is taken twice a day).

In one embodiment of the pharmaceutical composition of the present disclosure, the combination of Compound A or a pharmaceutically acceptable salt, solvate, stereoisomer or prodrug thereof, and the additional therapeutically active agent is in a single fixed dosage form. In one embodiment, a single tablet or a single capsule fixed dosage form comprises unit doses of Compound A or a pharmaceutically acceptable salt, solvate, stereoisomer or prodrug thereof, and the additional therapeutically active agent. In one embodiment, a single dosage form comprises two or more tablets or capsules, each comprising a fixed-dose of Compound A or a pharmaceutically acceptable salt, solvate, stereoisomer or prodrug thereof, and the second therapeutically active agent.

In one embodiment, the composition of Compound A or a pharmaceutically acceptable salt, solvate, stereoisomer or prodrug thereof, and the additional therapeutically active agent, is provided in at least two dosage forms or at least two pharmaceutical compositions. In one embodiment, the composition is provided in at least three dosage forms. In one embodiment, the at least two dosage forms or the at least two pharmaceutical compositions are co-packaged together into a single kit. In one embodiment, Compound A or a pharmaceutically acceptable salt, solvate, stereoisomer or prodrug thereof, is provided in one dosage form or one pharmaceutical composition and the additional therapeutically active agent is provided in another dosage form or another pharmaceutical composition.

In one embodiment, a single kit comprises Compound A or a pharmaceutically acceptable salt, solvate, stereoisomer or prodrug thereof, in one dosage form or a pharmaceutical composition and the additional therapeutically active agent in another dosage form or a pharmaceutical composition. In one embodiment, a single kit comprises Compound A or a pharmaceutically acceptable salt, solvate, stereoisomer or prodrug thereof, and the second therapeutically active agent formulated into one or more tablets or capsules.

In one embodiment of the pharmaceutical composition of the present disclosure, the pharmaceutical composition can comprise a kit comprising, one, two or three different dosage forms co-packaged together. Different dosage forms in a single co-package can comprise different therapeutically active agents. In some embodiments, all therapeutically active agents (Compound A or a pharmaceutically acceptable salt, solvate, stereoisomer or prodrug thereof, the second, optionally the third, optionally the fourth therapeutically active agents, and so forth) are provided in different dosage forms. In some embodiments, two or more therapeutically active agents are formulated into the same dosage form. In one embodiment, the kit can comprise 1, 2, 3, 4, 5, or 6 pharmaceutical compositions of each dosage form.

In one embodiment of the pharmaceutical composition of the present disclosure, all pharmaceutical compositions are co-packaged for daily administration.

In one embodiment of the pharmaceutical composition of the present disclosure, each pharmaceutical composition of each dosage form is for administration to a subject once every 24 hours, once every 12 hours, once every 8 hours, once every 6 hours, once every 5 hours, or once every 4 hours. In one embodiment, different therapeutically active agents can have different dosing schedule.

In one embodiment of the pharmaceutical compositions of the present disclosure, Compound A and the additional therapeutically active agent are in different compositions but provided in a single kit. In one embodiment, the kit comprises 1, 2, 3, 4, 5, or 6 compositions for each therapeutically active agent to be administered per day. In one embodiment, the kit comprises 1, 2, 3, 4, 5, or 6 tablets or capsules or mixtures of tablets and capsules for each therapeutically active agent.

In one embodiment of the pharmaceutical composition of the present disclosure, at least one composition is a tablet. In one embodiment, Compound A or a pharmaceutically acceptable salt, solvate, stereoisomer or prodrug thereof, and the additional therapeutically active agent are in different layers or compartments of the same tablet composition. In one embodiment, the layers or compartments comprising the different therapeutically active agent is adjacent to one another. In one embodiment, the layers or compartments comprising the different therapeutically active agent are separated by one or more coatings or compartments such that the two therapeutically active agents do not come in contact. The one or more coatings or compartments separating the different therapeutically active agents can be functional (e.g., modifies release) or inert (e.g., just providing physical separation). In one embodiment, the Compound A layer or the Compound A compartment is about 10% to about 70% by weight of the composition, or about 20% to about 50% by weight of the composition, or about 30% to about 40% by weight of the composition, or any values or subranges therebetween. In one embodiment, the Compound A layer or the Compound A compartment is about 10%, about 15%, about 20%, about 25%, about 30%, about 35%, about 40%, about 45%, about 50%, about 55%, about 60%, about 65%, or about 70% weight of the composition, or any values therebetween. In one embodiment, the layer or the compartment of the composition comprising the second therapeutically active agent is about 10% to about 70% by weight of the composition or about 20% to about 50% by weight of the composition, or about 30% to about 40% by weight of the composition, or any values or subranges therebetween. In one embodiment, the layer or the compartment of the composition comprising the second therapeutically active agent is about 10%, about 15%, about 20%, about 25%, about 30%, about 35%, about 40%, about 45%, about 50%, about 55%, about 60%, about 65%, or about 70% weight of the composition, or any values therebetween.

In one embodiment of the pharmaceutical compositions of the present disclosure, a daily dosage amount of the additional therapeutically active agent is about 25 mg to about 550 mg, or about 50 mg to about 480 mg, or about 100 mg to about 400 mg, or about 200 mg to about 300 mg, or any values or subranges therebetween. In one embodiment, an amount of the additional therapeutically active agent per a dosage form is about 5 mg to about 300 mg, or about 10 mg to about 200 mg, or about 30 mg to about 450 mg, or about 200 mg to about 300 mg, or any values or subranges therebetween. In one embodiment, the additional therapeutically active agent is an AR LBD inhibitor. In one embodiment of the pharmaceutical compositions of the present disclosure, the additional therapeutically active agent is enzalutamide, apalutamide, darolutamide, abiraterone, abiraterone acetate, methylprednisolone, or prednisone. In one embodiment, the additional therapeutically active agent is enzalutamide.

### Various Embodiments of Pharmaceutical Compositions, Formulations, Dosage Forms

The pharmaceutical composition as disclosed herein, can further comprise a pharmaceutically acceptable carrier or excipient.

In a further embodiment of the present disclosure, a pharmaceutical composition as disclosed herein comprises a pharmaceutically acceptable carrier, excipient or adjuvant is provided. The pharmaceutically acceptable carriers, excipients and adjuvants are added to the composition or formulation for a variety of purposes. In one embodiment, a pharmaceutically acceptable carrier includes a pharmaceutically acceptable excipient, binder, and/or diluent. In one embodiment, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrolidone.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, PA.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05 M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Pinger's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and may make a pharmaceutical dosage form containing the composition easier for the patient and care giver to handle. Diluents for solid compositions include, for example, microcrystalline cellulose (e.g., AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. EUDRAGIT®), potassium chloride, powdered cellulose, sodium chloride, sorbitol, and talc.

Additional embodiments relate to the pharmaceutical formulations wherein the formulation is selected from the group consisting of a solid, powder, liquid and a gel. In certain embodiments, a pharmaceutical composition of the present invention is a solid (e.g., a powder, tablet, a capsule, granulates, and/or aggregates). In certain of such embodiments, a solid pharmaceutical composition comprising one or more ingredients known in the art, including, but not limited to, starches, sugars, diluents, granulating agents, lubricants, binders, and disintegrating agents.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g., Carbopol®), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL™) hydroxypropyl methyl cellulose (e.g., METHOCEL™), liquid glucose, magnesium aluminum silicate, maltodextrin, methylcellulose, polymethacrylates, povidone (e.g., KOLLIDON®, PLASDONE™), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g., AC-DI-SOL® and PRIMELLOSE®), colloidal silicon dioxide, croscarmellose sodium, crospovidone (e.g., KOLLIDON® and POLYPLASDONE™), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB®), potato starch, and starch. In some embodiments, the disintegrant is crospovidone or arboxymethylcellulose sodium.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate. In some embodiments, the lubricant is magnesium stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid pharmaceutical compositions can optionally have different types of coating. Coatings can be applied to the entire dosage form (e.g. a tablet) or a component of a dosage form (e.g., core, granules, beads, pellets, microparticles, etc). A coating can be used to improve patient compliance (e.g., taste-masking coating, flavor coating, coating to provide smooth surface for easy swallowing), to improve the stability of the compositions (e.g., protection from light, moisture, gas, acid protection, or to divide different layers or compartments to avoid a drug from interacting with different ingredients in a different layer/compartment), alter release profile of the drug (e.g., enteric coating, pH-dependent polymer coating, etc), or improve cosmetic considerations. In some embodiments, the tablet has a cosmetic coating. In some embodiments, the tablet has a film coating. In some embodiments, the tablet has a coating comprising Opadry®.

A coating can be a thin film-coating comprising one or more polymers or water soluble materials including but are not limited to, hypromellose, macrogol, hydroxyethylcellulose, sodium carboxymethylcellulose, polyethylene glycol, polyvinyl alcohol, and cellulose acetate phthalate. In addition, film coating can comprise one or more pharmaceutically acceptable excipients, including but not limited to titanium dioxide, ferric oxide, coloring agents, talc, or lecithin.

A coating that modifies release of the active ingredient can comprise a pH-dependent polymer (e.g., enteric polymer) or a pH-independent polymer. A release-modifying coating can comprise one or more polymers selected from methacrylic copolymers, aminoalkyl methacrylate copolymers, methacrylate copolymers, or ammonioalkyl methacrylate copolymers. A release-modifying coating can comprise one or more cationic polymer, anionic polymer, or neutral polymer.

Solid and liquid compositions may also be dyed using any pharmaceutically acceptable colorant to improve their appearance and/or facilitate patient identification of the product and unit dosage level.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

Liquid pharmaceutical compositions can be prepared where the solid or amorphous components are dissolved or suspended in a liquid carrier such as water, vegetable oil, alcohol, polyethylene glycol, propylene glycol, or glycerin.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Liquid pharmaceutical compositions can contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition can also contain a buffer such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

In one embodiment, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as flanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline cart be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

In certain embodiments, a pharmaceutical composition of the present invention is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition of the present invention comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80 and 65% w/v polyethylene glycol 300, The proportions of such co-solvent systems may be varied considerably without significantly altering their solubility and toxicity characteristics. Furthermore, the identity of co-solvent components may be varied: for example, other surfactants may be used instead of Polysorbate 80; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

In certain embodiments, a pharmaceutical composition of the present disclosure is prepared for oral administration. In certain of such embodiments, a pharmaceutical composition is formulated by combining one or more agents and pharmaceutically acceptable carriers. Suitable excipients include, but are not limited to, fillers, such as sugars, including lactose, lactose monohydrate, sucrose, mannitol, or sorbitol; cellulose preparations such as, for example, maize starch, wheat starch, rice starch, potato starch, gelatin, gum tragacanth, methyl cellulose, microcrystalline cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added. In some embodiments, the filler is microcrystalline cellulose and/or lactose monohydrate.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

In certain embodiments, a pharmaceutical composition is prepared for transmucosal administration. In certain of such embodiment, penetrants appropriate to the barrier to be permeated are used in the formulation. Such penetrants are generally known in the art.

In certain embodiments, a pharmaceutical composition is prepared for administration by inhalation. Certain of such pharmaceutical compositions for inhalation are prepared in the form of an aerosol spray in a pressurized pack or a nebulizer. Certain of such pharmaceutical compositions comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a pharmaceutical agent of the invention and a suitable powder base such as lactose or starch.

In other embodiments, the compound and the compositions of the present disclosure are administered by the intravenous route. In further embodiments, the parenteral administration may be provided in a bolus or by infusion.

In certain embodiments, a pharmaceutical composition is prepared for rectal administration, such as a suppository or retention enema. Certain of such pharmaceutical compositions comprise known ingredients, such as cocoa butter and/or other glycerides.

In certain embodiments, a pharmaceutical composition is prepared for topical administration. Certain of such pharmaceutical compositions comprise bland moisturizing bases, such as ointments or creams. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, one or more therapeutically active agents, or a pharmaceutically acceptable salt or solvate thereof are formulated as a prodrug. In certain embodiments, upon in vivo administration, a prodrug is chemically converted to the biologically, pharmaceutically or therapeutically more active form. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain embodiments, the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

In various aspects, the androgen receptor modulators in the pharmaceutical composition as disclosed herein can be administered at about 0.001 mg/kg to about 100 mg/kg body weight (e.g., about 0.01 mg/kg to about 10 mg/kg or about 0.1 mg/kg to about 5 mg/kg).

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. The dosage regimen utilizing the compounds of the present invention is selected in accordance with a variety of factors including type, species, age, weight, sex and medical condition of the patient; the severity of the condition to be treated: the route of administration; the renal and hepatic function of the patient; and the particular compound or salt thereof employed. Treatments may be administered daily or more frequently depending upon a number of factors, including the overall health of a patient, and the formulation and route of administration of the selected compound(s). An ordinarily skilled physician or veterinarian can readily determine and prescribe the effective amount of the drug required to prevent, counter or arrest the progress of the condition.

The compounds or pharmaceutical compositions of the present disclosure may be manufactured and/or administered in single or multiple unit dose forms.

### Therapeutic Use

The pharmaceutical compositions of the present disclosure find use in any number of methods. For example, in some embodiments the compounds are useful in methods for modulating androgen receptor (AR). In some embodiments, modulating androgen receptor (AR) activity is in a mammalian cell. In some embodiments, modulating androgen receptor (AR) can be in a subject in need thereof (e.g., a mammalian subject) and for treatment of any of the described conditions or diseases.

In one embodiment, the modulating AR is binding to AR. In other embodiments, the modulating AR is inhibiting AR.

In one embodiment, the modulating AR is modulating AR N-terminal domain (NTD). In one embodiment, the modulating AR is modulating AR NTD and AR ligand-binding domain (LBD). In one embodiment, the modulating AR is binding to AR NTD. In one embodiment, the modulating AR is binding to AR NTD and AR LBD. In other embodiments, the modulating AR is inhibiting AR NTD. In other embodiments, the modulating AR is inhibiting AR NTD and AR LBD. In some embodiments, modulating the AR is inhibiting transactivation of androgen receptor N-terminal domain (NTD).

In one embodiment of the present disclosure, methods for modulating androgen receptor activity, comprising administering any one of the pharmaceutical compositions as disclosed herein, to a subject in need thereof, are provided. In other embodiments, modulating androgen receptor (AR) activity is for treatment of at least one indication selected from the group consisting of, prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, age related macular degeneration, and combinations thereof. For example, in some embodiments, the indication is prostate cancer. In other embodiments, the prostate cancer is primary/localized prostate cancer, locally advanced prostate cancer, recurrent prostate cancer, metastatic prostate cancer, advanced prostate cancer, or metastatic castration-resistant prostate cancer (CRPC), or hormone-sensitive prostate cancer. While in other embodiments, the prostate cancer is androgen dependent prostate cancer. In other embodiments, the spinal and bulbar muscular atrophy is Kennedy's disease.

In one embodiment of the present disclosure, a method of treating a condition associated with cell proliferation in a patient in need thereof is provided. In one embodiment, the present invention provides a method of treating cancer or tumors, comprising administering any one of the pharmaceutical compositions as disclosed herein, to a subject in need thereof. In one embodiment, cancer is selected from prostate cancer, breast cancer, ovarian cancer, bladder cancer, pancreatic cancer, hepatocellular cancer, endometrial cancer, or salivary gland carcinoma.

In one embodiment of the methods of the present disclosure, the method is for treating prostate cancer. In one embodiment, prostate cancer is primary or localized prostate cancer, locally advanced prostate cancer, recurrent prostate cancer, advanced prostate cancer, metastatic prostate cancer, metastatic castration-resistant prostate cancer, and hormone-sensitive prostate cancer. In one embodiment, the prostate cancer is metastatic castration-resistant prostate cancer. In one embodiment, the prostate cancer expresses full-length androgen receptor or truncated androgen receptor splice variant. In one embodiment, the prostate cancer is resistant to enzalutamide monotherapy.

In one embodiment of the methods of the present disclosure, the method is for treating breast cancer. In one embodiment, the breast cancer is triple negative breast cancer.

In one embodiment of the present disclosure, a method of reducing, inhibiting, or ameliorating cell proliferation in a patient in need thereof is provided. In one embodiment, the reducing, inhibiting, or ameliorating in the method disclosed herein, is in vivo. In another embodiment, the reducing, inhibiting, or ameliorating is in vitro.

In one embodiment, the cells in the method disclosed herein, are cancer cells. In one embodiment, the cancer cells are prostate cancer cells. In one embodiment, the prostate cancer cells are cells of primary/localized prostate cancer (newly diagnosed or early stage), locally advanced prostate cancer, recurrent prostate cancer (e.g., prostate cancer which was not cured with primary therapy), metastatic prostate cancer, advanced prostate cancer (e.g., after castration for recurrent prostate cancer), metastatic castration-resistant prostate cancer (CRPC), or hormone-sensitive prostate cancer. In another embodiment, the prostate cancer cells are cells of a metastatic castration-resistant prostate cancer. In other embodiments, the prostate cancer cells are androgen-dependent prostate cancer cells or androgen-independent prostate cancer cells. In one embodiment, the cancer cells are breast cancer cells.

In one embodiment, the condition or disease associated with cell proliferation is cancer. In one embodiment of any one of the methods disclosed herein, the cancer is selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. In one embodiment, the condition or disease is prostate cancer. In one embodiment, prostate cancer is selected from primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In another embodiment, the prostate cancer is a metastatic castration-resistant prostate cancer. In some embodiments, the prostate cancer is an androgen-dependent prostate cancer cells or an androgen-independent prostate cancer. In one embodiment, the condition or disease is breast cancer. In one embodiment, the breast cancer is AR-positive triple negative breast cancer.

In another embodiment of the present disclosure, a method for reducing or preventing tumor growth, comprising contacting tumor cells with a pharmaceutical composition as disclosed herein.

In one embodiment, reducing or preventing tumor growth includes reduction in tumor volume. In one embodiment, reducing or preventing tumor growth includes complete elimination of tumors. In one embodiment, reducing or preventing tumor growth includes stopping or halting the existing tumor to grow. In one embodiment, reducing or preventing tumor growth includes reduction in the rate of tumor growth. In one embodiment, reducing or preventing tumor growth includes reduction in the rate of tumor growth such that the rate of tumor growth before treating a patient with the methods disclosed herein (r1) is faster than the rate of tumor growth after said treatment (r2) such that r1>r2.

In one embodiment, the reducing or preventing in the methods disclosed herein is in vivo. In another embodiment, the treating is in vitro.

In one embodiment, the tumor cell in the method disclosed herein is selected from prostate cancer, breast cancer, ovarian cancer, endometrial cancer, or salivary gland carcinoma. In one embodiment, the tumor cells are prostate cancer tumor cells. In one embodiment, the prostate cancer tumor cells are tumor cells of primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In other embodiments, the prostate cancer is a metastatic castration-resistant prostate cancer. In some embodiments, the prostate cancer is androgen-dependent prostate cancer or androgen-independent prostate cancer. In another embodiment, the tumor cells are is breast cancer tumor cells.

### Therapeutic Use Related to Androgen Receptor Driven Gene Expression

In one embodiment, the present disclosure provides a method for treating a subject having a cancer, comprising, obtaining a sample of the cancer before and/or after treatment of the subject with an androgen receptor modulator.

In one embodiment of the present disclosure, a method of treating a patient with abnormal androgen receptor driven gene activity with androgen receptor modulator alone or in combination with one or more additional therapeutically active agent is provided.

In one embodiment, the present disclosure provides a method for treating a subject having a cancer, comprising, obtaining a sample of the cancer before treatment with an androgen receptor modulator, and determining in the sample, the expression level of an androgen receptor driven genes. In another specific embodiment, after testing the expression level of androgen receptor driven genes, the subject is administered an androgen receptor modulator alone and or in combination with additional therapeutically active agent as disclosed herein. In a specific embodiment, the genes are one or more selected from the group consisting of KLK2. FKBP5, TIPRSS2, KLK3, NCAPD3, NKX3-1, NDRG1, STEAP4, FAM105A. AKAP12, PMEPA1, PLPP1, SNA12, ACSL3. ERRF11, CDC6, ELL2 CENPN, RHOU, EAF2, SGK1, SLC16A6, TIPARP, IGF1R, CCND1, ADAMTS1, and PRR15L.

In one embodiment, the present disclosure provides a method of treating cancer in a subject having abnormal gene expression of one or more androgen receptor driven genes, comprising administering to the subject Compound A or a pharmaceutically acceptable salt, solvate, stereoisomer or prodrug thereof, alone or in combination with at least one additional therapeutically active agent. In one embodiment of any one of the methods disclosed herein, the androgen receptor driven gene is an androgen receptor full-length driven gene. In one embodiment, the androgen receptor driven gene is an androgen receptor V7 driven gene. In one embodiment of any one of the methods disclosed herein, the gene with an abnormal activity is selected from KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1. CDC6, ELL2, CENPN, RHOU, EAF2, SGK1, SLC16A6, TIPARP, IGF1R, CCND1, ADAMTS1, or PRR15L. In one embodiment of the methods disclosed herein, cancer is selected from prostate cancer, breast cancer, ovarian cancer, endometrial cancer, or salivary gland carcinoma. In one embodiment, the cancer is prostate cancer. In one embodiment, the prostate cancer is selected from primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In other embodiments, the prostate cancer is a metastatic castration-resistant prostate cancer. In some embodiments, the prostate cancer is androgen-dependent prostate cancer or androgen-independent prostate cancer. In another embodiment, the cancer is breast cancer.

In one embodiment of any one of the methods as disclosed herein, the at least one additional therapeutically active agent is a nonsteroidal antiandrogen (NSAA). In one embodiment, the at least one additional therapeutically active agent is an AR LBD inhibitor.

In one embodiment, the AR LBD inhibitor is enzalutamide, apalutamide, darolutamide, bicalutamide, nilutamide, flutamide, ODM-204, or TAS3681 In one embodiment, the AR LBD inhibitor is enzalutamide, apalutamide, or darolutamide.

In one embodiment of any one of the methods as disclosed herein, the at least one additional therapeutically active agent is an AR LBD inhibitor, a steroid, a CYP17 inhibitor, a CYP3A4 inhibitor, or an inhibitor of UGT enzymes.

In one embodiment, the present disclosure provides a method for treating a subject having a cancer, comprising, obtaining a sample of the cancer after treatment with an androgen receptor modulator, and determining, in the sample, the expression level of an androgen receptor driven gene, where if the gene expression level, when compared to a reference standard level, is decreased before or after treatment with the androgen receptor modulator, then proceeding with or resuming treatment of the subject with a therapeutically effective amount of Compound A or a pharmaceutically acceptable salt, solvate, stereoisomer or prodrug thereof, alone or in combination with at least one additional therapeutically active agent. In a specific embodiment, the gene is selected from one or more from the group consisting of KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, and PRR15L In one embodiment, an androgen receptor modulator administered before the sample of cancer is obtained can be the same or different from Compound A or a pharmaceutically acceptable salt, solvate, stereoisomer or prodrug thereof.

In one embodiment, the at least one additional therapeutically active agent is an androgen receptor ligand-binding domain inhibitor selected from enzalutamide, apalutamide, darolutamide, bicalutamide, nilutamide, flutamide, ODM-204, or TAS3681. In one embodiment, the androgen receptor ligand-binding domain inhibitor is enzalutamide.

In one embodiment, the combination treatment further comprises one or more steroids. In one embodiment, the steroid is prednisone, prednisolone, or methylprednisolone. In one embodiment, the combination treatment further comprises one or more therapeutically active agents. In one embodiment, the one or more therapeutically active agent is an AR LBD inhibitor, a steroid, a CYP17 inhibitor, a CYP3A4 inhibitor, or an inhibitor of UGT enzymes.

Having now generally described the invention, the same will be more readily understood through reference to the following examples, which are provided by way of illustration and are not intended to be limiting of the present invention.

### EXAMPLES

The disclosure now being generally described, it will be more readily understood by reference to the following examples which are included merely for purposes of illustration of certain aspects and embodiments of the present invention and are not intended to limit the invention.

### Example 1: Synthesis of N-(4-((4-(2-(3-chloro-4-(2-chloroethoxy)-5-cyanophenyl)propan-2-yl)phenoxy)methyl)pyrimidin-2-yl)methanesulfonamideN-(4-((4-(2-(3-chloro-4-(2-chloroethoxy)-5-cyanophenyl)propan-2-yl) phenoxy)methyl)pyrimidin-2-yl)methanesulfonamide (A)

2-chloro-4-(chloromethyl)pyrimidine: To a mixture of 2-chloro-4-methyl-pyrimidine (50.0 g, 398 mmol) and NCS (77.9 g, 583 mmol) in MeCN (250 mL) was added benzoyl benzenecarboperoxoate (28.3 g, 117 mmol) in portions at 20° C. and the mixture was stirred at 100° C. for 16 hrs under N atmosphere. TLC showed most of the starting material consumed and two new spots appeared. The mixture was cooled down to room temperature, poured into water (500 mL) and extracted with EtOAc (200 mL×3). The organic layers were combined and washed with brine (200 mL×2), dried over Na₂SO₄, filtered and concentrated under reduced pressure. The residue was purified by silica gel column chromatography to give 2-chloro-4-(chloromethyl)pyrimidine (22 g, yield: 31.2%) as yellow oil. 1H NMR (400 MHz, CDCl3) δ=8.69 (d, J=5.2 Hz, 1H), 7.54 (d, J=5.0 Hz, 1H), 4.61 (s, 2H).

3-chloro-2-(2-chloroethoxy)-5-(2-(4-((2-chloropyrimidin-4-yl)methoxy)phenyl)propan-2-yl)benzonitrile: To a mixture of 3-chloro-24(2-chloroethoxy)-5-(2-(4-hydroxyphenyl)propan-2-yl)benzonitrile (18.0 g, 51.4 mmol) and 2-chloro-4-(chloromethyl)pyrimidine (10.1 g, 61.7 mmol) in DMF (150 mL) was added Cs₂CO₃ (33.5 g, 103.4 mmol) at 20° C. and the mixture was stirred at the same temperature for 16 hrs. LCMS showed the reaction was completed. The reaction mixture was poured into 1-120 (300 mL) and extracted with EtOAc (150 mL×3). The combined organic layers were washed with brine (150 mL×3), dried over Na₂SO₄, filtered and concentrated under reduced pressure. The residue was purified by silica gel column chromatography to give 3-chloro-2-(2-chloroethoxy)-5-(2-(4-((2-chloropyrimidin-4-yl)methoxy)phenyl)propan-2-yl)benzonitrile (15.5 g, yield: 63.3%) as white solid. ¹H NMR (400 MHz, CDCl3) δ=8.67 (d, J=5.2 Hz, 1H), 7.56 (d, J=5.2 Hz, 1H), 7.45 (d, J=2.4 Hz, 1H), 7.35-7.29 (m, 1H), 7.13 (d, J=8.8 Hz, 21H), 6.90 (d, J=8.8 Hz, 21H), 5.16 (s, 21H), 4.43 (t, J=6.0 Hz, 2H), 3.88 (t, J=6.0 Hz, 2H), 1.65 (s, 6H).

N-(4-((4-(2-(3-chloro-4-(2-chloroethoxy)-5-cyanophenyl)propan-2-yl)phenoxy)methyl)pyrimidin-2-yl)methanesulfonamide (A): To a mixture of 3-chloro-2-(2-chloroethoxy)-5-(2-(4-((2-chloropyrimidin-4-yl)methoxy)phenyl)propan-2-yl)benzonitrile (15.5 g, 32.5 mmol), methane sulfonamide (9.3 g, 97.5 mmol), Cs₂CO₃ (21.2 g, 65.0 mmol) and Xantphos (1.88 g, 3.25 mmol) in 1,4-dioxane (450 mL) was added Pd₂(dba)₃ (3.0 g, 3.3 mmol) at 20° C. and the mixture was stirred at 90° C. for 6 hrs under N₂ atmosphere. LCMS showed the reaction was completed. The mixture was cooled down to room temperature, poured into water (300 mL) and extracted with EtOAc (300 mL×3). The combined organic layers were washed with brine (300 mL×2), dried over Na₂SO₄, filtered and concentrated under reduced pressure. The residue was purified by silica gel column chromatography to give the crude product and then further purified by p-HPLC (TFA) to give N-(4-((4-(2-(3-chloro-4-(2-chloroethoxy)-5-cyanophenyl)propan-2-yl)phenoxy)methyl)pyrimidin-2-yl)methanesulfonamide (5.30 g, yield: 30.1%) as yellow solid. H NMR (400 MHz, CDCl3) δ=10.02 (br s, 1H), 8.69 (d, J=5.2 Hz, 1H), 7.45 (d, J=2.4 Hz, 1H), 7.34-7.31 (m, 1H), 7.30 (d, J=5.2 Hz, 1H), 7.13 (d, J=8.8 Hz, 2H), 6.91 (d, J=8.8 Hz, 2H), 5.13 (s, 2H), 4.43 (t, J=6.0 Hz, 2H), 3.88 (t, J=6.0 Hz, 2H), 3.47 (s, 3H), 1.65 (s, 6H). LCMS (220 nm): 99.0%. Exact Mass: 534.09; found 535.1, 537.0. See WO 2020/081999.

### Example 2: Synthesis and Characterization of Crystalline Form a of Compound A

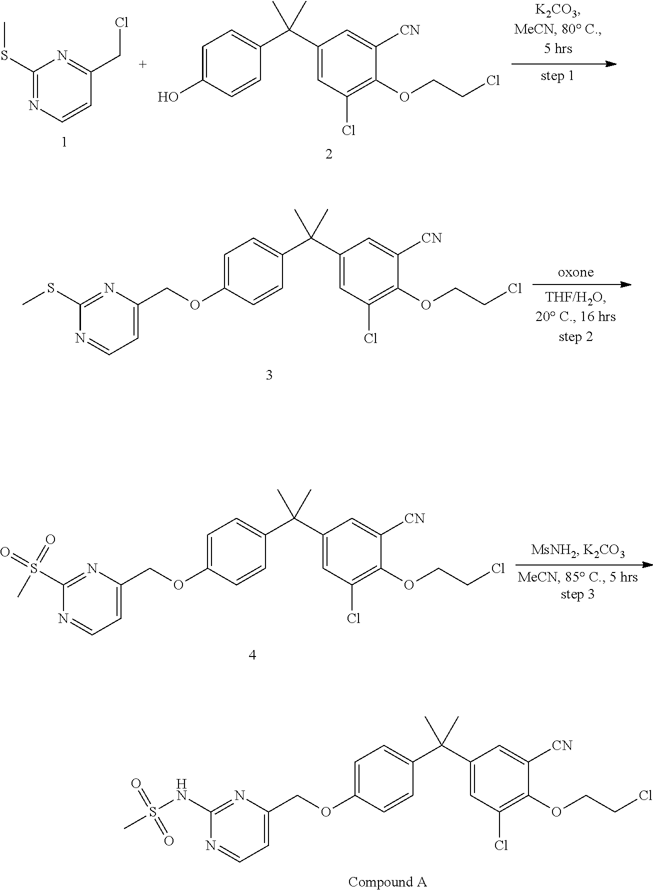

Step 1: A mixture of 4-(chloromethyl)-2-methylsulfanyl-pyrimidine (1) (324 mg, 1.86 mmol), 3-chloro-2-(2-chloroethoxy)-5-(2-(4-hydroxyphenyl)propan-2-yl)benzonitrile (2) (0.5 g, 1.43 mmol) and K₂CO₃ (493 mg, 3.57 mmol) in MeCN (4 mL) was stirred at 80° C. for 5 hrs. LCMS and HPLC showed the reaction was completed and 81.4% of the desired product formed. The resulting mixture was quenched with sat.NH₄Cl (10 mL) and extracted with EtOAc (10 mL×3). The combined organic layers were washed with brine (10 mL), dried over Na₂SO₄, filtered and concentrated under reduced pressure. The residue was purified by MPLC to give 3-chloro-2-(2-chloroethoxy)-5-(2-(4-((2-(methylthio)pyrimidin-4-yl)methoxy)phenyl)propan-2-yl)benzonitrile (3) (0.54 g, yield: 77.4%) as colorless syrup. ¹H NMR (400 MHz, CDCl3) δ=8.54 (d, J=4.8 Hz, 1H), 7.45 (d, J=2.4 Hz, 1H), 7.32 (d, J=2.4 Hz, 1H), 7.22 (d, J=5.2 Hz, 1H), 7.12 (d, J=8.8 Hz, 2H), 6.89 (d, J=8.8 Hz, 2H), 5.09 (s, 2H), 4.43 (t, J=6.4 Hz, 2H), 3.88 (t, J=6.4 Hz, 2H), 2.59 (s, 3H), 1.65 (s, 6H), after work up: HPLC (220 nm): 94.7%. LCMS (220 urn): 93.5%, Exact Mass: 487.1: found 488.0/490.0.

Step 2: To a suspension of 3-chloro-2-(2-chloroethoxy)-5-(2-(4-((2-(methylthio)pyrimidin-4-yl)methoxy)phenyl)propan-2-yl)benzonitrile (3) (1.07 g, 2.19 mmol) in THF (20 mmL) was added a suspension of Oxone (5.39 g, 8.76 mmol) in water (20 mL) at 20° C. The mixture was stirred at 20° C. for 16 hrs. LCMS and HPLC showed the reaction was completed and 93.0% of the desired product formed. The resulting mixture was quenched with sat.Na₂SO₃. The aqueous layer was extracted with EtOAc (30 mL×3). The combined organic layers were washed with brine (30 ml), dried over Na₂SO₄, filtered and concentrated under reduced pressure to give 3-chloro-2-(2-chloroethoxy)-5-(2-(4-((2-(methylsulfonyl)pyrimidin-4-yl)methoxy)phenyl)propan-2-yl)benzonitrile (4) (1.04 g, yield: 91.2%) as colorless syrup. ¹H NMR (400 MHz, CDCl3) δ=8.94 (d, J=4.8 Hz, 1H), 7.85 (d, J=4.8 Hz, 1H), 7.45 (d, J=2.4 Hz, 1H), 7.31 (d, J=2.4 Hz, 1H), 7.15 (d, J=8.8 Hz, 2H) 6.91 (d, J=8.8 Hz, 2H), 5.30 (s, 2H), 4.43 (t, J=6.4 Hz, 2H), 3.88 (t, J=6.0 Hz, 2H), 3.40 (s, 3H), 1.66 (s, 61H). IPC detection: HPLC (220 nm): 92.956%. LCMS (220 nm): 93.0%. Exact Mass: 519.1; found 520.1/522.1.

Step 3: A suspension of 3-chloro-2-(2-chloroethoxy)-5-(2-(4-((2-(methylsulfonyl)pyrimidin-4-yl)methoxy)phenyl)propan-2-yl)benzonitrile (4) (30 mg, 0.058 mmol), methanesulfonamide (11 mg, 0.12 mmol) and K₂CO₃ (15.9 mg, 0.12 mmol) in MeCN (2 mL) was stirred at 85° C. for 5 hrs. LCMS showed the reaction was completed and 91.6% of the desired product formed. The resulting mixture was partitioned between EtOAc (2 mL) and aq.NH₄Cl (2 mL). The aqueous layer was extracted with EtOAc (2 mL×3). The combined organic layers were washed with brine (2 mL), dried over Na₂SO₄, filtered and concentrated under reduced pressure to give Compound A, Form A (40 mg) as yellow solid. ¹H NMR (400 MHz, CDCl3) δ=8.77 (br s, 1H), 8.64 (d, J=4.8 Hz, 1H), 7.45 (d, J=2.4 Hz, 1H), 7.32 (d, J=2.4 Hz, 1H), 7.29 (d, J=5.2 Hz, 1H), 7.13 (d, J=8.8 Hz, 2H), 6.90 (d, J=8.8 Hz, 2H), 5.11 (s, 2H), 4.43 (t, J=6.0 Hz, 2H), 3.88 (t, J=6.0 Hz, 2H), 3.48 (s, 3H), 1.65 (s, 6H). IPC detection: LCMS (220 nm): 91.6% purity. Exact Mass: 534.1; found 535.1/537.2.

XRPD spectrum was obtained for Form A as shown in FIG. 1 and Tables 1A-1B. The sample likely contains small amount of NaCl. The sharp peaks at 27.3±0.2 and at 31.7±0.2 degrees two-theta is consistent with the presence of NaCl. Form A was determined successfully in this study, and the results indicate it is an anhydrous/unsolvated material.

TGA/DSC thermograms were also obtained for Form A as shown in FIG. 2. By TGA, a weight loss of 2.2 wt % was observed from 50-230° C. which is likely due to the loss of residual solvents in the sample. The dramatic change in the slope of the thermogram starting at about 284° C. (onset) is likely associated with the decomposition of the material. By DSC, an endotherm is observed at approximately 182° C. (onset), which could be due to the melting of the material.

TABLE 1A
XRPD Table of Form A of Compound A

| °2θ | d space (Å) | Intensity (%) |
|---|---|---|
| 5.19 ± 0.20 | 17.013 ± 0.655 | 41 |
| 9.60 ± 0.20 | 9.206 ± 0.191 | 8 |
| 10.42 ± 0.20 | 8.483 ± 0.162 | 6 |
| 10.89 ± 0.20 | 8.118 ± 0.149 | 12 |
| 12.38 ± 0.20 | 7.144 ± 0.115 | 6 |
| 12.94 ± 0.20 | 6.836 ± 0.105 | 51 |
| 13,17 ± 0.20 | 6.715 ± 0.101 | 6 |
| 13.52 ± 0.20 | 6.544 ± 0.096 | 15 |
| 15,40 ± 0.20 | 5.749 ± 0.074 | 13 |
| 15.64 ± 0.20 | 5.661 ± 0.072 | 7 |
| 15.90 ± 0.20 | 5.569 ± 0.070 | 5 |
| 16.17 ± 0.20 | 5.477 ± 0.067 | 13 |
| 16.75 ± 0.20 | 5.289 ± 0.063 | 7 |
| 17.01 ± 0.20 | 5.208 ± 0.061 | 8 |
| 17.48 ± 0.20 | 5.069 ± 0.058 | 100 |
| 17.80 ± 0.20 | 4.979 ± 0.055 | 32 |
| 18.74 ± 0.20 | 4.731 ± 0.050 | 38 |
| 19.57 ± 0.20 | 4.532 ± 0.046 | 23 |
| 20.20 ± 0.20 | 4.392 ± 0.043 | 9 |
| 20.78 ± 0.20 | 4.271 ± 0.041 | 70 |
| 21.80 ± 0.20 | 4.074 ± 0.037 | 73 |
| 22.59 ± 0.20 | 3.933 ± 0.034 | 23 |
| 22.99 ± 0.20 | 3.865 ± 0.033 | 18 |
| 23.29 ± 0.20 | 3.816 ± 0.032 | 14 |
| 23.53 ± 0.20 | 3.778 ± 0.032 | 7 |
| 24.91 ± 0.20 | 3.572 ± 0.028 | 10 |
| 25.28 ± 0.20 | 3.520 ± 0.027 | 23 |
| 25.82 ± 0.20 | 3.448 ± 0.026 | 5 |
| 26.21 ± 0.20 | 3.397 ± 0.025 | 9 |
| 26.57 ± 0.20 | 3.352 ± 0.025 | 19 |
| 27.29 ± 0.20 | 3.265 ± 0.023 | 12 |
| 27.83 ± 0.20 | 3.203 ± 0.023 | 14 |
| 28.06 ± 0.20 | 3.177 ± 0.022 | 13 |
| 28.68 ± 0.20 | 3.110 ± 0.021 | 4 |
| 29.09 ± 0.20 | 3.067 ± 0.021 | 5 |
| 29.59 ± 0.20 | 3.016 ± 0.020 | 10 |
| 29.95 ± 0.20 | 2.981 ± 0.019 | 25 |

TABLE 1B
XRPD Table of Form A of Compound A

| °2θ | d space (Å) | Intensity (%) |
|---|---|---|
| 5.19 ± 0.20 | 17.013 ± 0.655 | 41 |
| 12.94 ± 0.20 | 6.836 ± 0.105 | 51 |
| 17.48 ± 0.20 | 5.069 ± 0.058 | 100 |
| 17.80 ± 0.20 | 4.979 ± 0.055 | 32 |
| 18.74 ± 0.20 | 4.731 ± 0.050 | 38 |
| 19.57 ± 0.20 | 4.532 ± 0.046 | 23 |
| 20,78 ± 0.20 | 4.271 ± 0.041 | 70 |
| 21.80 ± 0.20 | 4.074 ± 0.037 | 73 |
| 22.59 ± 0.20 | 3.933 ± 0.034 | 23 |
| 25.28 ± 0.20 | 3.520 ± 0.027 | 23 |
| 29.95 ± 0.20 | 2.981 ± 0.019 | 25 |

### Example 3: Preparation of Amorphous Form of Compound A

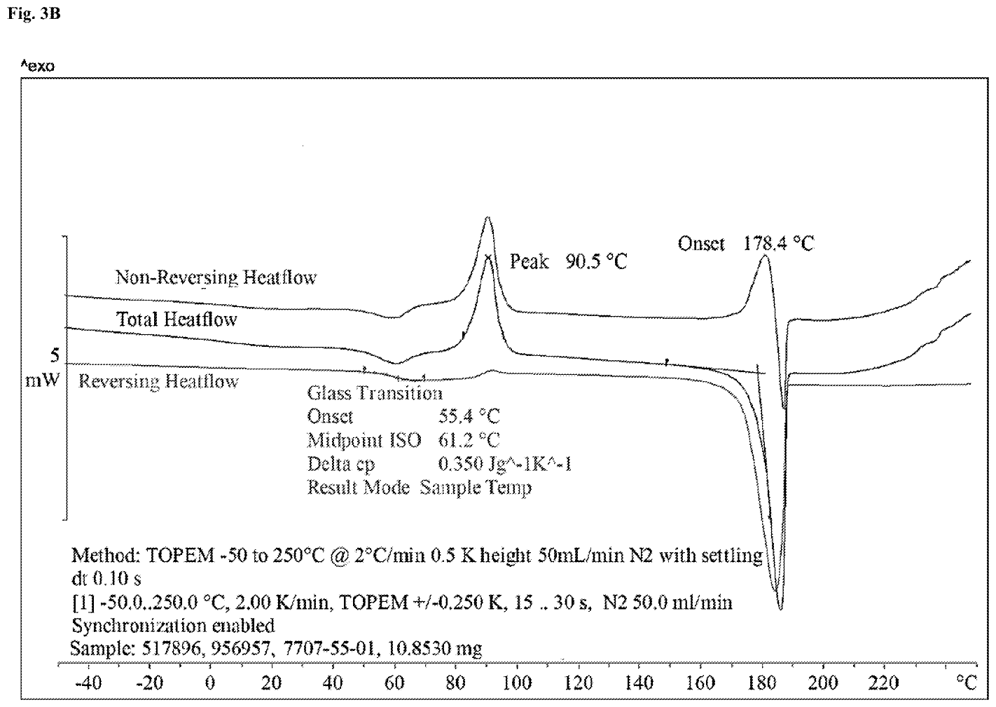

Dilute solutions of Compound A were prepared in DCM and filtered through 0.2-μm nylon filters into a clear round bottom flasks. The flasks were attached to a rotary evaporator and immersed in a water bath at specified temperatures and DCM was rapidly evaporated to dryness under vacuum. The samples underwent secondary drying under vacuum at room temperature for 1 day, then analyzed by XRPD. XRPD, displayed broad halos with crystalline peaks due to NaCl, indicating successful generation of an amorphous form of Compound A (“x-ray amorphous”). See FIG. 3A, third spectrum from bottom.

Materials described as “x-ray amorphous” are typically characterized further by thermal analysis where the appearance of a glass transition (T_{g}) provides support for the non-crystalline nature of the material Temperature modulated DSC was performed on the material to investigate the T_{g} (Table 3). As shown in FIG. 3B, T_{g} was observed at approximately 61° C. as a step change in the reversing heat flow signal. On further heating, an exotherm likely due to crystallization was observed at about 91° C. (peak). The endotherm has a slightly lower temperature than the endotherm observed for Form A (182° C., FIG. 2), which could be due to the specimen containing an amorphous or disordered portion (i.e., not completely crystallized during the analysis).

TABLE 3
Characterization of Amorphous Form of Compound A

| Material | Analysis | Results |
|---|---|---|
| x-ray | 1H NMR | consistent with the chemical |
| amorphous | | structure of Comp. A; contains |
| (w/NaCl) | | trace DCM (~0.004 mol/mol) |
| | TGA | 1.0 wt % loss from 45-200° C., |
| | TMDSC | 280° C. (decomp. onset) |
| | | Reversing heat flow: |
| | | 61° C. (Tg, midpoint) |
| | | ΔCp 0.4 J/g · ° C. |
| | | Total heat flow: |
| | | 91° C. (exo, peak) |
| | | 178° C. (endo, onset) |

Solubility: Generally, the solubility of an amorphous form is higher than that of the corresponding crystal form, due to the lack of crystalline lattice forces in the amorphous state. Solubility of the amorphous form of Compound A was studied by slow addition of the amorphous form from an organic stock solution into the pH 6.5 phosphate-buffered saline (PBS) solution or 0.5% wt simulated intestinal fluid (SIF) in pH 6.5 PBS. When the amorphous solubility is reached, a drug-rich phase forms which typically scatters light (e.g., liquid-liquid phase separation or LLPS), which can be detected by scattering of UV/Visible light and/or by dynamic light scattering (DLS).

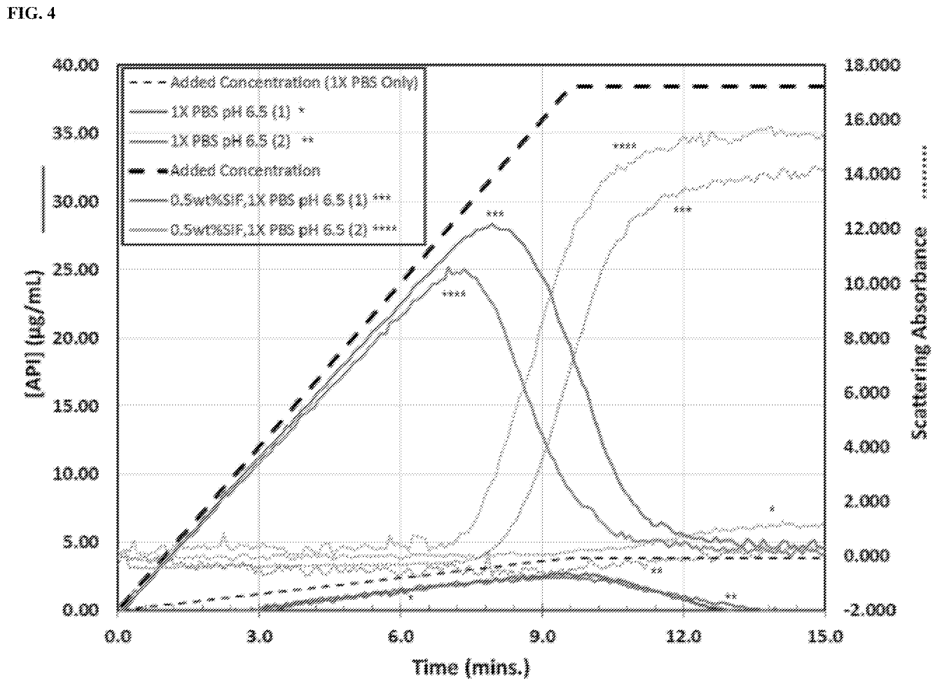

No scattering event prior to crystallization was observed. Based on the data obtained, the amorphous solubility in pH 6.5 PBS and 0.5% SIF (pH 6.5) was >2.5 μg/mL and >25 μg/mL, respectively (FIG. 4). While an exact concentration could not be determined in this test, the amorphous solubility appears to be at least 20× higher than the crystalline solubility in simulated intestinal media. FIG. 4 shows concentration (solids lines) and scattering (dotted lines) vs. time during addition of a 95:5 THF:Water solution of amorphous form of Compound A into blank PBS or 0.5% SIF in PBS.

### Example 4: Solid Dispersion Composition Study 1

Compound A has very low crystalline solubility and very high amorphous solubility enhancement. It crystallizes rapidly from supersaturated aqueous solutions, dosed alone or with pre-dissolved precipitation-inhibiting polymers. Amorphous form of Compound A has a moderate glass transition temperature (Tg=62° C.) and partially re-crystallizes during heating of the amorphous form (Class 2 glass former).

Based on these characteristics, compositions were prepared at 10% active loading with 5 different polymers or polymer blends (Table 4). All manufactured formulations were amorphous by x-ray powder diffraction (XRPD),

TABLE 4
Compositions

| SDD | | Yield | Potency |
|---|---|---|---|
| No. | Compositions | (%) | (mgA/g) |
| A | 10/90 Compound A/HPMCAS-H | 93 | 104 ± 0.1 |
| B | 10/80/10 Compound A/HPMCAS-H/ | 96 | 107 ± 0.1 |
| | Soluplus ® (graft copolymers of | | |
| | polyethyleneglycol, polyvinylcaprolactam, | | |
| | and polyvinylacetate) | | |
| C | 10/90 Compound A/HPMCAS-L | 94 | 102 ± 0.3 |
| D | 10/90 Compound A/PVP K30 | 91 | 106 ± 0.3 |
| E | 10/90 Compound A/Eudragit ® L100 | 95 | 102 ± 0.1 |
| | (methacrylic acid - methyl methacrylate | | |
| | co-polymer) | | |

mgA/g = milligrams of Compound A per gram of SDD composition

Five spray dried dispersion (SDD) compositions were successfully manufactured with high yields on a Bend Lab Dryer with 35 kg/hr drying capacity (BLD-35). All SDDs were sprayed at the same atomization pressure (120 psig), After spray drying, the SDDs were secondary dried in a heating vacuum tray dryer for about 24 hours to remove residual solvent. Manufacturing parameters are listed in Table 5.

TABLE 5
SDD composition manufacturing summary

| | |
|---|---|
| Batch size (g) | 3 |
| Solvent (w/w) | 9/1 DCM/methanol |
| Solids Content (wt %) | 4 |
| Atomizer | Schlick 2.0 |
| Drying Gas Flow Rate (g/min) | 500 |
| Solution Flow-rate (g/min) | ~35 |
| Atomization Pressure (psig) | 120 |
| Inlet Temperature (° C.) | 71 (for SDD-A); 77 |
| | (for SDD-B to SDD-E) |
| Outlet Temperature (° C.) | 35 |

All SDDs contained amorphous form of Compound A by X-ray diffraction analysis (FIG. 5). The Tg of each SDD was dominated by the type of polymer as determined by modulated differential scanning calorimetry (mDSC). A water or solvent loss peak is observed for each SDD, being most intense for SDD composition D (FIG. 6). The solid lines of the mDSC thermograms of FIG. 6 are the reverse heat flow and the dashed lines are the non-reversing heat flow. Summary of the mDSC data is listed in Table 6.

TABLE 6
Tabulated mDSC data for SDD compositions

| SDD No. | Tg (° C.) | ΔCp (J/(g° C.)) |
|---|---|---|
| A | 99 ± 0.5 | 0.33 ± 0.02 |
| B | 97 ± 0.6 | 0.33 ± 0.03 |
| C | 98 ± 0.2 | 0.34 ± 0.02 |
| D | 145 ± 0.03 | 0.29 ± 0.01 |
| E | 176 ± 2.0 | 0.41 ± 0.10 |

### Example 5: Dissolution and Suspension Stability of Solid Dispersion Compositions

Dissolution: The crystalline Compound A and the five SDD compositions (A-E from Example 4) is were tested in a 2-stage dissolution test designed to evaluate dissolution rate as well as inhibition of crystallization on transfer from simulated gastric to simulated intestinal media. Additionally, SDD composition E was also tested with additional HPMCAS-HF powder at a ratio of 1:1 SDD/HPMCAS-HF (SDD composition F).

For the gastric-to-intestinal buffer (G-IB) transfer dissolution test, pH 2 (“gastric”) media was added to each sample at a target dose concentration of 300 μgA/mL. After 30 minutes of exposure to gastric media, concentrated simulated intestinal buffer (TB) is added to reach a final composition of 0.5% SIF in PBS, pH 6.5 at half the dose concentration (150 μgA/mL). The concentration was monitored by U fiber optic probes in situ as well as being assessed by either microcentrifugation (15,800×g) or ultracentrifugation (380,000×g).

The microcentrifuge separates the precipitate (undissolved drug) and total solubilized drug Total solubilized drug consists of three solubilized species: freely solubilized drug (“free drug”), drug associated with bile salt micelles (“micelle bound drug”), and drug in small aggregate roughly 50-300 nm in size (“colloidal drug”). The ultracentrifuge further separates the colloidal drug and the supernatant contains only free drug and micelle bound drug. These three species have different activities in vivo and can be used to help differentiate formulations. Free drug is the smallest species and is the only species considered to partition directly into the cell membrane of the epithelium. Micelle bound drug can cross the unstirred mucous boundary layer and source free drug once a concentration gradient has been created by freely solubilized drug being absorbed. Finally, the drug-polymer colloids are a source of rapidly dissolving drug and may, in some cases, penetrate the unstirred mucous boundary layer.

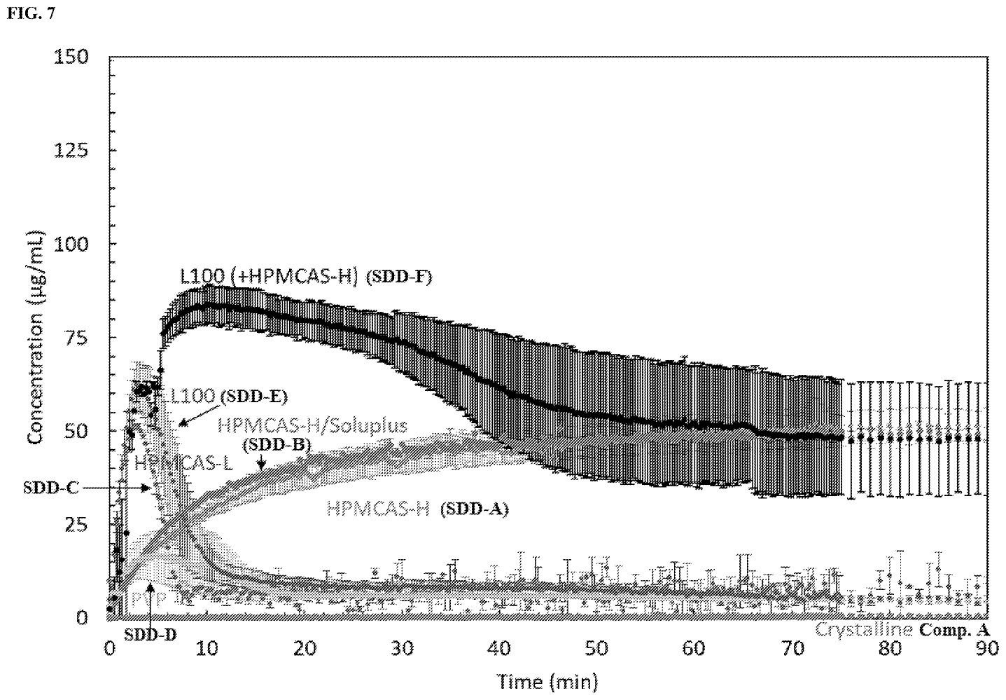

SDD compositions A and B dissolved slowly upon transfer from simulated gastric to simulated IB with no evidence of precipitation (see FIG. 7). SDD compositions C and E dissolved rapidly upon transfer to TB followed by rapid precipitation. SDD composition D reached a lower concentration than all the enteric SDDs and then precipitated. SDD composition F dissolved rapidly and mostly sustained throughout the dissolution test, possibly with some precipitation.

The concentration achieved and sustained in intestinal buffer was much higher than the crystalline Compound A for all SDD compositions but varied greatly between compositions. The highest concentration measured after ultracentrifugation (generally considered to be a measure of the actual dissolved drug concentration, or “free drug plus micellar bound drug”) was 33 μg/mL with SDD compositions A and B. The concentration measured by UV-Vis for SDD composition F was much higher, suggesting that some small colloidal species may be forming during dissolution of the composition, which are not indicative of the dissolved drug concentration. The concentration measured by HPLC after centrifugation was much lower for composition F and the final concentration of F and the HPMCAS-H based SDDs was similar, as determined after centrifugation or by in situ UV-Vis.

TABLE 7
Gastric to intestinal transfer dissolution results

| | Concentration (μgA/mL) | | | |
|---|---|---|---|---|
| | Gastric | IB | IB | IB |
| SDD No. | (5 min) | (5 min) | (90 min) | (90 min; ultra) |
| A | 6 | 28 | 35 | 33 |
| B | ND | 26 | 35 | 33 |
| C | ND | 11 | 5 | 4 |
| D | ND | 11 | 6 | 4 |
| E | ND | 12 | 8 | 5 |
| F | ND | 43 | 39 | 29 |
| Crystalline | ND | ND | 7 | ND |
| Compound A | | | | |

### Example 6: Solid Dispersion Composition Study 2

SDD compositions G-M were successfully manufactured with high yields on a Bend Lab Dryer with 35 kg/hr drying gas capacity (BLD-35) (Table 8). All SDDs were sprayed at the same atomization pressure (120 psig). After spray drying, the SDDs were secondary dried in a heating vacuum tray dryer at 40° C. for about 23 hours to remove residual solvent. Manufacturing parameters are listed in Table 9.

Secondary Drying was monitored by headspace gas chromatography in a separate tray drying space for an SDD composition of Table 8. Prior to secondary drying (wet sample), residual solvent in the SDD composition of Table 8 at storage temperature of 5° C. and 30° C. in sealed, stainless steel containers. Some solvent loss during storage and/or sampling was observed. SDD dried quickly during secondary drying, falling below ICH limits for residual DCM (600 ppm limit and permitted daily exposure of 6.0 mg/day) in less than 2 hours. Data supports secondary drying step of about 6 hours.

All manufactured formulations (stored at 2-8° C. after manufacturing) were amorphous by x-ray powder diffraction (XRPD), exhibiting the expected amorphous halo with no evidence of crystalline Compound A after manufacturing (FIG. 10). After 1 week at 50° C./75% RH, Compound A crystallized from SDD compositions L and M (FIG. 10),

TABLE 8
SDD Compositions

| SDD | | Yield | Potency |
|---|---|---|---|
| No. | Compositions | (%) | (mgA/g) |
| A¹ | 10/90 Compound A/HPMCAS-H | 93 | 104 ± 0.1 |
| G | 15/85 Compound A/HPMCAS-H | 95 | 157 |
| H | 20/80 Compound A/HPMCAS-H | 94 | 209 |
| I | 25/75 Compound A/HPMCAS-H | 96 | 261 |
| J | 30/70 Compound A/HPMCAS-H | 93 | 314 |
| K | 60/40 Compound A/Eudragit ® L100 | 91 | 616 |
| | (methacrylic acid - methyl methacrylate | | |
| | co-polymer) | | |
| L | 20/70/10 Compound A/PVP-VA64/Soluplus ® | 91 | 215 |
| | (graft copolymers of polyethyleneglycol, | | |
| | polyvinylcaprolactam, and polyvinylacetate) | | |
| M | 20/80 Compound A/Soluplus ® (graft | 101 | 210 |
| | copolymers of polyethyleneglycol, | | |
| | polyvinylcaprolactam, and polyvinylacetate) | | |

¹From Example 4. mgA/g = milligrams of Compound A per gram of SDD composition

TABLE 9
SDD composition manufacturing summary

| Batch size (g) | 5-10 g |
|---|---|
| Solvent (w/w) | 9/1 DCM/methanol |
| Solids Content (wt %) | 4 |
| Atomizer | Schlick 2.0 |
| Drying Gas Flow Rate (g/min) | 500 |
| Solution Flow-rate (g/min) | ~35 |
| Atomization Pressure (psig) | 120 |
| Inlet Temperature (° C.) | 90 (SDD-G); 85 (SDD-H and |
| | SDD-I); 84 (SDD-J); 76 |
| | (SDD-K); 75 (SDD-L and SDD-M) |
| Outlet Temperature (° C.) | 35 |

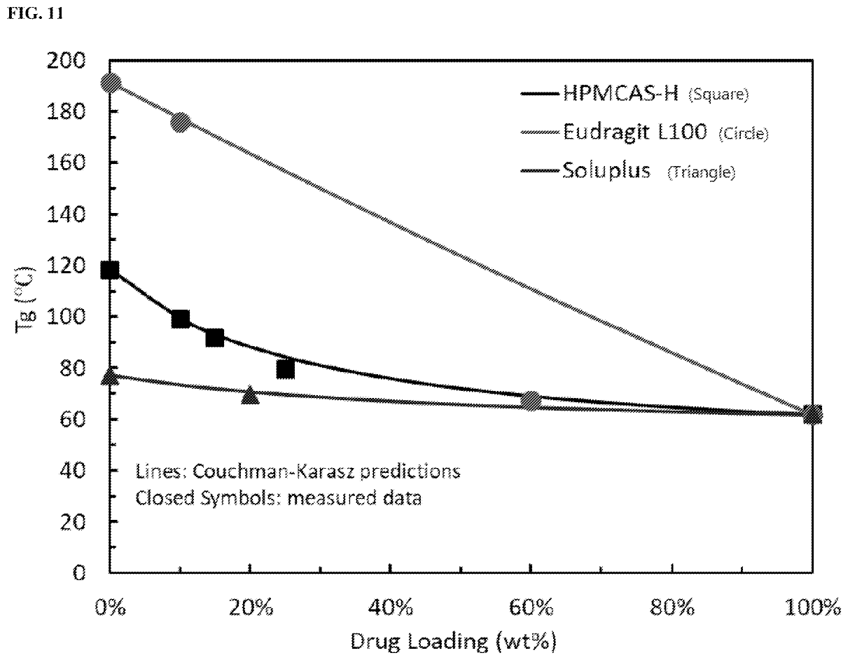

Tg of selected SDD compositions were determined by modulated differential scanning calorimetry (mDSC). The dry Tg (Tg determined under dry conditions) decreased with increased loading of Compound A (FIG. 11). In FIG. 11, the lines represent the predicted values and the closed symbols represent measured data. The dry Tg of all the SDD compositions showed sufficiently high for storage under dry conditions Table 10. The Tg decreased at elevated RH due to plasticization by absorbed water. SDD compositions 1 and K absorbed about 3% water at 75% RH while SDD composition M absorbed about 6% water, which is consistent with the decrease in Tg at 75% RH observed for SDD composition M.

TABLE 10
Tabulated mDSC data for SDD compositions

| | Dry Tg | 75% RH Tg | Water at 75% |
|---|---|---|---|
| SDD No. | (° C.) | (° C.) | RH (wt %) |
| A | 101.2 | — | — |
| G | 91.6 | — | — |
| H | 89 | — | — |
| I | 79.4 | 60.3 | 3 |
| K | 67.5 | 58.4 | 3 |
| M | 69.8 | 28.9 | 6 |

Dissolution was measured by gastric-to-intestinal buffer (G-IB) transfer dissolution test according to Example 5 for SDD compositions G-M dissolved to concentrations at or above the amorphous solubility and maintained supersaturation for the duration of the test (90 min in simulated intestinal fluid). Precipitation to a lower concentration was observed for SDD composition I and at least one replicate of SDD composition K with external HPMCAS-H (Table 11).

TABLE 11
Gastric to intestinal transfer dissolution results

Ultracentrifuge Concentration
(μgA/mL)

| SDD No. | 5 min | 90 min |
|---|---|---|
| G | 36 | 62 |
| H | 27 | 61 |
| I | 26 | 55 |
| J | 32 | 37 |
| K with external | (29, 36) | (38, 28) |
| HPMCAS-H | | |
| L | 32 | 41 |
| M | 34 | 52 |

Determined by HPLC after centrifugation at 380,000 × g (ultra)

Suspension stability was determined according to Example 5 for suspensions prepared with SDD compositions H, I, and M in 0.5% Methocel A4M (aqueous). Suspensions appeared to be syringeable. All three suspensions were stable for at least 20 h.

Particle size distribution of SDD composition with HPMCAS-H from Table 8 was measured via laser light scattering (Table 12). A Malvern Mastersizer 3000 was used with an Aero dry dispersion sample cell to perform the measurement. Additionally, a pressure titration curve was performed where the dispersive air pressure (pressure used to disperse sample for analysis) was varied. In general, low dispersive air pressures can lead to incomplete dispersion of the sample and high pressures can lead to breakage of primary particles.

TABLE 12
Particle Size Distribution of an SDD composition
with HPMCAS-H at 3 barg dispersive pressure

| D(v 0.1) | D(v 0.5) | D(v 0.9) | D[3,2] | D[4,3] | |
|---|---|---|---|---|---|
| μm | μm | μm | μm | μm | Span |
| 16.9 | 55.4 | 109.3 | 22.0 | 60.3 | 1.7 |

Bulk and tapped density were measured for an SDD composition with HPMCAS-H (Table 13). The Carr Index is based on the ratio of tapped and bulk density, where lower values indicate higher flowability. A Carr index of 37 is considered “poor” flowing by literature ranges and is the reason dry granulation of SDDs is performed prior to tablet compression. A Carr index of 37 suggests sufficient flowability to enable pre-granulation blending and feed into the roller-compactor. Water content of the SDD composition HPMCAS-H was analyzed via Coulometric Karl Fischer and was found to contain 1.0±0.1 wt % water.

TABLE 13
Bulk and Tapped Density of SDD composition HPMCAS-H

| | | | Bulk | Tapped | | |
|---|---|---|---|---|---|---|
| | Bulk | Tapped | Specific | Specific | Carr | |
| | Density | Density | Volume | Volume | Index | Hausner |
| Value | (g/mL) | (g/mL) | (mL/g) | (mL/g) | (%) | Ratio |
| Mean | 0.21 | 0.34 | 4.7 | 2.9 | 37 | 1.60 |
| Min-Max | 0.21-0.22 | 0.34-0.35 | 4.6-4.7 | 2.9-2.9 | 37-38 | 1.59-1.6 |

### Example 7 Solid Dispersion Composition Study 3

SDD compositions H-J and N-R were successfully manufactured with high yields on a Bend Lab Dryer with 35 kg/hr drying gas capacity (BLD-35) (Table 14). All SDDs were sprayed at the same atomization pressure (120 psig). After spray drying, the SDDs were secondary dried in a heating vacuum tray dryer at 40° C. with 3 liters per minute or 2.5 liters per minute of N₂ sweep gas for about 18.5-23 hours to remove residual solvent. Manufacturing parameters are listed in Table 15.

TABLE 14
SDD Compositions

| | | | | Residual | Residual |
|---|---|---|---|---|---|
| SDD | | Yield | Potency | MeOH | DCM |
| No. | Compositions | (%) | (mgA/g) | (wt %) | (wt %) |
| H | 20/80 Compound A/HPMCAS-H | 82 | 210 | ND | ND |
| I | 25/75 Compound A/HPMCAS-H | 85 | 260 | ND | ND |
| J | 30/70 Compound A/HPMCAS-H | 96 | 310 | ND | ND |
| N | 35/65 Compound A/HPMCAS-H | 95 | 360 | ND | ND |
| O | 40/60 Compound A/HPMCAS-H | 96 | 410 | ND | 0.01 |
| P | 45/55 Compound A/HPMCAS-H | 93 | 450 | <LOQ | 0.03 |
| Q | 50/50 Compound A/HPMCAS-H | 93 | 500 | <LOQ | 0.06 |
| R | 75/25 Compound A/HPMCAS-H | 93 | 750 | <LOQ | 0.09 |

mgA/g = milligrams of Compound A per gram of SDD composition;
LOQ = limit of quantification

TABLE 15
SDD composition manufacturing summary

| Batch size (g) | 1-15 g |
|---|---|
| Solvent (w/w) | 9/1 DCM/methanol |
| Solids Content (wt %) | 4 |
| Atomizer | Schlick 2.0 |
| Drying Gas Flow Rate (g/min) | 500 |
| Solution Feed Rate (g/min) | 30-44 |
| Atomization Pressure (psig) | 120 |
| Inlet Temperature (° C.) | 92 (SDD-H and SDD-N); 100 (SDD-I); |
| | 97 (SDD-J); 89 (SDD-O); 85 (SDD-P, |
| | SDD-Q, and SDD-R) |
| Outlet Temperature (° C.) | 40-42 |

All manufactured formulations (stored at 2-8° C. after manufacturing) were amorphous by x-ray powder diffraction (XRPD), exhibiting the expected amorphous halo with no evidence of crystalline Compound A after manufacturing (FIG. 12).

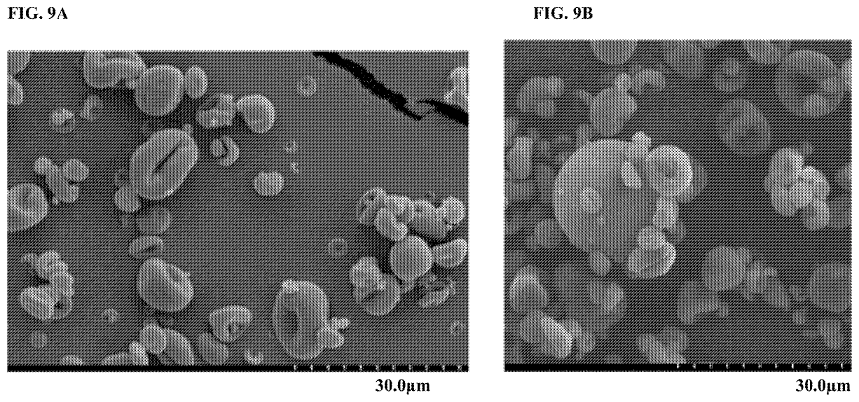

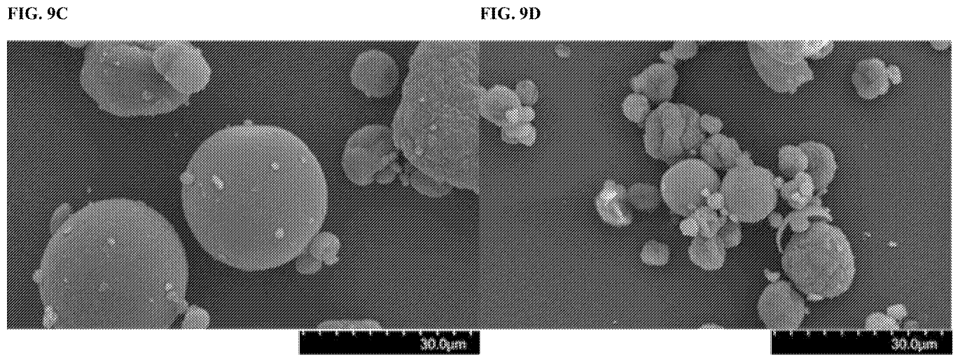

SEM: Scanning electron microscopy (SEM) imaging of the SDD compositions of Table 14 showed standard particle morphology of shrunken and collapsed spheres (FIGS. 8A-8D and 9A-9D). No signs of crystalline material or particle fusing were present. Particles of SDD composition Q and R were visibly rougher. This is typical of spray dried materials with a high content of low molecular weight material as skin that forms on drying is typically much less elastic than with high polymer content.

mDSC: Tg of SDD compositions (Table 17) were determined by modulated differential scanning calorimetry (mDSC) using double scan method according to Table 16. The Tg at elevated RH was depressed relative to the dry Tg as expected with increased water uptake (Table 18). The Tg depression was greatest for SDD composition with the lowest active concentration because amorphous Compound A is less hygroscopic than HPMCAS-H. As a result, at elevated RH there was a minimum in the Tg vs loading curve at about 35% Compound A.

TABLE 16
Double Scan mDSC Method Parameters

| | |
|---|---|
| Cycle 1 | |
| Temperature Range (° C.) | 0 to 120 |
| Scan Rate (° C./min) | 2.5 |
| Modulation (° C./min) | ±1.5 |
| Ramp 1 (° C.) | 0 to 60 |
| Isothermal Hold (min) | 40 (1 month samples only) |
| Ramp 2 (° C.) | 60 to 120 |
| Cycle 2 | |
| Temperature Range (° C.) | 0 to 200 |
| Scan Rate (° C./min) | 2.5 |
| Modulation (° C./min) | ±1.5 |
| Sample Preparation | 4-6 mg loose powder |
| Pan Type | Tzero Aluminum Standard Pan at <5% RH, |
| | hermetic pan at higher RH |
| T_{g} determination | Midpoint by half height |

TABLE 17
Tabulated mDSC data for SDD compositions

| | T_{g} | ΔCₚ | T_{C Onset} | T_{M Onset} | ΔHₘ |
|---|---|---|---|---|---|
| SDD No. | (° C.) | (J/g · K) | (° C.) | (° C.) | (J/g)* |
| H | 81.8 | 0.35 | ND | ND | ND |
| I | 78.0 | 0.32 | ND | ND | ND |
| J | 74.8 | 0.34 | ND | ND | ND |
| N | 72.6 | 0.34 | 146.2 | 174.8 | 2.9 |
| O | 70.8 | 0.35 | 143.0 | 174.5 | 12.8 |
| P | 68.2 | 0.32 | 114 | 178 | 38 |
| Q | 69.1 | 0.36 | 123 | 173 | 71 |
| R | 64.8 | 0.15 | 97 | 183 | 70 |

*ΔHₘ is approximate due to overlap of crystallization and melt transitions

TABLE 18
Tg vs. RH

| | T_{g} (° C.) | | |
|---|---|---|---|
| SDD No. | 0% RH | 50% RH | 75% RH |
| I | 78.5 | — | 60.3 |
| N | 72.6 | 58.9 | 48.1 |
| O | 70.8 | — | 50.1 |
| Q | 69.1 | 59.2 | 51.6 |
| R | 64.8 | 60.5 | 55.9 |

Dissolution: A non-sink dissolution test was performed in scintillation vials. The SDD compositions were dosed at 0.150 mgA/mL in 0.5 wt % SIF, 1×PBS, pH 6.5. Manual pulls were, taken and ultra-centrifuged to evaluate the extent of dissolution and sustainment of supersaturation (FIG. 13). Concentration of dissolved species was monitored using Pion UV-Vis probes and manual pulls that were ultra-centrifuged at 360,000×g and analyzed via HPLC. Dissolution rate and the time to onset of precipitation was determined using manual ultra-pull values and appeared to decrease with increased drug loading. The test showed comparable onsets of precipitation for all four high-loaded SDDs. A decrease in concentration is measured over time for all formulations, with more rapid and/or earlier decrease in concentration for SDDs with higher active loading. There is a gradual change in performance between 20 and 50% active loading. The 75% active formulation dissolves very slowly and/or precipitated by the first time point (30 minutes).

The impact of external polymer on the SDD compositions J, N, and O was tested via non-sink dissolution in 20 mL scintillation vials (FIG. 14). External polymer was added as a powder to bring the ratio of Compound A/Polymer to 27/73. The SDD compositions were dosed at 0.150 mgA/mL in 0.5 wt % SIF, 1×PBS, pH 6.5. Manual pulls were taken and ultra-centrifuged to evaluate the extent of dissolution and sustainment of supersaturation in the presence of external polymer. The external HPMCAS-H appeared to show the greatest delay in precipitation for SDD compositions N and O.

Water Content: The SDD compositions showed comparable water content (Karl Fisher (KF) analysis) on stability (Table 19). The water content measured at 2 months was lower than that measured at 1 month. All samples were similar to each other within a test set. The variability in water content between the two stability pulls is most likely due to the relative humidity in the laboratory during the analyses and is not representative of a “loss” of water on stability.

TABLE 19
Water Content

| | | wt % H₂O (Range) | |
|---|---|---|---|
| SDD No. | Stability Condition | 1 mo. | 2 mo. |
| I | 25° C./60% RH Closed | 0.83 (0.01) | 0.23 (0.01) |
| | 40° C./75% RH Closed | 0.7 (0.19) | 0.41 (0.01) |
| J | 25° C./60% RH Closed | 0.71 (0.02) | 0.18 (0.01) |
| | 40° C./75% RH Closed | 0.63 (0.07) | 0.36 (0.02) |
| N | 25° C./60% RH Closed | 0.76 (0.01) | 0.17 (0.01) |
| | 40° C./75% RH Closed | 0.69 (0.03) | 0.33 (0.00) |
| O | 25° C./60% RH Closed | 0.62 (0.00) | 0.19 (0.01) |
| | 40° C./75% RH Closed | 0.59 (0.01) | 0.34 (0.01) |

Particle size and Density: Particle size distribution of an SDD composition with HPMCAS-H from Table 14 was measured via laser light scattering and bulk and tapped density (Table 20).

TABLE 20
Particle Size Distribution and Density of an SDD Composition

| | | | | | | Bulk | Tap |
|---|---|---|---|---|---|---|---|
| D(v 0.1) | D(v 0.5) | D(v 0.9) | D[3,2] | D[4,3] | | Density | Density |
| μm | μm | μm | μm | μm | Span | (g/mL) | (g/mL) |
| 12.4 | 43.8 | 87.9 | 15.5 | 47.9 | 1.7 | 0.29 | 0.42 |

### Example 8: 50 mg and 100 mg Tablets

Initial screening of tablet excipients was performed by manufacturing compacts of the solid dispersion composition of Compound A with a generic blend (Table 21), as well as excipients that may present chemical stability issues. The compacts were stored at 50° C./75% RH (open dish) for 7 days and then analyzed via HPLC. Compared to the solid dispersion of Compound A only compact there was little increase in total impurities in any of the samples.

TABLE 21
Screened Excipients

| Function | Material | Percentage |
|---|---|---|
| Ductile Filler | Microcrystalline cellulose | 56.7 |
| Brittle Filler | Lactose monohydrate | 28.3 |
| Disintegrant | Croscarmellose sodium | 12.0 |
| Glidant | Colloidal silicon dioxide | 1.0 |
| Lubricant | Sodium stearyl fumarate | 2.0 |

Three formulation were chosen for this study as shown in Table 22. All formulations included 1-10% intragranular and 1-0% extragranular disintegrant. Formulations A and C can be directly compared to assess the impact of solid dispersion composition loading on tablet performance and Formulations B and C can be directly compared to assess the impact of disintegrant selection. Tablets were prepared at bench scale using slugging to simulate roller compaction. A common blend of each formulation was prepared and used to compress both 50 mg and 100 mg target dose tablets. Table 23 and Table 24 summarize the compression and disintegration performance of each formulation. Disintegration tests were performed in a USP<701> style basket rack assembly using 0.01N HCl at ˜37° C. as the disintegration media.

TABLE 22
Tablet Formulations

| Formulation ID | A | B | C |
|---|---|---|---|
| Tablet Weight (mg) | 420 | 360 | 360 |
| Tablet Dose Target (mg) | 50.0 | 50.0 | 50.0 |
| Tablet Dose/Weight (mg/mg) | 50/420 | 50/360 | 50/360 |

| | | |
|---|---|---|
| Function | Ingredient | % of Blend |

Intragranular

| | | | | |
|---|---|---|---|---|
| Active | 15-45% Compound A Solid Dispersion | 55-64% | 65-75% | 65-75% |
| | of with HPMCAS-H | | | |
| Filler | Microcrystalline cellulose | 10-20% | 5-15% | 5-15% |
| Filler | Lactose Monohydrate | 10-20% | 5-15% | 5-15% |
| Disintegrant | Croscarmellose sodium | 1-10% | 0% | 1-10% |
| Disintegrant | Crospovidone | 0% | 1-10% | 0% |
| Glidant | Silicon dioxide | 0.1-1.5% | 0.1-1.5% | 0.1-1.5% |
| Lubricant | Magnesium stearate | 0.1-1.5% | 0.1-1.5% | 0.1-1.5% |

Extragranular

| | | | | |
|---|---|---|---|---|
| Disintegrant | Croscarmellose sodium | 1-10% | 0% | 1-10% |
| Disintegrant | Crospovidone | 0% | 1-10% | 0% |
| Glidant | Silicon dioxide (Syloid ® 244 FP) | 0.1-1.5% | 0.1-1.5% | 0.1-1.5% |
| Lubricant | Magnesium stearate | 0.1-1.5% | 0.1-1.5% | 0.1-1.5% |
| % Polymer | | 40-55% | 50-65% | 50-65% |

TABLE 23
Tablet Compression Characteristics
and Disintegration - 50 mg Tablets

| Parameter | Tablet A | Tablet B | Tablet C |
|---|---|---|---|
| Tooling Size (inch) | 0.2839 × 0.5879 Oval | | |
| Avg. Tablet Weight (mg) | 419.9 | 360.8 | 360.8 |
| Avg. Tablet Thickness (mm) | 6.14 | 5.66 | 5.56 |
| Avg. Tablet Hardness (kP) | 18.3 | 16.4 | 17.8 |
| Avg. Tablet Tensile Strength (MPa) | 1.91 | 1.96 | 2.10 |
| Avg. Compression Force (kN) | 10.0 | 10.5 | 10.3 |
| Avg. Compression Stress (MPa) | 123 | 130 | 113 |
| Avg. Tablet Solid Fraction¹ | 0.77 | 0.73 | 0.65 |
| Disintegration Time, n = 2 (min:sec) | 0:31 | 0:23 | 0:33 |
| | 0:33 | 0:29 | 0:33 |

¹Assumed true density = 1.4000 g/mL.

TABLE 24
Tablet Compression Characteristics
and Disintegration - 100 mg Tablets

| Parameter | Tablet A | Tablet B | Tablet C |
|---|---|---|---|
| Tooling Size (inch) | 0.4062 × | 0.3577 × | |
| | 0.7500 Oval | 0.7154 Oval | |
| Avg. Tablet Weight (mg) | 841.5 | 719.9 | 719.2 |
| Avg. Tablet Thickness (mm) | 6.39 | 7.16 | 7.01 |
| Avg. Tablet Hardness (kP) | 25.0 | 25.5 | 27.1 |
| Avg. Tablet Tensile Strength | 2.07 | 1.91 | 2.10 |
| (MPa) | | | |
| Avg. Compression Force (kN) | 20.0 | 14.3 | 14.9 |
| Avg. Compression Stress (MPa) | 127 | 111 | 115 |
| Avg. Tablet Solid Fraction¹ | 0.75 | 0.73 | 0.74 |
| Disintegration Time, n = 2 | 1:09 | 0:49 | 1:45 |
| (min:sec) | 1:43 | 1:09 | 2:21² |

¹Assumed true density = 1.4000 g/mL;
²Disintegration float was observed to be sticking. Tablet appeared to be disintegrated by around 1:30 (min:sec)

Tablet B (50 mg and 100 mg) was selected for further testing, thus, prepared in bulk. All intragranular materials except for the lubricant was blended, then de-lumped. Lubricant was de-lumped then added to the remainder of the intragranular materials and blended. Roller compaction was carried out on the intragranular blend. To that, extragranular disintegrant and glidant was added and blended. Extragranular lubricant was delumped and added to the blend. For 50 mg tablet, tablet was compressed using tooling available for the rotary press (tooling size ⅜″ SRC) to achieve a target mass of 360 mg, target tensile strength of 2.0 MPa, and target tablet hardness of 16.6 kP. For 50 mg tablet, tablet was compressed using tooling available for the rotary press (tooling size 0.3577″×0.7154″) to achieve a target mass of 720 mg, target tensile strength of 2.0 MPa, and target tablet hardness of 25.0 kP. Both tablet batches were dedusted after compression.

Tablet Friability: Friability was tested using a USP<1216> style drum device. Friability was measured on the 1.5 and 2.5 MPa range and from two time points of the 2.0 MPa tablets for the 50 mg Tablet B as well as 100 mg Tablet B (Table 25). The friability for these tablets was well below the USP<1216> guidance of not more than 1.0% weight loss at 100 drops. No gross damage to the tablets was observed at any of the drop counts.

TABLE 25
Tablet B Friability Results

| Tablet Size | Tensile Strength | 100 drops | 300 drops | 500 drops |
|---|---|---|---|---|
| 50 mg | 1.5 MPa | 0.1% | N/A | N/A |
| 50 mg | 2.5 MPa | 0.0% | 0.1% | 0.2% |
| 50 mg | 2.0 MPa | 0.1% | 0.2% | 0.3% |
| | (0-10 min) | | | |
| 50 mg | 2.0 MPa | 0.0% | 0.2% | 0.3% |
| | (20-30 min) | | | |
| 100 mg | 2.0 MPa | 0.1% | 0.3% | 0.5% |

Tablet Disintegration: All disintegration tests were performed in a USP<701> style basket rack assembly using 0.01N HCl at ca. 37° C. as the disintegration media (Table 26). Disintegration times of the 2.0 MPa tablets are similar upon transfer from bench scale to rotary press manufacture. Disintegration times remained acceptable when increasing tablet tensile strength to 2.5 MPa, maintaining a disintegration time of less than 300 seconds (5 minutes).

TABLE 26
Tablet B Disintegration (seconds)

| Tablet Tensile Strength (MPa) | 50 mg | 100 mg |
|---|---|---|
| and Prep Scale | Tablet B | Tablet B |
| 2.0 bench scale | 26 | 59 |
| 2.0 rotary press | 39 | 42 |
| 1.5 rotary press | 23 | N/A |
| 2.5 rotary press | 247 | N/A |

### Example 9: 200 mg Tablets

200 mg strength tablets (Table 27) were prepared according to Example 8. The tablets were further film coated (Opadry® QX) to a target coat weight gain of 1-5% weight gain in a pan coater. Summary of the film coated 200 mg tablets are shown in Table 28.

TABLE 27
200 mg Tablet B

| Formulation ID | D |
|---|---|
| Tablet Dose/Weight (mg/mg) | 200/825 |

| | | |
|---|---|---|
| Function | Ingredient | % of Blend |

Intragranular

| | | |
|---|---|---|
| Active | 15-45% Compound A Solid Dispersion | 65-75% |
| | of with HPMCAS-H | |
| Filler | Microcrystalline cellulose | 5-15% |
| Filler | Lactose Monohydrate | 5-15% |
| Disintegrant | Crospovidone | 1-10% |
| Glidant | Silicon dioxide | 0.1-1.5% |
| Lubricant | Magnesium stearate | 0.1-1.5% |

Extragranular

| | | |
|---|---|---|
| Disintegrant | Crospovidone | 1-10% |
| Glidant | Silicon dioxide (Syloid ® 244 FP) | 0.1-1.5% |
| Lubricant | Magnesium stearate | 0.1-1.5% |

| | |
|---|---|
| % Polymer | 40-55% |

TABLE 28
Tablet Compression Characteristics and Disintegration

| Parameter | Tablet D |
|---|---|
| Tooling Size (inch) | 0.4062 × 0.7500 Oval |
| Avg. Tablet Core Weight (mg)² | 825.1 |
| Avg. Tablet Core Thickness (mm)² | 6.22 |
| Avg. Tablet Hardness (kP)² | 24.5 |
| Avg. Tablet Tensile Strength (MPa)² | 2.03 |
| Avg. Compression Force (kN) | 27.0 |
| Avg. Compression Stress (MPa) | 173 |
| Avg. Tablet Solid Fraction¹ | 0.85 |
| Avg. Coating Wet Coat Weight Gain | 2.8 |
| (wt %)³ | |
| Avg. Coated Tablet Weight (mg) | 850.6 |
| Tablet Core Friability (%) [100, | 0.06, 0.16, 0.23 |
| 300, 500 drops] | |
| Avg. Disintegration Time | Tablet Core = 2:38 (n = 6) |
| (min:sec) | Coated Tablet = 2:36 (n = 2) |

¹True density = 1.3444 g/mL;
²n = 30 tablets total;
³Coat weight gain (wt %) calculated based on warmed tablet core weight (827.1 mg).

### Biological Assays

### Example 10: Pharmacokinetics (PK) Study of Compound A in Animals

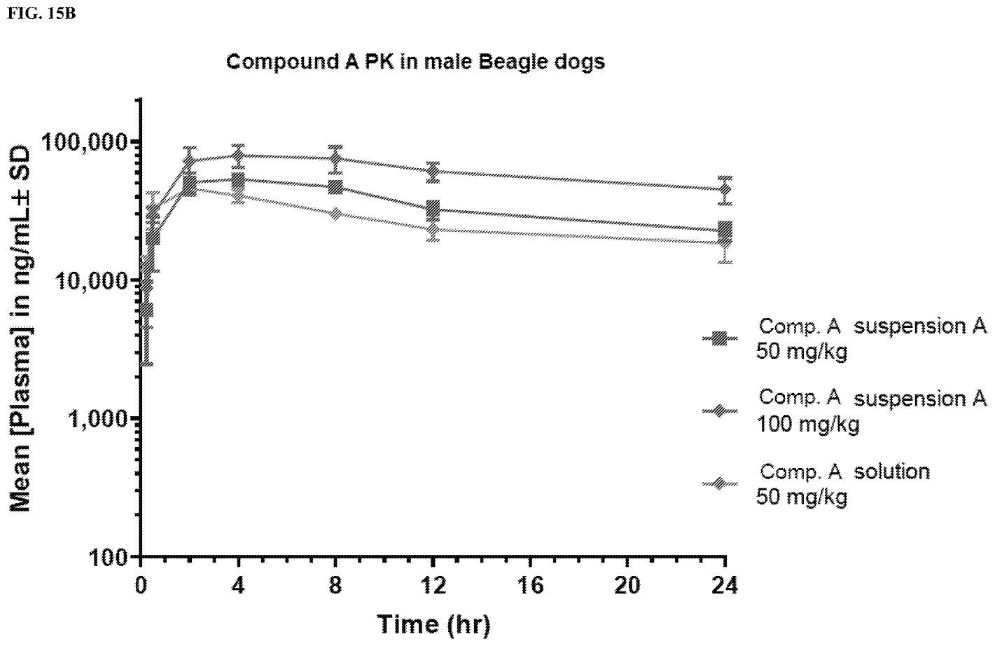

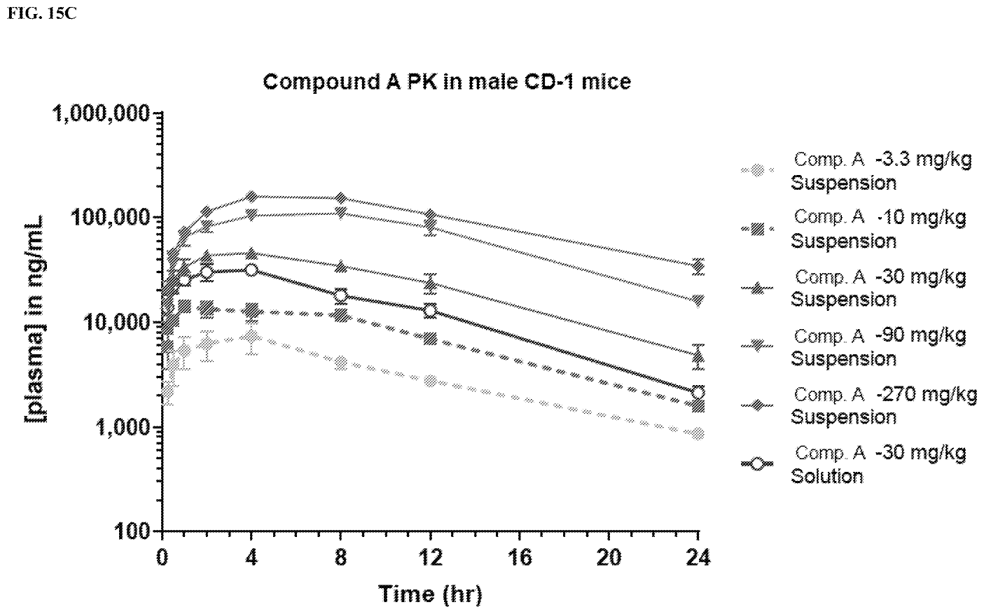

A single PO dose of Compound A in three different formulations were administered to male Sprague-Dawley (SD) rats. Animals in Group 1 (Solution) received Compound A by oral gavage (PO) administration at 30 mg/kg, which was formulated in 4% NMP, 20% Capmul MCM, 21% Vitamin E TPGS, 55% PEG400. Animals in Group 2 (Suspension A) received Compound A by oral gavage (PO) administration at 100 mg/kg, which was formulated in SDD formulation with HPMCAS-H in 0.5% Methocel A4M in water. Mean plasma of Compound A was measured over 24-hour period after administration of Compound A (FIG. 15A) and PK parameters were determined (Table 29).

TABLE 29
PK parameters of Compound A in male SD rats

| | | Dose | T½ | Tₘₐₓ | Cₘₐₓ | AUC₀₋ₗₐₛₜ |
|---|---|---|---|---|---|---|
| Group | Formulation | (mg/kg) | (h) | (h) | (ng/mL) | (ng · h/mL) |
| 1 | Solution | 30 | 7.6 | 9.3 | 26,800 | 399,000 |
| 2 | Suspension A | 100 | 6.2 | 5.3 | 89,800 | 1,180,000 |

A single PO dose of Compound A in two different formulations were administered to male Beagle dogs. Mean plasma of Compound A was measured over 24-hour period after administration of Compound A (FIG. 15B) and PK parameters were determined (Table 30).

TABLE 30
PK parameters of Compound A in male Beagle dogs

| | Dose | T½ | Tₘₐₓ | Cₘₐₓ | AUC₀₋ₗₐₛₜ |
|---|---|---|---|---|---|
| Formulation | (mg/kg) | (h) | (h) | (ng/mL) | (ng · h/mL) |
| Solution¹ | 50 | 21.9 | 2 | 45,800 | 648,000 |
| Suspension A² | 50 | 16.2 | 3.3 | 55,500 | 845,000 |
| Suspension A² | 100 | 24 | 5.3 | 80,600 | 1,450,000 |

¹Crystalline Compound A dissolved in 5% NMP, 20% vitamin E TPGS, 20% PEG 300, 0.5% SLS, 1.09% HPMCAS-H, and 53.91% water.
²Same as Suspension A in Table 29.

A single PO dose of Compound A in different doses in solid dispersion and at 30 mg/kg dose of Compound A in solution were administered to male CD-1 mice. Mean plasma of Compound A was measured over 24 hour period after administration of Compound A (FIG. 15C) and PK parameters were determined (Table 31). Data demonstrates high exposure of Compound A is possible when Compound A is formulated as a solid dispersion.

TABLE 31
PK parameters of Compound A in male CD-1 mice

| | Dose | AUC₀₋ₗₐₛₜ | AUC_{0-inf} | Cₘₐₓ |
|---|---|---|---|---|
| Formulation | (mg/kg) | (ng · h/mL) | (ng · h/mL) | (ng/mL) |
| Solution¹ | 30 | 336,000 | 352,000 | 32,033 |
| Suspension A² | 3.3 | 78,100 | 86,900 | 7,363 |
| Suspension A² | 10 | 177,000 | 189,000 | 14,300 |
| Suspension A² | 30 | 565,000 | 604,000 | 45,900 |
| Suspension A² | 90 | 1,570,000 | 1,700,000 | 109,667 |
| Suspension A² | 270 | 2,310,000 | 2,690,000 | 162,000 |

¹Crystalline Compound A dissolved in 9% DMSO, 9% NMP, 27% solutol, and 55% PEG400.
²Same as Suspension A in Table 29.

### Example 11: In Vivo Activity of Solid Dispersion of Compound a in Xenograft Models

Compound A's activity was measured in a variety of castrate-sensitive and castrate-resistant prostate cancer (CRPC) xenograft models (Table 32).

For example, intact male nude mice were engrafted with HID28 tumor fragments and once tumors reached ˜150 mm³ the mice were castrated. The mice were enrolled in the experiment once the tumors started to regrow, 10 days post-castration. Compound A was tested in the HID28 PDX model at 60 mg/kg, QD for 28 days using SDD formulation with HPMCAS-H in 0.5% Methocel A4M in water.

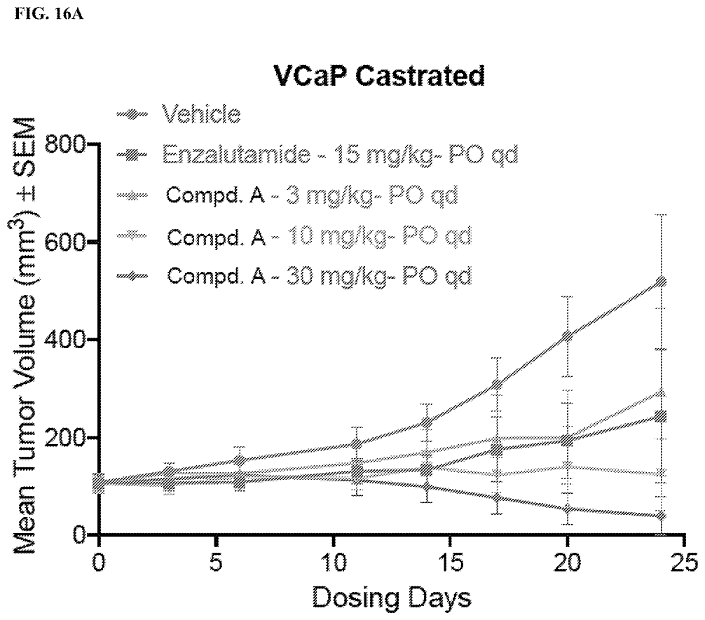

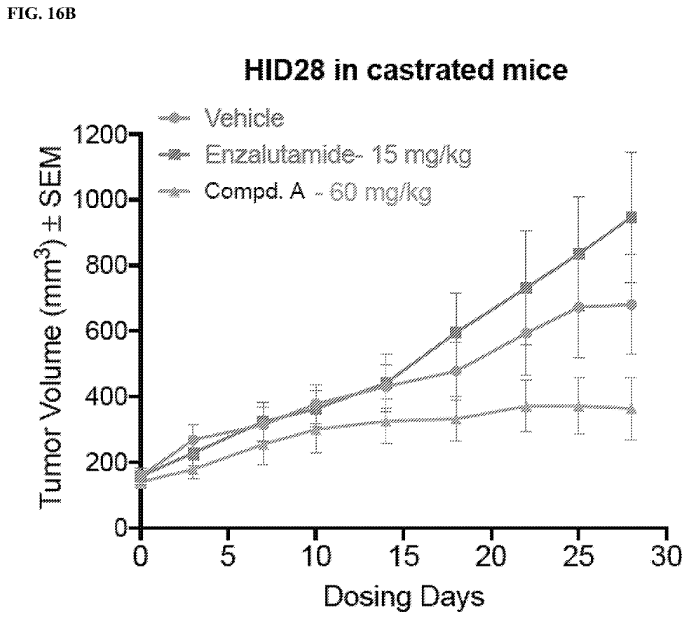

Example of anti-tumor activity in castrated SCID Beige male mice bearing VCaP tumors (n=5-6) are shown in FIG. 16A. Mean tumor volume in castrated nude mice bearing enzalutamide resistant patient-derived xenograft (PDX) model HID28 was measured after oral treatment with solid dispersion composition of Compound A or enzalutamide for 28 days (n=6-7 per group) is shown in FIG. 16B.

TABLE 32
Summary of in vivo activity of Compound A in a subset of prostate cancer
xenograft models bearing various AR-dependent and AR-independent tumors

| in vivo | | Male Mice | | Dose | | | TGI | |
|---|---|---|---|---|---|---|---|---|
| model | AR status | Condition | Compound | (mg/kg) | Formulation | Regimen | (%) | p value |
| LNCaP | AR FL | Castrated | Enzalutamide | 30 | Solution | qd24 | >100% | p < 0.0001 |
| | | | Compound A | 60 | | | 93% | p < 0.0001 |
| VCaP | Amplified AR | Castrated | Enzalutamide | 15 | Solution | qd24 | 66% | p < 0.05 |
| | Some AR-V7 | | Compound A | 3 | | | 54% | p < 0.01 |
| | | | Compound A | 10 | | | 95% | p < 0.0001 |
| | | | Compound A | 30 | | | >100% | p < 0.0001 |
| LNCaP95 | High level AR-V7 | Castrated | Enzalutamide | 15 | Solution | qd24 | <0% | NS |
| | Other oncogenic | | Compound A | 30 | | | 54% | p < 0.0001 |
| | pathways | | | | | | | |
| 22Rv1 | High level AR-V7 | Castrated | Compound A | 30 | Solution | qd28 | 48% | p < 0.0001 |
| | Other oncogenic | | | | | | | |
| | pathways | | | | | | | |
| HID28 | AR + PSA + | Castrated | Enzalutamide | 15 | Solution | qd28 | <0% | NS |
| | AR-V7 unknown | | Compound A | 60 | SDD | | 58% | p < 0.05 |
| PC-3 | Non functional AR | Intact | Enzalutamide | 15 | Solution | qd28 | 0% | NS |
| | Other oncogenic | | Compound A | 30 | | | 0% | NS |
| | pathways | | | | | | | |

Statistics: 2-way ANOVA with Dunnett correction for multiple comparisons.
NS: Non significant.
TGI: Tumor Growth Inhibition

### Example 12. Compound A in Patients with Metastatic Castration-Resistant Prostate Cancer

Phase I clinical research study of Compound A is studied as a treatment for patients with prostate cancer. All patients in the study receives Compound A at a once daily oral dose of 200 mg, 400 mg, 600 mg, 800 mg, or 1000 mg. Compound A in this study is provided as an SDD composition with HPMCAS-H. One cycle of treatment is 28 days of dosing.

The primary safety variable for Part 1a of the study is the incidence of protocol-defined dose limiting toxicity (DLT) during the DLT assessment period (first 28 days of dosing). The DLTs will be characterized by type, frequency, severity (as graded by National Cancer Institute Common Terminology Criteria for AEs [NCI CTCAE version 5.0]), timing, seriousness, and relationship to study drug.

The primary efficacy variable for Part 1b of the study is the proportion of patients with a decline from baseline in prostate specific antigen (PSA) blood concentrations of ≥50% at any time point during daily dosing with Compound A,

### Inclusion Criteria:

Male 18 years of age or older.
Histologically, pathologically, or cytologically confirmed prostate cancer without small cell features.
Evidence of castration-resistant prostate cancer (CRPC).
Presence of metastatic disease at study entry documented by 1 or more bone lesions on bone scan or by soft tissue disease observed by CT/MRI.
No further treatment options available known to confer clinical benefit in this disease setting from the perspective of the treating physician.
Evidence of progressive disease defined as 1 or more Prostate Cancer Working Group 3 (PCWG3) criteria.
The patient must have recovered from toxicities related to any prior treatments.
Castrate at screening.
Patients receiving bisphosphonates or other approved bone-targeting therapy must be on a stable dose for at least 4 weeks prior to the start of study drug.
Demonstrate adequate organ function.
Eastern Cooperative Oncology Group (ECOG) performance status score of 0 to 1.

### Exclusion Criteria:

Biologic anti-cancer therapy or a cytotoxic chemotherapy within 4 weeks prior to the start of study drug.
Use of hormonal agents with anti-tumor activity against prostate cancer within 4 weeks prior to the start of study drug.
Intervention with any chemotherapy, investigational agents, or other anti-cancer drugs within 14 days or 5 half-lives, whichever is longer, of the first dose of study drug
Use of radium-223 dichloride or other radioligand/radiopharmaceutical within 28 days prior to the start of study drug.
Received limited-field palliative bone radiotherapy >5 fractions and/or any radiotherapy within 2 weeks prior to the start of study drug.
Received a blood transfusion within 28 days of screening.
Known intra-cerebral disease or brain metastasis unless adequately treated and stable for the last 4 weeks before enrollment.
Spinal cord compression.
Diagnosis of another invasive malignancy within the previous 3 years other than curatively treated non-melanomatous skin cancer or superficial urothelial carcinoma.
Gastrointestinal disorder affecting absorption.
Significant cardiovascular disease.
Concurrent disease or any clinically significant abnormality.
Use of compounds known to be strong inducers and strong inhibitors of CYP3A and CYP2C8 within 14 days of the first dose of study drug.
Use of narrow therapeutic index sensitive CYP2C8 or sensitive substrates for CYP3A and CYP2B6.

PK parameters were measured from 4 subjects who were administered at 200 mg/day dose (Table 33). One patient discontinued before day 28 due to disease progression. 2 subjects received both abiraterone and enzalutamide prior treatments. 3 subjects received prior chemotherapy with taxanes.

No DLTs were observed in the 3 subjects who completed the 28 day dosing schedule at 200 mg/day and no severe adverse events were observed for the 3 subjects. Drug accumulation was observed with repeat QD dosing and steady state was reached after day 8.

TABLE 33
Measured PK parameters at 200 mg/day of Compound A

| Dose | | | t½ | Tₘₐₓ | Cₘₐₓ | Cₗₐₛₜ | AUC₀₋₂₄ |
|---|---|---|---|---|---|---|---|
| (mg/day) | Day | N | (hr) | (hr) | (ng/mL) | (ng/mL) | (ng · h/mL) |
| 200 | 1 | 4 | 22.0 | 6.5 | 3,295 | 1,808 | 53,850 |
| | 28 | 3 | 24.8 | 6.7 | 8,020 | 4,593 | 146,833 |

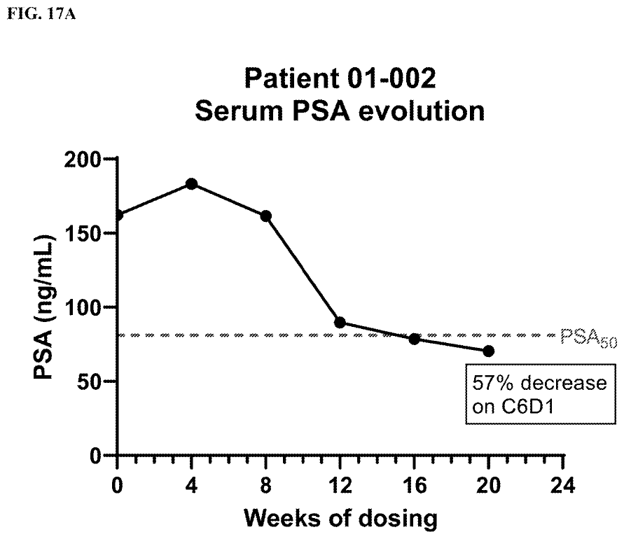

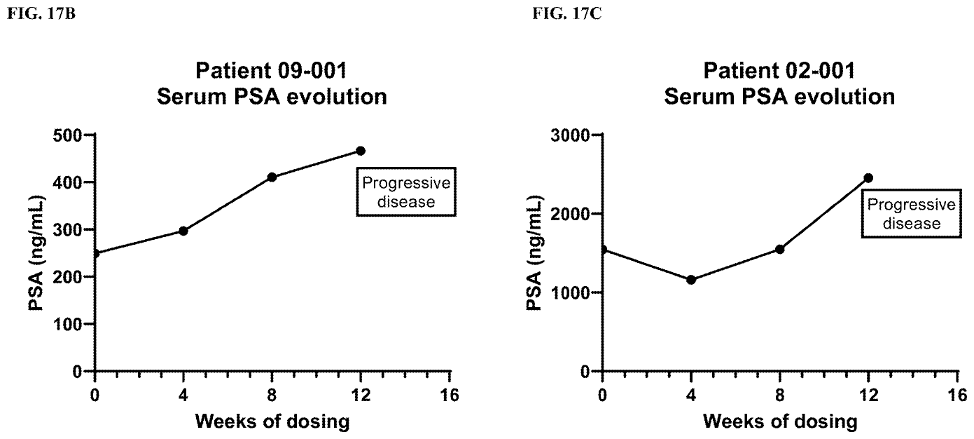

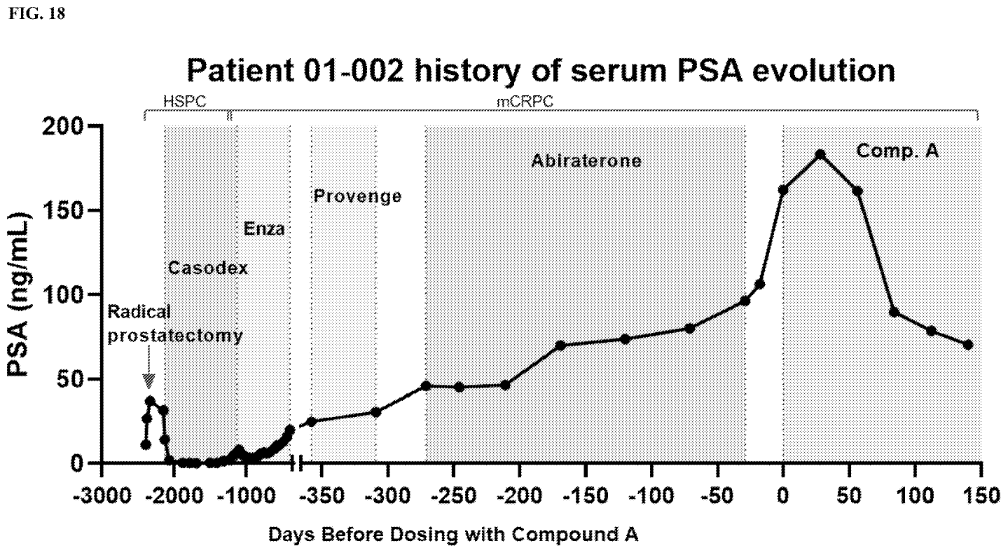

Serum PSA level decline >50% was observed in one subject after 3 cycles (12 weeks) and is ongoing (Cycle 6) (FIG. 17A). The serum PSA evolution of this subject who showed PSA level decline >50% is shown in FIG. 18 (including prior treatments). Serum PSA levels of two other subjects showed increase which is indicative of progressive disease (FIGS. 17B-17C). No dose limiting toxicity was observed among the 3 subjects. This demonstrates that 200 mg/day of Compound A was well tolerated, although the dose may not be the optimal dose to achieve antitumor efficacy. The study is still on-going with other dose levels.

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A pharmaceutical composition comprising a therapeutically effective amount of Compound A having the structure: 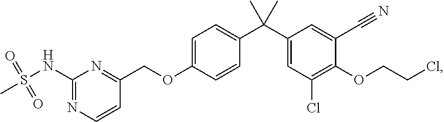 [SMILES: CC(C)(c1ccc(OCc2ccnc(NS(C)(=O)=O)n2)cc1)c1cc(Cl)c(OCCCl)c(C#N)c1]
or a pharmaceutically acceptable salt, solvate, stereoisomer or prodrug thereof, wherein the composition is a solid dispersion.

2. The pharmaceutical composition of claim 1, wherein the solid dispersion is formed by solvent evaporation, hot-melt extrusion or spray drying dispersion.

3. The pharmaceutical composition of claim 1 or 2, wherein the solid dispersion comprises one or more polymers selected from the group consisting of polyethylene glycol (PEG), polyvinyl pyrrolidone (PVP), polyethyleneoxide (PEO), poly(vinyl pyrrolidone-co-vinyl acetate) (PVP-VA), polymethacrylate, polyoxyethylene alkyl ether, polyoxyethylene-polyoxypropylene block copolymer, polyoxyethylene castor oil, polycaprolactam, polylactic acid, polyglycolic acid, poly(lactic-glycolic)acid, lipid, cellulose, pullulan, dextran, dextran acetate, dextran propionate, dextran succinate, dextran acetate propionate, dextran acetate succinate, dextran propionate succinate, dextran acetate propionate succinate, maltodextrin, hyaluronic acid, polysialic acid, chondroitin sulfate, heparin, fucoidan, pentosan polysulfate, spirulan, hydroxymethyl ethylcellulose, hydroxypropyl methylcellulose (HPMC), methyl cellulose, ethyl cellulose, hydroxyethyl cellulose, hydroxypropyl cellulose, carboxymethyl cellulose, carboxymethyl ethylcellulose (CMEC), sodium carboxymethyl cellulose, cellulose acetate succinate (CAS), methyl cellulose acetate succinate (MCAS), hydroxypropyl methylcellulose acetate succinate (HPMCAS), hydroxypropyl methylcellulose propionate succinate, hydroxypropyl methylcellulose propionate phthalate, cellulose acetate phthalate (CAP), hydroxypropyl methyl cellulose phthalate (HPMCP), hydroxypropyl methylcellulose acetate phthalate (HPMCAP), cellulose acetate trimellitate (CAT), hydroxypropyl methylcellulose acetate trimellitate (HPMCAT), hydroxypropyl methylcellulose propionate trimellitate, methyl cellulose acetate phthalate, hydroxypropyl cellulose acetate phthalate, cellulose acetate terephthalate, cellulose acetate isophthalate, cellulose acetate, cellulose butyrate, cellulose acetate butyrate, starch derivatives such as cyclodextrins (CDs), dextran polymer derivative, poly(methacrylic acid-co-methyl methacrylate) 1:1, poly(methacrylic acid-co-methyl methacrylate) 1:2, poly(methacrylic acid-co-ethyl acrylate) 1:1, and a graft copolymers comprised of polyethylene glycol, polyvinyl caprolactam and polyvinyl acetate, or any combinations thereof.

4. The pharmaceutical composition of claim 1 or 2, wherein the solid dispersion comprises one or more polymers selected from the group consisting of polyethylene glycol (PEG), polyvinyl caprolactam, polyvinyl acetate, polyvinyl pyrrolidone (PVP), poly(vinyl pyrrolidone-co-vinyl acetate) (PVP-VA), hydroxypropyl methylcellulose (HPMC), hydroxypropyl methylcellulose acetate succinate (HPMCAS), poly(methacrylic acid-co-methyl methacrylate) 1:1, and poly(methacrylic acid-co-methyl methacrylate) 1:2.

5. The pharmaceutical composition of any one of claims 1-4, wherein the composition comprises Compound A or a pharmaceutically acceptable salt, solvate, stereoisomer or prodrug thereof in an amount ranging from about 10% to about 80% by weight of the composition.

6. The pharmaceutical composition of any one of claims 1-5, wherein the composition comprises Compound A or a pharmaceutically acceptable salt, solvate, stereoisomer or prodrug thereof in an amount ranging from about 15% to about 45% by weight of the composition.

7. The pharmaceutical composition of any one of claims 1-4, wherein the weight ratio of the polymer and Compound A or a pharmaceutically acceptable salt, solvate, stereoisomer or prodrug thereof is about 80:20, about 70:30, about 65:35, about 60:40, about 55:45, about 50:50, or about 25:75.

8. The pharmaceutical composition of any one of claims 1-7, wherein the solid dispersion has a D₅₀ particle size in the range of about 10 microns to about 100 microns.

9. The pharmaceutical composition of any one of claims 1-7, wherein the solid dispersion has a D₅₀ particle size in the range of about 30 microns to about 60 microns.

10. The pharmaceutical composition of any one of claims 1-8, wherein the solid dispersion has a D₉₀ particle size in the range of about 40 microns to about 130 microns.

11. The pharmaceutical composition of any one of claims 1-8, wherein the solid dispersion has a D₉₀ particle size in the range of about 70 microns to about 100 microns.

12. The pharmaceutical composition of any one of claims 1-11, wherein the solid dispersion has a bulk density in the range of about 0.1 g/mL to about 0.6 g/mL.

13. The pharmaceutical composition of any one of claims 1-12, wherein the solid dispersion has a tap density in the range of about 0.2 g/mL to about 0.7 g/mL.

14. The pharmaceutical composition of any one of claims 1-13, wherein the solid dispersion comprises less than about 0.01 wt % dichloromethane.

15. The pharmaceutical composition ofany one of claims 1-14, wherein the solid dispersion comprises less than about 1 wt % water.

16. The pharmaceutical composition of any one of claims 1-14, wherein the solid dispersion comprises less than about 0.5 wt % water.

17. The pharmaceutical composition of any one of claims 1-16, wherein the solid dispersion exhibits a glass transition temperature (Tg) in the range of about 60° C. to about 180° C. as measured by differential scanning calorimeter.

18. The pharmaceutical composition of any one of claims 1-16, wherein the solid dispersion exhibits a glass transition temperature (Tg) in the range of about 60° C. to about 90° C. as measured by differential scanning calorimeter.

19. The pharmaceutical composition ofany one of claims 1-16, wherein the solid dispersion exhibits a glass transition temperature (Tg) in the range of about 70° C. to about 80° C. as measured by differential scanning calorimeter.

20. The pharmaceutical composition of any one of claims 1-19, wherein Compound A or a pharmaceutically acceptable salt, solvate, stereoisomer or prodrug thereof is in a crystalline form.

21. The pharmaceutical composition of any one of claims 1-19, wherein Compound A or a pharmaceutically acceptable salt, solvate, stereoisomer or prodrug thereof is in an amorphous form.

22. The pharmaceutical composition of any one of claims 1-19, wherein Compound A or a pharmaceutically acceptable salt, solvate, stereoisomer or prodrug thereof is in an amorphous form comprising less than 10% of crystalline form of Compound A or a pharmaceutically acceptable salt, solvate, stereoisomer or prodrug thereof.

23. The pharmaceutical composition of any one of claims 1-19, wherein Compound A or a pharmaceutically acceptable salt, solvate, stereoisomer or prodrug thereof is in an amorphous form comprising less than 5% of crystalline form of Compound A or a pharmaceutically acceptable salt, solvate, stereoisomer or prodrug thereof.

24. The pharmaceutical composition of any one of claims 1-23, wherein the composition exhibits an X-ray powder diffraction (XRPD) pattern substantially similar to any one of the patterns shown in FIGS. 5, 10, and 12.

25. The pharmaceutical composition of any one of claims 1-19 or 21, wherein the composition exhibits an XRPD pattern substantially similar to a pattern labeled as SDD-A, SDD-B, SDD-C, SDD-D, or SDD-E in FIG. 5 or a pattern labeled as SDD-H, SDD-I, SDD-J, SDD-N, SDD-O, SDD-O, SDD-P, SDD-Q, or SDD-R in FIG. 12.

26. The pharmaceutical composition of any one of claims 1-19 or 21, wherein the composition exhibits an XRPD pattern substantially similar to a pattern labeled as SDD-I, SDD-I, SDD-J, SDD-N, SDD-O, SDD-O, SDD-P, SDD-Q, or SDD-R in FIG. 12.

27. The pharmaceutical composition of any one of claims 1-26, wherein the composition exhibits a dissolution profile in intestinal buffer (IB) media substantially similar to any one of the profiles shown in FIGS. 8, 9, 13, and 14.

28. The pharmaceutical composition of any one of claims 1-26, wherein the composition exhibits a dissolution profile in intestinal buffer (IB) media substantially similar to the profile labeled as SDD-H, SDD-J, SDD-N, SDD-O, SDD-P, or SDD-Q in FIG. 13.

29. The pharmaceutical composition of any one of claims 1-26, wherein the composition exhibits a dissolution profile in intestinal buffer (IB) media substantially similar to any one of the profiles shown in FIG. 14.

30. The pharmaceutical composition of any one of claims 1-29, wherein the composition exhibits a modulated differential scanning calorimetry (mDSC) thermogram substantially similar to the thermogram labeled as SDD-A, SDD-B, SDD-C, SDD-D, or SDD-E in FIG. 6.

31. The pharmaceutical composition of any one of claims 1-30, wherein the composition is formulated into a capsule.

32. The pharmaceutical composition of any one of claims 1-30, wherein the composition is filled inside a tablet.

33. The pharmaceutical composition of claim 31 or 32, wherein each tablet or each capsule comprises Compound A or a pharmaceutically acceptable salt, solvate, stereoisomer or prodrug thereof in about 50 mg, about 100 mg, about 150 mg, about 200 mg, or about 250 mg.

34. The pharmaceutical composition of claim 31 or 32, wherein each tablet or each capsule comprises Compound A or a pharmaceutically acceptable salt, solvate, stereoisomer or prodrug thereof in about 5 mg and about 1000 mg, or between about 10 mg and about 500 mg, or between about 20 mg and about 250 mg, or between about 30 mg and about 300 mg, or between about 50 mg and about 200 mg.

35. The pharmaceutical composition of any one of claims 32-34, wherein the tablet has an average hardness of about 5 kP to about 35 kP.

36. The pharmaceutical composition of any one of claims 32-35, wherein the tablet has an average tensile strength of about 1 MPa to about 3 MPa.

37. The pharmaceutical composition of any one of claims 32-36, wherein the tablet has a friability of no more than 1.0% weight loss at 100 drops.

38. The pharmaceutical composition of any one of claims 32-37, wherein the tablet has an average disintegration time of less than about 300 seconds in an acidic media.

39. The pharmaceutical composition of any one of claims 32-38, wherein the tablet comprises a film coating.

40. The pharmaceutical composition of any one of claims 31-40, wherein the composition comprises a pharmaceutically acceptable excipient selected from a filler, disintegrant, glidant, or lubricant.

41. The pharmaceutical composition of any one of claims 1-40, wherein the solid dispersion comprises HPMCAS-H.

42. A method for modulating androgen receptor activity, comprising administering a pharmaceutical composition of any one of claims 1-41, to a subject in need thereof.

43. The method of claim 42, wherein the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

44. A method for treating cancer, comprising administering the pharmaceutical composition of any one of claims 1-41, to a subject in need thereof.

45. The method of claim 44, wherein the cancer is selected from prostate cancer, breast cancer, ovarian cancer, bladder cancer, pancreatic cancer, hepatocellular cancer, endometrial cancer, or salivary gland carcinoma.

46. The method of claim 44, wherein the cancer is prostate cancer.

47. The method of claim 46, wherein the prostate cancer is primary or localized prostate cancer, locally advanced prostate cancer, recurrent prostate cancer, advanced prostate cancer, metastatic prostate cancer, metastatic castration-resistant prostate cancer, and hormone-sensitive prostate cancer.

48. The method of claim 46, wherein the prostate cancer is metastatic castration-resistant prostate cancer.

49. The method of claim 46, wherein the prostate cancer expresses full-length androgen receptor or truncated androgen receptor splice variant.

50. The method of claim 46, wherein the prostate cancer is resistant to enzalutamide monotherapy.

51. The method of claim 44, wherein the cancer is breast cancer.

52. The method of claim 51, wherein the breast cancer is triple negative breast cancer.

53. An amorphous form of Compound A: 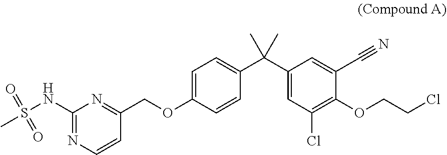 [SMILES: CC(C)(c1ccc(OCc2ccnc(NS(C)(=O)=O)n2)cc1)c1cc(Cl)c(OCCCl)c(C#N)c1]
or a pharmaceutically acceptable salt, solvate, or solvate salt thereof; wherein the amorphous form exhibits an X-ray powder diffraction (XRPD) pattern substantially similar to any one of the patterns shown in FIGS. 5, 10, and 12.

54. The amorphous form of claim 53 which exhibits an XRPD pattern substantially similar to a pattern labeled as SDD-A, SDD-B, SDD-C, SDD-D, or SDD-F in FIG. 5 or a pattern labeled as SDD-H, SDD-I, SDD-J, SDD-N, SDD-O, SDD-O, SDD-P, SDD-Q, or SDD-R in FIG. 12.

55. The amorphous form of claim 53 which exhibits an XRPD pattern substantially similar to a pattern labeled as SDD-H, SDD-I, SDD-J, SDD-N, SDD-O, SDD-O, SDD-P, SDD-Q, or SDD-R in FIG. 12.

56. The amorphous form of any one of claims 53-55, wherein the solubility is about 40 μg of Compound A/mL to about 50 μg of Compound A/mL in intestinal buffer (IB) media.

57. The amorphous form of any one of claims 53-55, wherein the solubility is about 45 μg of Compound A/mL in intestinal buffer (IB) media.

58. The amorphous form of any one of claims 53-57 which exhibits a glass transition temperature (Tg) in the range of about 60° C. to about 180° C. as measured by differential scanning calorimeter.

59. The amorphous form of any one of claims 53-57 which exhibits a glass transition temperature (Tg) in the range of about 60° C. to about 90° C. as measured by differential scanning calorimeter.

60. The amorphous form of any one of claims 53-57 which exhibits a glass transition temperature (Tg) in the range of about 60° C. to about 80° C. as measured by differential scanning calorimeter.

61. The amorphous form of any one of claims 53-60 having a purity in the range of about 80% to about 99%.

62. The amorphous form of any one of claims 53-60 having a purity of about 95% or higher.

63. The amorphous form of any one of claims 53-60 having a purity of about 99% or higher.

64. The amorphous form of any one of claims 53-61 comprising less than 10% of crystalline form of Compound A or a pharmaceutically acceptable salt, solvate, stereoisomer or prodrug thereof.

65. The amorphous form of any one of claims 53-61 comprising less than 5% of crystalline form of Compound A or a pharmaceutically acceptable salt, solvate, stereoisomer or prodrug thereof.

66. A pharmaceutical composition comprising an amorphous form of any one of claims 53-65 and a pharmaceutically acceptable excipient or carrier.

67. A method for modulating androgen receptor activity, comprising administering the amorphous form of any one of claims 53-65 or the pharmaceutical composition of claim 66, to a subject in need thereof.

68. The method of claim 67, wherein the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

69. A method for treating cancer, comprising administering the amorphous form of any one of claims 53-65 or the pharmaceutical composition of claim 66, to a subject in need thereof.

70. The method of claim 69, wherein the cancer is selected from prostate cancer, breast cancer, ovarian cancer, bladder cancer, pancreatic cancer, hepatocellular cancer, endometrial cancer, or salivary gland carcinoma.

71. The method of claim 69 or 70, wherein the cancer is prostate cancer.

72. The method of claim 71, wherein the prostate cancer is primary or localized prostate cancer, locally advanced prostate cancer, recurrent prostate cancer, advanced prostate cancer, metastatic prostate cancer, metastatic castration-resistant prostate cancer, and hormone-sensitive prostate cancer.

73. The method of claim 71, wherein the prostate cancer is metastatic castration-resistant prostate cancer.

74. The method of claim 71, wherein the prostate cancer expresses full-length androgen receptor or truncated androgen receptor splice variant.

75. The method of claim 71, wherein the prostate cancer is resistant to enzalutamide monotherapy.

76. The method of claim 69, wherein the cancer is breast cancer.

77. The method of claim 76, wherein the breast cancer is triple negative breast cancer.
